(19) United States
(12) Patent Application Publication
(10) Pub. No.: US 2023/0197278 A1
(43) Pub. Date: Jun. 22, 2023

(54) MULTI-VARIATE MODEL FOR PREDICTING CYTOKINE RELEASE SYNDROME

(71) Applicants: Genentech, Inc., South San Francisco, CA (US); Hoffmann-La Roche Inc, Little Falls, NJ (US)

(72) Inventors: Emily Piccione GRIFFIN, Belmont, CA (US); Bruce MCCALL, San Francisco, CA (US); Tina Geritz NIELSEN, Basel (CH); Anton BELOUSOV, Penzberg (DE)

(73) Assignees: Genentech, Inc., South San Francisco, CA (US); Hoffmann-La Roche Inc, Little Falls, NJ (US)

(21) Appl. No.: 17/860,939

(22) Filed: Jul. 8, 2022

Related U.S. Application Data
(60) Provisional application No. 63/221,323, filed on Jul. 13, 2021.
(60) Provisional application No. 63/263,787, filed on Nov. 9, 2021.
(60) Provisional application No. 63/341,203, filed on May 12, 2022.

(51) Int. Cl. G16H 50/30; G16H 70/60

(52) U.S. Cl. CPC G16H 50/30; G16H 70/60; G16H 20/40

(57) ABSTRACT

Techniques are provided for predicting a risk of a subject experiencing a cytokine release syndrome of at least a threshold grade subsequent to receiving a treatment. The risk may be predicted based on (for example) a set of baseline characteristics, a risk-score generation model, an on-treatment cytokine level, and/or a treatment dosage. The risk may be used to generate an output corresponding to a recommendation as to whether to monitor the subject via in-patient monitoring.

### CROSS-REFERENCE TO RELATED APPLICATIONS

This application claims the benefit of and the priority to U.S. Provisional Application No. 63/221,323, filed on Jul. 13, 2021; U.S. Provisional Application No. 63/263,787, filed on Nov. 9, 2021; and U.S. Provisional Application No. 63/341,208, filed on May 12, 2022. Each of these applications is hereby incorporated by reference in its entirety for all purposes.

### SEQUENCE LISTING

The instant application contains a Sequence Listing which has been submitted electronically in XML file format and is hereby incorporated by reference in its entirety. Said XML copy, created on Mar. 3, 2023, is named P36683-US-3_1334215_SL.xml and is 57,432 bytes in size.

### BACKGROUND

A cytokine release syndrome (and a cytokine release storm) is a potentially life threatening condition that can be caused by viral infection, autoimmune disease, and immunotherapy. Cytokine release syndrome is characterized by dramatically increased levels of cytokines and immune system dysregulation. Under normal circumstances, there is typically a balance between anti-inflammatory and pro-inflammatory cytokines. However, an excessively activated immune response can lead to significantly increased secretion of pro-inflammatory cytokines from lymphocytes (T cells, B cells, and natural killer cells) and myeloid cells (monocytes, macrophages, and dendritic cells).

The incidence of cytokine release syndrome in subjects receiving cancer immunotherapy varies widely depending on the type of immunotherapeutic agent. The onset of a cytokine release syndrome can occur within a few hours, and in the case of CAR-T cell therapy, up to several weeks after infusion of the drug. With most conventional monoclonal antibodies, the incidence of cytokine release syndrome is relatively low, whereas T cell-engaging cancer immunotherapies carry a particularly high risk of triggering a cytokine release syndrome. Therefore, a standard of care is to monitor subjects receiving immunotherapy for the symptoms of cytokine release syndrome immediately after treatment and beyond.

The risk of cytokine release syndrome is influenced by factors related to the type of therapy and the underlying disease. Many agents that can induce cytokine release syndrome display a first-dose effect, i.e., the most severe symptoms only occur after the first administered dose and do not recur after the subsequent administrations (Klinger et al. Blood 119: 6226-33 (2012)).

Despite efforts to the contrary, it is not yet possible to predict which subjects will experience a cytokine release syndrome, much less predicting any grade specifics of such an occurrence. Rather, a wide variety of clinical symptoms and the severity of cytokine-release-syndrome incidences continue to be observed, and a default is to institute consistent in-patient monitoring following administration of select therapies to facilitate quickly detecting and treating any cytokine release syndrome.

A cytokine release syndrome can cause fever, chills, fatigue, nausea, headache, muscle pain, dyspnea, tachycardia, hypotension, liver dysfunction, respiratory distress syndrome, acute vascular leak syndrome, disseminated intravascular coagulopathy, neurotoxicity, cardiac dysfunction, renal failure, and/or multiple organ failure. Mild symptoms, such as fever, nausea, fatigue, headache, and malaise, can be treated with fluids and analgesics, while continuing to monitor the subject. More severe symptoms, resulting from excessive pro-inflammatory cytokine production (i.e., a cytokine release syndrome) require rapid intervention with corticosteroids and/or anti-cytokine therapy to prevent organ damage and death. Therefore, it is important to improve the identification of risk factors for cytokine release syndrome.

### SUMMARY

In some embodiments, a method is provided that includes identifying a set of baseline characteristics of a subject who has been diagnosed with cancer, where the set of baseline characteristics pertain to one or more baseline time points that are before the initiation of the treatment, and where each of the set of baseline characteristics characterize: a stage of the cancer; a demographic attribute; a size of one or more tumors; a white blood cell count; and/or a lactate dehydrogenase level. A numeric cytokine release syndrome risk score is generated by processing the set of baseline characteristics using a risk-score generation model. A risk of the subject experiencing a cytokine release syndrome of at least a threshold grade subsequent to receiving a treatment is predicted based on the numeric cytokine release syndrome risk score. A result is determined based on the predicted risk corresponding to a recommendation as to whether to monitor the subject via in-patient monitoring subsequent to completion of the treatment. The result is output.

The method may include determining the result based on the predicted risk, where the result corresponds to a recommendation as to whether to monitor the subject via in-patient monitoring subsequent to completion of the treatment. The result may correspond to a recommendation to monitor the subject via in-patient monitoring subsequent to completion of the treatment, where the method further comprises: monitoring the subject via in-patient monitoring at a medical facility for at least 24 hours after the treatment is completed when the result indicates that the subject is at high risk for experiencing the cytokine release syndrome.

The method may include identifying an on-treatment level of a cytokine, where the on-treatment level of the cytokine indicates a level of the cytokine in an on-treatment sample collected from the subject while the treatment was being administered or within an hour of completion of the treatment; and determining an on-treatment cytokine fold change of the cytokine based on the on-treatment level of the cytokine and a baseline level of the cytokine that indicates a level of the cytokine in a baseline sample collected from the subject before initiation of the treatment; where the predicted risk is further based on the on-treatment cytokine fold change.

The method may include identifying a dosage of at least prat of the treatment, where the predicted risk is further based on the dosage.

The risk-score generation may include a regression model.

The treatment may include administering a T cell immunotherapy.

The treatment may include administering glofitamab or mosunetuzumab.

In some embodiments, a method is provided that includes identifying an on-treatment level of a cytokine, where the on-treatment level of the cytokine indicates a level of the cytokine in an on-treatment sample collected from a subject while a treatment was being administered or within an hour of completion of the treatment. An on-treatment cytokine fold change of the cytokine is determined based on the on-treatment level of the cytokine and a baseline level of the cytokine that indicates a level of the cytokine in a baseline sample collected from the subject before initiation of the treatment. A dosage of at least part of the treatment is identified. Based on the on-treatment cytokine fold change and the dosage, a risk is predicted of the subject experiencing a cytokine release syndrome of at least a threshold grade subsequent to receiving the dosage of the at least part of the treatment. A result is determined based on the predicted risk corresponding to a recommendation as to whether to monitor the subject via in-patient monitoring subsequent to completion of the treatment. The result is output.

The method may include identifying a set of baseline characteristics of the subject, where the set of baseline characteristics pertain to one or more baseline time points that are before the initiation of the treatment, and where each of the set of baseline characteristics characterize: a tumor burden; a stage of cancer; a tumor spread; a size of one or more tumors; a demographic attribute; a white blood cell count; and/or a lactate dehydrogenase level; where the predicted risk further depends on the set of baseline characteristics.

The method may include generating a cytokine release syndrome risk score by processing the set of baseline characteristics with a risk-score generation model, where the predicted risk is based on the cytokine release syndrome risk score.

The risk-score generation may include a regression model.

The one or more parameters may include a set of weights.

The risk may be determined based on a linear combination of the cytokine release syndrome risk score and the dosage.

Predicting the risk that the subject will experience the cytokine release syndrome may include performing one or more threshold comparisons.

The result may correspond to a recommendation to monitor the subject via in-patient monitoring subsequent to completion of the treatment, and the method may include monitoring the subject via in-patient monitoring at a medical facility for at least 24 hours after the treatment is completed when the result indicates that the subject is at high risk for experiencing the cytokine release syndrome.

The result may correspond to a recommendation to monitor the subject via out-patient monitoring subsequent to completion of the treatment, and the method may include monitoring the subject via out-patient monitoring when the result indicates that the subject is at low risk for experiencing the cytokine release syndrome.

The subject may have been diagnosed with cancer, and the treatment may include administering a T cell immunotherapy.

The subject may have been diagnosed with cancer, and the treatment may include administering glofitamab or mosunetuzumab.

Determining the on-treatment cytokine fold change of the cytokine based on the baseline level of the cytokine may include: calculating a log of the baseline level of the cytokine or of a processed version thereof to generate a baseline log value; calculating a log of the on-treatment level of the cytokine or a processed version thereof to generate an on-treatment log value; and subtracting the baseline log value from the on-treatment log value.

Determining the on-treatment cytokine fold change of the cytokine based on the baseline level of the cytokine may include: calculating a log of a difference between the baseline level of the cytokine and a constant to generate a baseline log value; calculating a log of a difference between the on-treatment level of the cytokine and the constant to generate an on-treatment log value; and subtracting the baseline log value from the on-treatment log value.

Identifying the on-treatment level of the cytokine may include: identifying multiple preliminary on-treatment levels of the cytokine that indicate levels of the cytokine in multiple on-treatment samples collected from the subject while the treatment was being administered or within a day of completion of the treatment, where each of the multiple on-treatment samples was collected at a different time; and defining the on-treatment level of the cytokine to be a maximum of the multiple preliminary on-treatment levels of the cytokine.

The treatment may include administering an active ingredient; and the treatment may have been preceded by administering a pre-treatment with another agent.

The on-treatment level may have been identified using a sample collected after the administration of the active ingredient.

The cytokine may include Tumor Necrosis Factor alpha, Interleukin 6, Interleukin 8, Interleukin 10, or Macrophage Inflammatory Protein 1 beta.

The on-treatment level of the cytokine may have been determined by: collecting a blood sample from the subject while the treatment was being administered; and processing the blood sample using capture and detection antibodies for the cytokine.

In some embodiments, a method is provided that includes determining a baseline level of a cytokine that indicates a level of a cytokine in a baseline sample collected from a subject before initiation of a treatment; determining an on-treatment level of the cytokine, where the on-treatment level of the cytokine indicates a level of the cytokine in an on-treatment sample collected from the subject while the treatment was being administered or within an hour of completion of the treatment; and identifying a dosage of at least part of the treatment. Further, the baseline level of the cytokine and the on-treatment level of the cytokine is input to a computing system. A result is received that corresponds to a recommendation to monitor the subject via in-patient monitoring subsequent to completion of the treatment; and the subject is monitored via in-patient monitoring subsequent to completion of the treatment.

The subject may be monitored via in-person monitoring for at least 4 hours subsequent to the completion of the treatment.

The result may be generated by the computing system by: determining an on-treatment cytokine fold change of the cytokine based on a baseline level of the cytokine and the on-treatment level of cytokine; and predicting, based on the on-treatment cytokine fold change and the dosage, a risk of the subject experiencing a cytokine release syndrome of at least a threshold grade subsequent to receiving the dosage of the at least part of the treatment.

In some embodiments, a method is provided that includes determining a baseline level of a cytokine that indicates a level of a cytokine in a baseline sample collected from a subject before initiation of a treatment; determining an on-treatment level of the cytokine, where the on-treatment level of the cytokine indicates a level of the cytokine in an on-treatment sample collected from the subject while the treatment was being administered or within an hour of completion of the treatment; and identifying a dosage of at least part of the treatment. Further, the baseline level of the cytokine and the on-treatment level of the cytokine is input to a computing system; a result is received that corresponds to a recommendation to monitor the subject via out-patient monitoring subsequent to completion of the treatment; and the subject is monitored via out-patient monitoring subsequent to completion of the treatment.

The subject may be monitored via in-person monitoring for at least 4 hours subsequent to the completion of the treatment.

The result may be generated by the computing system by: determining an on-treatment cytokine fold change of the cytokine based on a baseline level of the cytokine and the on-treatment level of cytokine; and predicting, based on the on-treatment cytokine fold change and the dosage, a risk of the subject experiencing a cytokine release syndrome of at least a threshold grade subsequent to receiving the dosage of the at least part of the treatment.

In some embodiments, use of a computational prediction is provided to determine whether to monitor a subject via in-patient monitoring for a cytokine release syndrome after administration of a treatment, where the computational prediction is provided by a computing device implementing a risk-score generation model that: determines an on-treatment cytokine fold change of a cytokine based on: a baseline level of the cytokine that indicates that a level of the cytokine in a baseline sample collected from the subject before initiation of the treatment; and an on-treatment level of the cytokine that indicates a level of the cytokine in an on-treatment sample collected from the subject while the treatment was being administered or within an hour of completion of the treatment; and predicts a risk of the subject experiencing a cytokine release syndrome of at least a threshold grade after administration of the treatment based on the on-treatment cytokine fold change.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

With respect to any method, use, system, or computer-program product disclosed herein, the treatment may comprise administering a therapy that comprises an antibody or a small molecule.

The administered therapy may comprise an antibody.

The antibody may specifically bind CD20, CD52, CD30, CD40, or PD-1.

The antibody may be rituximab, obinutuzumab, alemtuzumab, brentuximab, dacetuzumab, or nivolumab.

The antibody may be a multispecific antibody that engages T-cells when bound to at least one of its antigens.

The multispecific antibody may specifically bind at least CD3.

The multispecific antibody may further specifically bind at least CD20.

The multispecific antibody may be a bispecific antibody.

The bispecific antibody may specifically bind CD3 and/or CD20.

The bispecific antibody may be mosunetuzumab or glofitamab.

The therapy may comprise a small molecule, such as is oxaliplatin or lenalidomide.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows an exemplary network for stratifying subjects for differential monitoring or treatment by predicting a risk of one or more individual subjects experiencing a cytokine risk syndrome event according to some embodiments.

FIG. 2A illustrates a flowchart of a process for predicting a risk that a subject will experience a cytokine release syndrome.

FIG. 2B shows a process for using a predicted risk to determine whether to recommend in- or out-patient monitoring for cytokine release syndromes in a subject.

FIG. 3 represents the dosage timing in various cohorts who received a treatment comprising obinutuzumab and glofitamab.

FIG. 4 shows a representation of exemplary data used to train and validate a feature selection model (to identify a reduced feature set and thresholds to convert any non-binary baseline characteristic into a binary variable), a risk-score generation model (to convert binary values from the reduced feature set into a risk score), and a decision-tree model (to convert the risk score and cytokine fold changes to a prediction as to whether a grade 2+ cytokine release syndrome will occur).

FIG. 5 shows the timing of cytokine release syndromes for each exemplary analysis cohort.

FIG. 6 shows the percentage of subjects in exemplary training and validation data sets within each cohort that experienced a cytokine release syndrome event during the first week of Cycle 1.

FIG. 7 shows an exemplary workflow used to identify the extent to which of various baseline characteristics (or “risk factors”) contributed to a prediction of occurrence of cytokine release syndrome and how parameters in a model were learned.

FIG. 8 is a forest plot that shows the degree to which each of multiple baseline characteristics was predictive of occurrence of cytokine release syndrome (of Grade 2+ after the first glofitamab dosage) in an exemplary data set.

FIG. 9A illustrates how a multivariate logistic regression models can be used to predict the cytokine release risk.

FIG. 9B illustrates how the cytokine release risk can be is calculated and used, together with the dosage in the predictive model.

FIG. 10 shows exemplary negative predicted values (NPV) relative to the predicted negative cases corresponding to risk scores from two versions of the risk-score generation model.

FIG. 11 shows exemplary negative predictive value versus predicted negative cases for a validation data set of the 2.5/10/30 mg step-up dosage cohort.

FIG. 12A shows exemplary probabilities of a cytokine release syndrome (of Grade 2 or above after the first glofitamab dose) occurring as a function of the cytokine release syndrome risk score (CRSRS) for each of three exemplary thresholds that differentiate whether it is a predicted that the event would have or would not have occur.

FIG. 12B shows statistics pertaining to predictions generated by using a trained decision tree model-to process a validation data set.

FIG. 13 shows the distribution of exemplary baseline cytokine release syndrome risk score (CRSRS) values corresponding to clinical study NP30179.

FIGS. 14A and 14B show the exemplary fold changes of IL-6 and TNF-α (respectively) during a first glofitamab treatment cycle.

FIG. 15 contrasts the cytokine fold changes of IL-6 for exemplary subjects who did not experience a cytokine release syndrome (left plot) and for exemplary subjects who did experience a cytokine release syndrome (right plot).

FIGS. 16A-16B shows box plots indicating how exemplary on-treatment cytokine fold changes depended on the existence or grade of the first cytokine release syndrome.

FIGS. 17A-17B show how exemplary on-treatment levels of each of two cytokines (IL-6, TNF-α) change across the first cycle of glofitamab treatment.

FIGS. 18A-18B show the maximum log 2 fold-changes across exemplary subjects for IL-6 and TNF-α, respectively.

FIGS. 19A-19B were generated to show exemplary time courses of cytokine fold changes across various treatment-related periods, while stratifying subjects according to time of onset of cytokine release syndrome relative to treatment initiation.

FIGS. 20A-20B show exemplary time courses of cytokine fold changes and boxplots that compare cytokine for changes across instances differentiated based on whether any type of cytokine release syndrome occurred or based on whether a cytokine release syndrome of at least Grade 2 occurred.

FIGS. 21A-21B compare exemplary fold-changes of cytokines to cytokine release syndrome risk scores for different dosage groups.

FIG. 22 shows results from a landmark analysis of how the probability of a grade 2 or higher cytokine release syndrome occurring (adjusted for the first glofitamab dosage) relates to a normalized version the cytokine release syndrome risk score.

FIG. 23 exemplifies how the grade of any observed cytokine release syndrome relates to both the cytokine release syndrome risk score and the cytokine fold change of TNF-α.

FIG. 24 shows the negative predictive value and low-risk detection rate for a set of cut-off values in a step-up (model-validation) dosage cohort for the full 8-parameter score and the reduced, 5-parameter score CRSRS.5p.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

Techniques disclosed herein relate to using a multivariate analysis to predict whether a subject will experience a cytokine release syndrome (e.g., of at least a predefined severity) based on baseline or on-treatment data points. The prediction may include predicting whether it is determined that a subject is of low risk of experiencing cytokine release syndrome (e.g., of at least a threshold grade) and/or is a candidate for out-patient monitoring for cytokine release syndrome. This predication may be made after optimizing a negative predictive value of a model used to generate an output that predicts occurrence of cytokine release syndrome (e.g., of at least a threshold grade). Alternatively or additionally, the prediction may include predicting whether it is determined that a subject is at risk of experiencing cytokine release syndrome (e.g., of at least a threshold grade) and/or is a candidate for in-patient monitoring for cytokine release syndrome. This predication may be made after optimizing a positive predictive value of a model used to generate an output that predicts occurrence of cytokine release syndrome (e.g., of at least a threshold grade).

The baseline data point(s) may be associated with one or more time points before the treatment has been initiated and/or between a pre-treatment and a first non-pre-treatment dosage of another active ingredient. For example, a baseline data point may have been generated by processing a sample collected before administration of a first dosage of the active ingredient, and/or a baseline data point may have been retrieved from a medical record associated with an assessment performed at a time before a first non-pre-treatment dosage of the active ingredient. The on-treatment data point(s) may be associated with one or more time points between initiation of the treatment and termination of the treatment (potentially plus a buffer). For example, an on-treatment data point may be defined to equal a maximum concentration level of a particular cytokine across all measurements made after a first (e.g., non-priming) dosage of the active ingredient and a termination of the treatment (potentially plus a buffer).

The multivariate analysis and risk predictor can include a change of a level of a particular cytokine at an on-treatment time point (or a processed version thereof) from a level of the particular cytokine at a baseline time point (or a processed version thereof. For example, a processed version of a cytokine level may be defined to include a log (e.g., log base 2) of a sum of the cytokine level and a non-zero, positive constant (e.g., 1).

The multivariate analysis can include generating a cytokine release syndrome risk score based on one or more baseline characteristics of the subject (associated with one or more baseline time points). The baseline characteristics may include one or more metrics characterizing tumor burden; one or more metrics characterizing tumor spread; one or more metrics characterizing a presence or degree of malignant cells being within a defined bodily component (e.g., bone marrow or peripheral blood), one or more demographic attributes (e.g., age), and/or one or more metrics characterizing of an incidence or severity of a comorbidity. The cytokine release syndrome risk score may further or alternatively be generated based on a dosage of the active ingredient in the treatment.

Generating the cytokine release syndrome risk score can include using a multivariate regression model (e.g., a linear regression model or a logistic regression model) to transform the one or more baseline characteristics (or multiple baseline characteristics) into a model output. The model output can include a scaled or unscaled representation of a risk of experiencing a cytokine release syndrome. The model output may be normalized. For example, the model output may be a number between 0 and 1, where a value of 1 represents a highest predicted risk of a cytokine release syndrome occurring, and a value of 0 represents a lowest predicted risk of a cytokine release syndrome occurring.

The multivariate model can incorporate an output from other machine learning modules (e.g., a random forest model). The multivariate model can include a set of parameters, where a value for each of the parameters was learned by training the multivariate model and multivariate machine learning model using a training data set. The set of parameters (e.g., a set of model weights) may include, for each of the one or more baseline characteristics, one or more associated parameters, where the one or more parameters may identify a degree to which the model output depends on the baseline characteristic and/or a significance value representing a degree to which the baseline characteristic is predictive of the model output.

The final cytokine release syndrome risk predictor can be generated based on the terms that identify or were derived from baseline characteristics (e.g., the parameters of the cytokine release syndrome risk score) and based on another term that identifies or derived from a dosage (e.g., of the treatment or of the active ingredient) or drug exposure. For example, the combined cytokine release syndrome risk score can be defined to be a linear combination, sum, or weighted sum of the cytokine release syndrome risk score and of the dosage/exposure.

With the help of the predictive model that combines risk factors and dose/exposure information, for every subject with a given (e.g. accessed at baseline) value of the cytokine release syndrome risk score, the dose or exposure can be adjusted in such a way as to limit the expected risk of cytokine release syndrome.

A cytokine release syndrome predictive model may be extended using one or more cytokine fold changes. For example, the cytokine release syndrome risk may be predicted based on the cytokine release syndrome risk score and the cytokine fold change(s). As another example, the cytokine release syndrome risk may be predicted based on the cytokine release syndrome risk score, the dosage/exposure, and the cytokine fold change(s). In another example, the cytokine release syndrome risk may be predicted based on the dosage and the cytokine fold change(s). As yet another example, for a particular subject the cytokine release syndrome risk may be optimized (restricted) by selecting the maximal dose/exposure at which the cytokine release syndrome risk predicted based on the score, dosage, and the cytokine fold change(s) does not exceed a certain pre-defined value. The risk may be a numeric risk (e.g., representing a probability), a categorical risk (e.g., high, medium, or low), or a binary risk (e.g., at risk or not). A categorical or binary risk may be generated based on one or more threshold comparisons. For example, it may be determined that a subject is at high risk of experiencing a cytokine release syndrome risk score exceeds a risk-score threshold and/or if a cytokine fold change exceeds a cytokine threshold, and that the subject is otherwise at a low risk of experiencing a cytokine release syndrome.

A result that corresponds to the prediction may be output (e.g., presented or transmitted). The result may include the predicted cytokine release syndrome risk. The result may include a recommended action, default action, or action to be implemented.

The cytokine release syndrome risk can be used to identify an action for recommended monitoring the subject upon termination of the treatment or to identify a recommendation as to such an approach. For example, it may be recommended that a given subject have in-patient monitoring (e.g., such that the subject is admitted to a medical facility) if an in-patient monitoring condition is satisfied. The in-patient monitoring condition may be configured to be satisfied if (for example) the risk is defined to be high; the risk is defined to be a category other than low; the risk is above a predefined (e.g., numeric or categorical) threshold. In some instances, in-patient monitoring for the subject is provided if the risk is high, the risk is category other than low, or the risk is above a predefined risk threshold.

It may alternatively or additionally be recommended that a given subject be released and/or have out-patient monitoring (e.g., such that the subject is admitted to a medical facility) if an in-patient monitoring condition is not satisfied. In some instances, subject release and/or out-patient monitoring for the subject is provided if the risk is low, the risk is category other than high, or the risk does not exceed the predefined risk threshold.

Determining cytokine release syndrome risks (e.g., with few false negatives) has an advantage of facilitating efficiently allocating resources for in-patient monitoring. Cautiously monitoring all subjects with in-patient monitoring is expensive, consumes substantial physical resources, and is time-intensive. Meanwhile, being over-inclusive in which subjects are to have out-patient monitoring can result in select subjects not being able to receive prompt treatment of a cytokine release syndrome. Thus, techniques disclosed herein can facilitate efficient resource usage that prioritizes providing resource-intensive monitoring for subjects with a relatively high risk of experiencing a cytokine release syndrome, while reserving less resource-intensive monitoring for subjects unlikely to need prompt intervention (e.g., response to a cytokine release syndrome).

### II. Definitions

The term “cytokine”, as used herein, refers to a signaling molecule that is transiently produced, after cellular activation, to help mediate and regulate immunity, inflammation and hematopoiesis. A cytokine can be any of a large group of proteins, peptides and glycoproteins that is secreted by specific cells of the immune system These molecules act as regulators that modulate the functions of individual cells. Cytokines can act locally, as autocrine, paracrine or endocrine response modifiers, and their actions are exerted via specific cell-surface receptors of their target cells. As used herein, “autocrine” or “autocrine action”, means that a cytokine exerts its action by binding to a receptor on the membrane of the same cells that secreted it. “Paracrine” or “paracrine action” means that a cytokine binds or a receptor on a target cell in close proximity to a cell that produced the cytokine. “Endocrine” or “endocrine action” means that a cytokine travels through circulation and acts on target cells in parts throughout the body. Elevated levels of cytokines, for example, one or more cytokines selected from the group consisting of IL-1β, IL-2, IL-6, IL-8, MIP1b, MCP1, IL-10, IFN-γ, TGF-β, and TNF-α, are often associated with cytokine release syndrome.

The term “cytokine release syndrome” or “CRS” as used herein, refers to an acute systemic inflammatory syndrome characterized by fever and multiple organ dysfunction that is associated with immunotherapy, for example, T-cell immunotherapies, therapeutic antibodies, chimeric antigen receptor (CAR)-T cell therapy, and stem cell transplantation. CRS is a potentially life-threatening cytokine-associated toxicity that can occur as a result of cancer immunotherapy. CRS results from high-level immune activation when large numbers of lymphocytes and/or myeloid cells release inflammatory cytokines upon activation, characterized by elevated circulating cytokine levels, acute systemic inflammatory symptoms. The severity of CRS and the timing of onset of symptoms vary depending on the degree of immune cell activation, the type of administered therapy, and tumor burden. Symptoms of CRS can include neurologic toxicity, cardiac dysfunction, disseminated intravascular coagulation, adult respiratory distress syndrome, renal failure, and hepatic failure. Symptoms can include fever (with or without rigors (“shaking chills”-temperature rises with shivering and chills), fatigue, malaise, myalgias (muscle pain), vomiting, headache, nausea, anorexia, arthalgias (joint pain), diarrhea, rash, hypoxemia (low blood oxygen), tachypnea (rapid breathing), hypotension, widened pulse pressure (the difference between systolic and diastolic blood pressures), potentially diminished cardiac output (late), increased cardiac output (early), azotemia (high concentration of nitrogen substances in the blood), hypofibrinogenemia (blood coagulation disorder; with or without bleeding), elevated D-dimer (correlating to blood clots), hyperbilirubinemia (excess blood bilirubin from red blood cells breaking down), transaminitis (elevated transminases in the blood, correlating to liver disease and hepatitis), confusion, delirium, mental status changes, hallucinations, tremor, seizures, altered gait, word finding difficulty, frank aphasia (language impairment affecting speech and/or comprehension, and writing), or dymetria (inability to adjust accurately movements, without visual assistance). CRS is characterized by inflammation beyond that which could be attributed to a normal response to a pathogen (if a pathogen is present), or any cytokine-driven organ dysfunction (if no pathogen is present).

The term “in-patient monitoring”, as used herein, refers to monitoring performed by one or more medical care providers (e.g., one or more physicians and/or one or more nurses) provided at a medical facility for a subject also concurrently at the medical facility. Thus, the subject and at least one medical care provider can be physically at the same medical facility at the same time. The medical facility may include (for example) a hospital, medical clinic, physician's office, or medication infusion center. The subject may be admitted to the medical facility during the in-patient monitoring. A duration of in-patient monitoring may be at least (for example): 2 hours, 4 hours, 8 hours, 12 hours, 24 hours, 36 hours, 48 hours, 72 hours, or 96 hours. A duration of in-patient monitoring may less than (for example): 2 weeks, 1 week, 5 days, 4 days, 3 days, or 2 days. For example, a subject may receive in-patient monitoring for between 2 and 4 days after treatment administration concluded.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

The terms “full-length antibody,” “intact antibody,” and “whole antibody” are used herein interchangeably to refer to an antibody having a structure substantially similar to a native antibody structure or having heavy chains that contain an Fc region as defined herein.

By “binding domain” is meant a part of a compound or a molecule that specifically binds to a target epitope, antigen, ligand, or receptor. Binding domains include but are not limited to antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

In certain aspects, an antibody is a multispecific antibody, e.g., a bispecific antibody. “Multispecific antibodies” are monoclonal antibodies that have binding specificities for at least two different sites, i.e., different epitopes on different antigens or different epitopes on the same antigen. A multispecific antibody can also have three or more binding specificities. Multispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO2011/034605); using the common light chain technology for circumventing the light chain mis-pairing problem (see, e.g., WO98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig can also be used in the disclosed methods (see, e.g., WO2001/77342 and WO2008/024715). Other examples of multispecific antibodies with three or more antigen binding sites can be found in WO2010/115589, WO2010/112193, WO2010/136172, WO2010/145792, and WO2013/026831. The bispecific antibody or antigen binding fragment thereof also includes a “Dual Acting FAb” or “DAF” (see, e.g., US 2008/0069820 and WO2015/095539).

Multi-specific antibodies may also be provided in an asymmetric form with a domain crossover in one or more binding arms of the same antigen specificity, i.e. by exchanging the VH/VL domains (see e.g., WO2009/080252 and WO2015/150447), the CH1/CL domains (see e.g., WO2009/080253) or the complete Fab arms (see e.g., WO2009/080251, WO2016/016299, also see Schaefer et al, Proc. Natl. Acad. Sci. USA, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO2016/172485.

Various further molecular formats for multispecific antibodies are known in the art (see e.g., Spiess et al., Mol Immunol 67 (2015) 95-106).

A particular type of multispecific antibody can recruit T cells, T-cell engaging antibodies. “T-cell bispecific antibodies” are a type of multispecific antibody, a bispecific antibody, that is engineered to bind two different antigens, where one targets tumor cells and the other one targets effector cells, usually T-lymphocytes. When the T-cell bispecific antibodies bind to a T cell and a tumor cell, the tumor cell and the T cell are brought into proximity, T cell is activated and mediates tumor cell destruction.

Examples of bispecific antibody formats include “BiTE” (bispecific T cell engager) molecules wherein two scFv molecules are fused by a flexible linker (see, e.g., WO2004/106381, WO2005/061547, WO2007/042261, and WO2008/119567, Nagorsen and Bauerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats are described in WO2013/026833, WO2013/026839, WO2016/020309; Bacac et al., Oncoimmunology 5(8) (2016) e1203498.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (Kd) of ≤1 μM, ≤100 nM, ≤10 nM, ≤1 nM, ≤0.1 nM, ≤0.01 nM, or ≤0.001 nM (e.g., 10-8 M or less, e.g., from 10-8 M to 10-13 M, e.g., from 10-9 M to 10-13 M). In certain embodiments, an anti-CD3 antibody binds to an epitope of CD3 that is conserved among CD3 from different species.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724; SEQ ID NO:45), which is 207 amino acids in length, and human CD3γ protein (NCBI RefSeq No. NP_000064; SEQ ID NO:46), which is 182 amino acids in length.

The terms “anti-CD20 antibody” and “an antibody that binds to CD20” refer herein to an antibody that is capable of binding CD20 with sufficient affinity such that the antibody is useful as therapeutic agent in targeting CD20. In one embodiment, the extent of binding of an anti-CD20 antibody to an unrelated, non-CD20 protein is less than about 10% of the binding of the antibody to CD20 as measured, e.g., by a radioimmunoassay (RIA). In certain embodiments, an antibody that binds to CD20 has a dissociation constant (Kd) of ≤1 μM, ≤100 nM, ≤10 nM, ≤1 nM, ≤0.1 nM, ≤0.01 nM, or ≤0.001 nM (e.g., 10-8 M or less, e.g., from 10-8 M to 10-13 M, e.g., from 10-9 M to 10-13 M). In certain embodiments, an anti-CD20 antibody binds to an epitope of CD20 that is conserved among CD20 from different species.

The term “cluster of differentiation 20” or “CD20,” as used herein, refers to any native CD20 from any vertebrate source, including mammals such as primates (e.g., humans) and rodents (e.g., mice and rats), unless otherwise indicated. The term encompasses “full-length,” unprocessed CD20, as well as any form of CD20 that results from processing in the cell. The term also encompasses naturally occurring variants of CD20, including, for example, splice variants or allelic variants. CD20 includes, for example, human CD20 protein (see, e.g., NCBI RefSeq Nos. NP 068769.2 (SEQ ID NO:47) and NP_690605.1 (SEQ ID NO:48)), which is 297 amino acids in length and may be generated, for example, from variant mRNA transcripts that lack a portion of the 5′ UTR (see, e.g., NCBI RefSeq No. NM_021950.3 (SEQ ID NO:49)) or longer variant mRNA transcripts (see, e.g., NCBI RefSeq No. NM_152866.2 (SEQ ID NO:50)).

The terms “anti-CD20/anti-CD3 bispecific antibody,” “bispecific anti-CD20/anti-CD3 antibody,” and “antibody that binds to CD20 and CD3,” or variants thereof, refer to a multispecific antibody (e.g., a bispecific antibody) that is capable of binding to CD20 and CD3 with sufficient affinity such that the antibody is useful as a diagnostic and/or therapeutic agent in targeting CD20 and/or CD3. In one embodiment, the extent of binding of a bispecific antibody that binds to CD20 and CD3 to an unrelated, non-CD3 protein and/or non-CD20 protein is less than about 10% of the binding of the antibody to CD3 and/or CD20 as measured, e.g., by a radioimmunoassay (RIA). In certain embodiments, a bispecific antibody that binds to CD20 and CD3 has a dissociation constant (Kd) of ≤1 μM, ≤100 nM, ≤10 nM, ≤1 nM, ≤0.1 nM, ≤0.01 nM, or ≤0.001 nM (e.g., 10-8 M or less, e.g., from 10-8 M to 10-13 M, e.g., from 10-9 M to 10-13 M). In certain embodiments, a bispecific antibody that binds to CD20 and CD3 binds to an epitope of CD3 that is conserved among CD3 from different species and/or an epitope of CD20 that is conserved among CD20 from different species.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (KD) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a KD for the target of 10-4 M or lower, alternatively 10-5 M or lower, alternatively 10-6 M or lower, alternatively 10-7 M or lower, alternatively 10-8 M or lower, alternatively 10-9 M or lower, alternatively 10-10 M or lower, alternatively 10-11 M or lower, alternatively 10-12 M or lower or a KD in the range of 10-4 M to 10-6 M or 10-6 M to 10-10 M or 10-7 M to 10-9 M. As will be appreciated by the skilled artisan, affinity and KD values are inversely related. A high affinity for an antigen is measured by a low KD value. In one embodiment, the term “specific binding” refers to binding where a molecule binds to a particular polypeptide or epitope on a particular polypeptide without substantially binding to any other polypeptide or polypeptide epitope.

The disclosed methods can be used when therapeutic bispecific antibodies that bind to CD20 and CD3 (i.e., anti-CD20/anti-CD3 antibodies) are used to treat CD20-positive cell proliferative disorders, e.g., a B cell proliferative disorder (e.g., non-Hodgkin's lymphoma (NHL) (e.g., a diffuse-large B cell lymphoma (DLBCL) (e.g., relapsed and/or refractory DLBCL or a Richter's transformation), a follicular lymphoma (FL) (e.g., a relapsed and/or refractory FL or a transformed FL), a mantle cell lymphoma (MCL), a high-grade B cell lymphoma, or a primary mediastinal (thymic) large B cell lymphoma (PMLBCL)) or a chronic lymphoid leukemia (CLL).

In some instances, the anti-CD20/anti-CD3 bispecific antibody is mosunetuzumab, having the International Nonproprietary Names for Pharmaceutical Substances (INN) List 117 (WHO Drug Information, Vol. 31, No. 2, 2017, p. 303), or CAS Registry No. 1905409-39-3, and having (1) an anti-CD20 arm comprising the heavy chain and light chain sequences of SEQ ID NOs:17 and 18, respectively; and (2) an anti-CD3 arm comprising the heavy chain and light chain sequences of SEQ ID NOs:19 and 20, respectively. In some instances, the anti-CD20/anti-CD3 bispecific antibody comprises (1) an anti-CD20 arm comprising a first binding domain comprising a heavy chain comprising an amino acid sequence of SEQ ID NO: 17 and a light chain comprising an amino acid sequence of SEQ ID NO: 18 and (2) an anti-CD3 arm comprising a second binding domain comprising a heavy chain comprising an amino acid sequence of SEQ ID NO:19 and a light chain comprising an amino acid sequence of SEQ ID NO:20. The various elements (HVRs, VHs, VLs, HCs, and LCs for mosunetuzumab are shown in Table 1.

The anti-CD20/anti-CD3 bispecific antibody may be produced using recombinant methods and compositions, for example, as described in U.S. Pat. No. 4,816,567.

TABLE 1
Amino acid sequences for mosunetuzumab

| | | |
|---|---|---|
| Description | Sequence | SEQ ID NO: |

CD20 arm

| | | |
|---|---|---|
| CD20 HVR-H1 | GYTFTSYNMH | 1 |
| CD20 HVR-H2 | AIYPGNGDTS YNQKFKG | 2 |
| CD20 HVR-H3 | WYYSNSYWY FDV | 3 |
| CD20 HVR-L1 | RASSSVSYMH | 4 |
| CD20 HVR-L2 | APSNLAS | 5 |
| CD20 HVR-L3 | QQWSFNPPT | 6 |
| CD20 VH | EVQLVESGGG LVQPGGSLRL SCAASGYTET | 7 |
| | SYNMHWVRQA PGKGLEWVGA IYPGNGDTSY | |
| | NQKFKGRFTI SVDKSKNTLY LQMNSLRAED | |
| | TAVYYCARVV YYSNSYWYFD VWGQGTLVTV | |
| | SS | |
| CD20 VL | DIQMTQSPSS LSASVGDRVT ITCRASSSVS | 8 |
| | YMHWYQQKPG KAPKPLIYAP SNLASGVPSR | |
| | FSGSGSGTDF TLTISSLQPE DFATYYCQQW | |
| | SFNPPTFGQG TKVEIK | |
| CD20 heavy chain | EVQLVESGGG LVQPGGSLRL SCAASGYTFT | 17 |
| | SYNMHWVRQA PGKGLEWVGA IYPGNGDTSY | |
| | NQKFKGRFTI SVDKSKNTLY LQMNSLRAED | |
| | TAVYYCARVV YYSNSYWYFD VWGQGTLVTV | |
| | SSASTKGPSV FPLAPSSKST SGGTAALGCL | |
| | VKDYFPEPVT VSWNSGALTS GVHTFPAVLQ | |
| | SSGLYSLSSV VTVPSSSLGT QTYICNVNHK | |
| | PSNTKVDKKV EPKSCDKTHT CPPCPAPELL | |
| | GGPSVFLFPP KPKDTLMISR TPEVTCVVVD | |
| | VSHEDPEVKF NWYVDGVEVH NAKTKPREEQ | |
| | YGSTYRVVSV LTVLHQDWLN GKEYKCKVSN | |
| | KALPAPIEKT ISKAKGQPRE PQVYTLPPSR | |
| | EEMTKNQVSL WCLVKGFYPS DIAVEWESNG | |
| | QPENNYKTTP PVLDSDGSFF LYSKLTVDKS | |
| | RWQQGNVFSC SVMHEALHNH YTQKSLSLSP GK | |
| CD20 light chain | DIQMTQSPSS LSASVGDRVT ITCRASSSVS | 18 |
| | YMHWYQQKPG KAPKPLIYAP SNLASGVPSR | |
| | FSGSGSGTDF TLTISSLQPE DFATYYCQQW | |
| | SFNPPTFGQG TKVEIKRTVA APSVFIFPPS | |
| | DEQLKSGTAS VVCLLNNFYP REAKVQWKVD | |
| | NALQSGNSQE SVTEQDSKDS TYSLSSTLTL | |
| | SKADYEKHKV YACEVTHQGL SSPVTKSFNR GEC | |

CD3 arm

| | | |
|---|---|---|
| CD3 HVR-H1 | NYYIH | 9 |
| CD3 HVR-H2 | WIYPGDGNTK YNEKFKG | 10 |
| CD3 HVR-H3 | DSYSNYYFDY | 11 |
| CD3 HVR-L1 | KSSQSLLNSR TRKNYLA | 12 |
| CD3 HVR-L2 | WASTRES | 13 |
| CD3 HVR-L3 | TQSFILRT | 14 |
| CD3 VH | EVQLVQSGAE VKKPGASVKV SCKASGYTFT | 15 |
| | NYYIHWVRQA PGQGLEWIGW IYPGDGNTKY | |
| | NEKFKGRATL TADTSTSTAY LELSSLRSED | |
| | TAVYYCARDS YSNYYFDYWG QGTLVTVSS | |
| CD3 VL | DIVMTQSPDS LAVSLGERAT INCKSSQSLL | 16 |
| | NSRTRKNYLA WYQQKPGQPP KLLIYWASTR | |
| | ESGVPDRFSG SGSGTDFTLT ISSLQAEDVA | |
| | VYYCTQSFIL RTFGQGTKVE IK | |
| CD3 heavy chain | EVQLVQSGAE VKKPGASVKV SCKASGYTFT | 19 |
| | NYYIHWVRQA PGQGLEWIGW IYPGDGNTKY | |
| | NEKFKGRATL TADTSTSTAY LELSSLRSED | |
| | TAVYYCARDS YSNYYFDYWG QGTLVTVSSA | |
| | STKGPSVFPL APSSKSTSGG TAALGCLVKD | |
| | YFPEPVTVSW NSGALTSGVH TFPAVLQSSG | |
| | LYSLSSVVTV PSSSLGTQTY ICNVNHKPSN | |
| | TKVDKKVEPK SCDKTHTCPP CPAPELLGGP | |
| | SVFLFPPKPK DTLMISRTPE VTCVVVDVSH | |
| | EDPEVKFNWY VDGVEVHNAK TKPREEQYGS | |
| | TYRVVSVLTV LHQDWLNGKE YKCKVSNKAL | |
| | PAPIEKTISK AKGQPREPQV YTLPPSREEM | |
| | TKNQVSLSCA VKGFYPSDIA VEWESNGQPE | |
| | NNYKTTPPVL DSDGSFFLVS KLTVDKSRWQ | |
| | QGNVFSCSVM HEALHNHYTQ KSLSLSPGK | |
| CD3 light chain | DIVMTQSPDS LAVSLGERAT INCKSSQSLL | 20 |
| | NSRTRKNYLA WYQQKPGQPP KLLIYWASTR | |
| | ESGVPDRFSG SGSGTDFTLT ISSLQAEDVA | |
| | VYYCTQSFIL RTFGQGTKVE IKRTVAAPSV | |
| | FIFPPSDEQL KSGTASVVCL LNNFYPREAK | |
| | VQWKVDNALQ SGNSQESVTE QDSKDSTYSL | |
| | SSTLTLSKAD YEKHKVYACE VTHQGLSSPV | |
| | TKSFNRGEC | |

In some embodiments, the anti-CD20/anti-CD3 bispecific antibody useful in the methods provided herein is glofitamab. Glofitamab (Proposed INN: List 121 WHO Drug Information, Vol. 33, No. 2, 2019, page 276, also known as CD20-TCB, R07082859, or RG6026) is a novel T-cell-engaging bispecific full-length antibody with a 2:1 molecular configuration for bivalent binding to CD20 on B cells and monovalent binding to CD3, particularly the CD3 epsilon chain (CD3ε), on T cells. Its CD3-binding region is fused to one of the CD20-binding regions in a head-to-tail fashion via a flexible linker. This structure endows glofitamab with superior in vitro potency versus other CD20-CD3 bispecific antibodies with a 1:1 configuration and leads to profound antitumor efficacy in preclinical DLBCL models. CD20 bivalency preserves this potency in the presence of competing anti-CD20 antibodies, providing the opportunity for pre- or co-treatment with these agents. Glofitamab comprises an engineered, heterodimeric Fc region with completely abolished binding to FcgRs and C1q. By simultaneously binding to human CD20-expressing tumor cells and to the CD3e of the T-cell receptor (TCR) complex on T-cells, it induces tumor cell lysis, in addition to T-cell activation, proliferation and cytokine release. Lysis of B-cells mediated by glofitamab is CD20-specific and does not occur in the absence of CD20 expression or in the absence of simultaneous binding (cross-linking) of T-cells to CD20-expressing cells. In addition to killing, T-cells undergo activation due to CD3 cross-linking, as detected by an increase in T-cell activation markers (CD25 and CD69), cytokine release (IFNγ, TNFα, IL-2, IL-6, IL-10), cytotoxic granule release (Granzyme B) and T-cell proliferation. The amino acid sequences for glofitamab are shown in Tables 2 and 3.

TABLE 2
Glofitamab amino acid sequences*

| | |
|---|---|
| Sequence | SEQ ID NO: |

CD20 VH-CH1(EE)-CD3 VL-CH1-Fc (knob, P329G LALA)

| | |
|---|---|
| QVQLVQSGAE VKKPGSSVKV SCKASGYAFS YSWINWVRQA PGQGLEWMGR | 21 |
| IFPGDGDTDY NGKFKGRVTI TADKSTSTAY MELSSLRSED TAVYYCARNV | |
| FDGYWLVYWG QGTLVTVSSA STKGPSVFPL APSSKSTSGG TAALGCLVED | |
| YFPEPVTVSW NSGALTSGVH TFPAVLQSSG LYSLSSVVTV PSSSLGTQTY | |
| ICNVNHKPSN TKVDEKVEPK SCDGGGGSGG GGSQAVVTQE PSLTVSPGGT | |
| VTLTCGSSTG AVTTSNYANW VQEKPGQAFR GLIGGTNKRA PGTPARFSGS | |
| LLGGKAALTL SGAQPEDEAE YYCALWYSNL WVFGGGTKLT VLSSASTKGP | |
| SVFPLAPSSK STSGGTAALG CLVKDYFPEP VTVSWNSGAL TSGVHTFPAV | |
| LQSSGLYSLS SVVTVPSSSL GTQTYICNVN HKPSNTKVDK KVEPKSCDKT | |
| HTCPPCPAPE AAGGPSVFLF PPKPKDTLMI SRTPEVTCVV VDVSHEDPEV | |
| KFNWYVDGVE VHNAKTKPRE EQYNSTYRVV SVLTVLHQDW LNGKEYKCKV | |
| SNKALGAPIE KTISKAKGQP REPQVYTLPP CRDELTKNQV SLWCLVKGFY | |
| PSDIAVEWES NGQPENNYKT TPPVLDSDGS FFLYSKLTVD KSRWQQGNVF | |
| SCSVMHEALH NHYTQKSLSL SP | |

CD20 VH-CH1(EE)-Fc (hole, P329G LALA)

| | |
|---|---|
| QVQLVQSGAE VKKPGSSVKV SCKASGYAFS YSWINWVRQA PGQGLEWMGR | 22 |
| IFPGDGDTDY NGKFKGRVTI TADKSTSTAY MELSSLRSED TAVYYCARNV | |
| FDGYWLVYWG QGTLVTVSSA STKGPSVFPL APSSKSTSGG TAALGCLVED | |
| YFPEPVTVSW NSGALTSGVH TFPAVLQSSG LYSLSSVVTV PSSSLGTQTY | |
| ICNVNHKPSN TKVDEKVEPK SCDKTHTCPP CPAPEAAGGP SVFLFPPKPK | |
| DTLMISRTPE VTCVVVDVSH EDPEVKFNWY VDGVEVHNAK TKPREEQYNS | |
| TYRVVSVLTV LHQDWLNGKE YKCKVSNKAL GAPIEKTISK AKGQPREPQV | |
| CTLPPSRDEL TKNQVSLSCA VKGFYPSDIA VEWESNGQPE NNYKTTPPVL | |
| DSDGSFFLVS KLTVDKSRWQ QGNVFSCSVM HEALHNHYTQ KSLSLSP | |

CD20 VL-CL(RK)

| | |
|---|---|
| DIVMTQTPLS LPVTPGEPAS ISCRSSKSLL HSNGITYLYW YLQKPGQSPQ | 23 |
| LLIYQMSNLV SGVPDRFSGS GSGTDFTLKI SRVEAEDVGV YYCAQNLELP | |
| YTFGGGTKVE IKRTVAAPSV FIFPPSDRKL KSGTASVVCL LNNFYPREAK | |
| VQWKVDNALQ SGNSQESVTE QDSKDSTYSL SSTLTLSKAD YEKHKVYACE | |
| VTHQGLSSPV TKSFNRGEC | |

CD3 VH-CL

| | |
|---|---|
| EVQLLESGGG LVQPGGSLRL SCAASGFTFS TYAMNWVRQA PGKGLEWVSR | 24 |
| IRSKYNNYAT YYADSVKGRF TISRDDSKNT LYLQMNSLRA EDTAVYYCVR | |
| HGNFGNSYVS WFAYWGQGTL VTVSSASVAA PSVFIFPPSD EQLKSGTASV | |
| VCLLNNFYPR EAKVQWKVDN ALQSGNSQES VTEQDSKDST YSLSSTLTLS | |
| KADYEKHKVY ACEVTHQGLS SPVTKSFNRG EC | |

Full-length Ab: HC-knob

| | |
|---|---|
| QVQLVQSGAE VKKPGSSVKV SCKASGYAFS YSWINWVRQA PGQGLEWMGR | 25 |
| IFPGDGDTDY NGKFKGRVTI TADKSTSTAY MELSSLRSED TAVYYCARNV | |
| FDGYWLVYWG QGTLVTVSSA STKGPSVFPL APSSKSTSGG TAALGCLVED | |
| YFPEPVTVSW NSGALTSGVH TFPAVLQSSG LYSLSSVVTV PSSSLGTQTY | |
| ICNVNHKPSN TKVDEKVEPK SCDGGGGSGG GGSQAVVTQE PSLTVSPGGT | |
| VTLTCGSSTG AVTTSNYANW VQEKPGQAFR GLIGGTNKRA PGTPARFSGS | |
| LLGGKAALTL SGAQPEDEAE YYCALWYSNL WVFGGGTKLT VLSSASTKGP | |
| SVFPLAPSSK STSGGTAALG CLVKDYFPEP VTVSWNSGAL TSGVHTFPAV | |
| LQSSGLYSLS SVVTVPSSSL GTQTYICNVN HKPSNTKVDK KVEPKSCDKT | |
| HTCPPCPAPE AAGGPSVFLF PPKPKDTLMI SRTPEVTCVV VDVSHEDPEV | |
| KFNWYVDGVE VHNAKTKPRE EQYNSTYRVV SVLTVLHQDW LNGKEYKCKV | |
| SNKALGAPIE KTISKAKGQP REPQVYTLPP CRDELTKNQV SLWCLVKGFY | |
| PSDIAVEWES NGQPENNYKT TPPVLDSDGS FFLYSKLTVD KSRWQQGNVF | |
| SCSVMHEALH NHYTQKSLSL SPGK | |

Full-length Ab: HC-hole

| | |
|---|---|
| QVQLVQSGAE VKKPGSSVKV SCKASGYAFS YSWINWVRQA PGQGLEWMGR | 26 |
| IFPGDGDTDY NGKFKGRVTI TADKSTSTAY MELSSLRSED TAVYYCARNV | |
| FDGYWLVYWG QGTLVTVSSA STKGPSVFPL APSSKSTSGG TAALGCLVED | |
| YFPEPVTVSW NSGALTSGVH TFPAVLQSSG LYSLSSVVTV PSSSLGTQTY | |
| ICNVNHKPSN TKVDEKVEPK SCDKTHTCPP CPAPEAAGGP SVFLFPPKPK | |
| DTLMISRTPE VTCVVVDVSH EDPEVKFNWY VDGVEVHNAK TKPREEQYNS | |
| TYRVVSVLTV LHQDWLNGKE YKCKVSNKAL GAPIEKTISK AKGQPREPQV | |
| CTLPPSRDEL TKNQVSLSCA VKGFYPSDIA VEWESNGQPE NNYKTTPPVL | |
| DSDGSFFLVS KLTVDKSRWQ QGNVFSCSVM HEALHNHYTQ KSLSLSPGK | |

LC-CD3

| | |
|---|---|
| EVQLLESGGG LVQPGGSLRL SCAASGFTFS TYAMNWVRQA PGKGLEWVSR | 27 |
| IRSKYNNYAT YYADSVKGRF TISRDDSKNT LYLQMNSLRA EDTAVYYCVR | |
| HGNFGNSYVS WFAYWGQGTL VTVSSASVAA PSVFIFPPSD EQLKSGTASV | |
| VCLLNNFYPR EAKVQWKVDN ALQSGNSQES VTEQDSKDST YSLSSTLTLS | |
| KADYEKHKVY ACEVTHQGLS SPVTKSFNRG EC | |

LC-CD20

| | |
|---|---|
| DIVMTQTPLS LPVTPGEPAS ISCRSSKSLL HSNGITYLYW YLQKPGQSPQ | 28 |
| LLIYQMSNLV SGVPDRFSGS GSGTDFTLKI SRVEAEDVGV YYCAQNLELP | |
| YTFGGGTKVE IKRTVAAPSV FIFPPSDRKL KSGTASVVCL LNNFYPREAK | |
| VQWKVDNALQ SGNSQESVTE QDSKDSTYSL SSTLTLSKAD YEKHKVYACE | |
| VTHQGLSSPV TKSFNRGEC | |

CD3 VH

| | |
|---|---|
| EVQLLESGGG LVQPGGSLRL SCAASGFTFS TYAMNWVRQA PGKGLEWVSR | 29 |
| IRSKYNNYAT YYADSVKGRF TISRDDSKNT LYLQMNSLRA EDTAVYYCVR | |
| HGNFGNSYVS WFAYWGQGTL VTVSSAS | |

CD3 VL

| | |
|---|---|
| QAVVTQEPSL TVSPGGTVTL TCGSSTGAVT TSNYANWVQE KPGQAFRGLI | 30 |
| GGTNKRAPGT PARFSGSLLG GKAALTLSGA QPEDEAEYYC ALWYSNLWVF | |
| GGGTKLTVLS S | |

CD20 VH

| | |
|---|---|
| QVQLVQSGAE VKKPGSSVKV SCKASGYAFS YSWINWVRQA PGQGLEWMGR | 31 |
| IFPGDGDTDY NGKFKGRVTI TADKSTSTAY MELSSLRSED TAVYYCARNV | |
| FDGYWLVYWG QGTLVTVSS | |

CD20 VL

| | |
|---|---|
| QVQLVQSGAE VKKPGSSVKV SCKASGYAFS YSWINWVRQA PGQGLEWMGR | 32 |
| IFPGDGDTDY NGKFKGRVTI TADKSTSTAY MELSSLRSED TAVYYCARNV | |
| FDGYWLVYWG QGTLVTVSS | |

*Residues that are underlined indicate the variable regions. Boldface residues indicate PGLALA constituents; Italicized residues indicate ‘charge modifications’.

TABLE 3
Glofitamab CDR sequences (Kabat)

| | | |
|---|---|---|
| | | SEQ ID |
| Description | Sequence | NO: |

CD20 heavy chain CDRs (Kabat)

| | | |
|---|---|---|
| HCDRI | YSWIN | 33 |
| HCDR2 | RIFPGDGDTDYNGKFKG | 34 |
| HCDR3 | NVFDGYWLVY | 35 |

CD20 light chain CDRs (Kabat)

| | | |
|---|---|---|
| LCDR1 | RSSKSLLHSNGITYLY | 36 |
| LCDR2 | QMSNLVS | 37 |
| LCDR3 | AQNLELPYT | 38 |

CD3 heavy chain CDRs (Kabat)

| | | |
|---|---|---|
| HCDRI | TYAMN | 39 |
| HCDR2 | RIRSKYNNYATYYADSVKG | 40 |
| HCDR3 | HGNFGNSYVSWFAY | 41 |

CD3 light chain CDRs (Kabat)

| | | |
|---|---|---|
| LCDR1 | GSSTGAVTTSNYAN | 42 |
| LCDR2 | GTNKRAP | 43 |
| LCDR3 | ALWYSNLWV | 44 |

The term “bispecific antibody treatment”, as used herein, refers to a treatment using a bispecific antibody.

The term “on-treatment” time period, as used herein, refers to a time period that begins once administration of a treatment (or a cycle of a treatment) has been initiated and that ends when administration of the treatment (or the cycle of the treatment) has concluded (potentially extended by a predefined buffer time interval). For example, an on-treatment time period may end 30 minutes after a conclusion of a treatment administration. The on-treatment time period may include a time during which the treatment (or the cycle of the treatment) is being infused to the subject. The on-treatment time period may be (for example) at least 15 minutes, at least 30 minutes, at least 1 hour, at least 2 hours, at least 3 hours, at least 4 hours, at least 6 hours, or at least 8 hours. The on-treatment time period may be (for example) less than 24 hours, less than 12 hours, less than 10 hours, less than 9 hours, less than 8 hours, less than 7 hours, or less than 6 hours. For example, the on-treatment time period may be between 3-5 hours long. As another example, the on-treatment time period may be between 7-9 hours long. The on-treatment time period includes multiple discrete on-treatment time points, such as a time point corresponding to a middle of a treatment administration and a time point corresponding to an end of a treatment administration.

The term “on-treatment level of a cytokine” or “on-treatment cytokine level”, as used herein, refers to a level of a particular cytokine detected in a biological sample (e.g., a blood sample or tissue sample) collected during the on-treatment time period. If multiple biological samples were collected from a given subject during the on-treatment time period and a cytokine level was determined for each sample, the on-treatment level of the cytokine can be defined to be a maximum of these cytokine levels. The on-treatment level of the cytokine can be determined using (for example) introducing capture and detection antibodies of the cytokine to the biological sample collected during the on-treatment time period.

The term “baseline” time period, as used herein, refers to a time period that ends with initiation of administration of a time period. The baseline time period may extend to and include a time point at which treatment is initiated. The baseline time period may include a time during which a pre-treatment is administered.

The term “baseline level of a cytokine” or “baseline cytokine level”, as used herein, refers to a level of a particular cytokine detected in a biological sample (e.g., a blood sample or tissue sample) collected during the baseline time period. The biological sample processed to identify the baseline level of the particular cytokine may include a sample collected at a predefined time before initiation of a treatment administration or within a predefined time interval before initiation of a treatment administration. The baseline level of the cytokine can be determined using (for example) introducing capture and detection antibodies of the cytokine to the biological sample collected during the baseline time period.

The term “baseline characteristic” of a subject includes a characteristic of a subject that was detected during a baseline time period, a characteristic that was detected before a baseline time period but presumed to be static, a characteristic that is static, or a characteristic that changes in a defined manner. For example, if a subject was diagnosed with a sub-type of a disease before the baseline time period but the baseline time period itself did not include any sub-type diagnosis, it may be presumed that the subject's disease remained as the same sub-type. Thus, the sub-type may be a baseline characteristic. As another example, a race of a subject may have been recorded before a baseline period, during a baseline period, or during an on-treatment period, but given that this type of characteristic is generally static throughout a person's life, the race may be characterized as a baseline characteristic regardless as to when it was recorded. Meanwhile, for a more dynamic variable (e.g., age), a baseline characteristic can be defined to be a value detected during the baseline time period and/or calculated based on a relative time of the baseline time period. A baseline characteristic may be based on an assessment of a sample collected during the baseline time period. For example, a baseline characteristic may characterize whether malignant cells are present and/or a degree to which malignant cells are present within a bodily component (from which a sample was collected). A baseline characteristic may be determined based on one or more images collected during the baseline time period. For example, a baseline characteristic may characterize a tumor burden or tumor spread based on computerized tomography (CT) images or other medical images. A baseline characteristic may include a static or changing demographic attribute and/or a comorbidity (e.g., indicating whether a subject has any comorbidities, whether the subject has a particular type of comorbidity, and/or what type of comorbidity a subject has).

The term “cytokine fold change”, as used herein, refers to a value calculated using at least two cytokine levels. The at least two cytokine values can include a baseline level of a cytokine and any other level of the cytokine (associated with a same subject). For example, the any other level of the cytokine can include another baseline level of the cytokine, an on-treatment level of the cytokine, or a level of the cytokine determined using a sample collected from the subject after the on-treatment time period. The cytokine fold change may be defined based on or defined to equal a log of the other level of the cytokine minus a log of the baseline level of the cytokine. The log may be of any positive base (e.g., log base 2 or log base 10).

The term “on-treatment cytokine fold change”, as used herein, refers to a cytokine fold change where the other level of the cytokine is an on-treatment level of the cytokine.

The term “cytokine release syndrome risk score”, as used herein, refers to a score (that is typically numeric but could be categorical) generated using one or more baseline characteristics that represents a predicted risk of a subject experiencing a cytokine release syndrome. The predicted risk may be of a subject experiencing a cytokine release syndrome of any grade, of at least a threshold grade (e.g., Grade 2 or higher), or of a particular grade. The predicted risk may be of a subject experiencing a cytokine release syndrome within a given time window, such as a time window that begins with an initiation or completion of a treatment administration and that has a duration of a predefined number of hours or days (e.g., 1 day, 2 days, 3 days, 5 days, 7 days or 14 days).

The term “cytokine release syndrome risk”, as used herein, refers to a score (that is typically categorical but could be numeric) derived from one or more cytokine values, a treatment dosage or exposure, and one or more risk scores.

The term “data record”, as used herein, refers to a collection of data associated with one or more indices. The one or more indices may correspond to (for example) an identification of a given subject, a given time, and/or a given time period. For example, a data record may include information about a particular subject was collected at a particular time point. A data record may include any collection of data that is retrievable by submitting a query that identifies a subject (and potentially one or more other constraints, such as a time point). For example, a data record may include a file, a row in a table, a column in a table, an element in an array, a subset of stored data that where all of the subset is associated with one or more indices, etc.

The terms “treatment dosage”, “dosage of a treatment”, or “dosage of at least part of a treatment”, as used herein, refer to a dosage of a treatment or of an active ingredient of a treatment. The dosage may be that administered with a cycle of the treatment (e.g., a first cycle) or across the entire treatment.

### III. Exemplary Network for Stratifying Subjects for Differential Monitoring by Predicting Cytokine Release Syndrome Risks

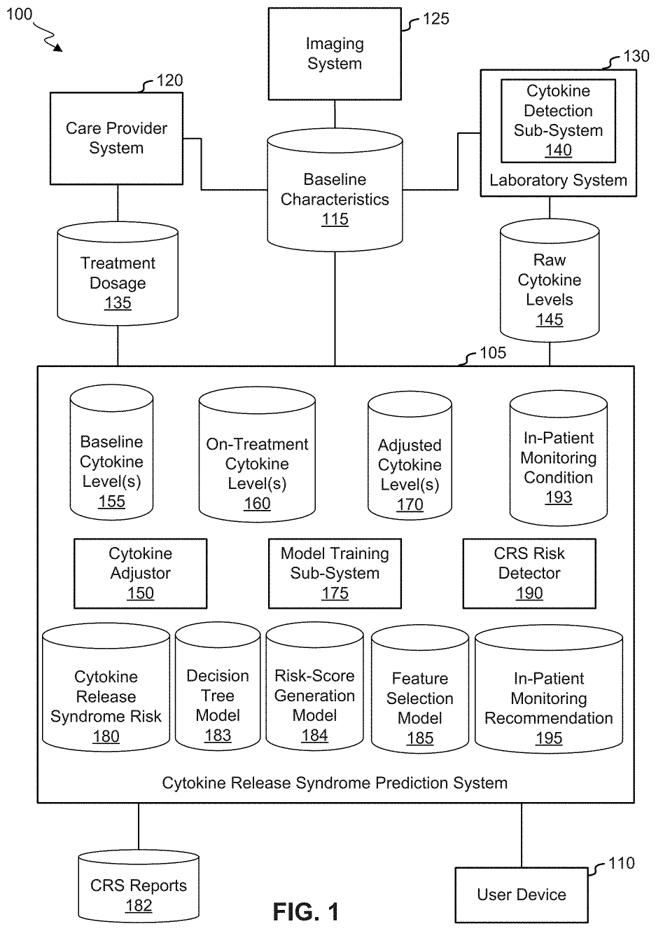

FIG. 1 shows an exemplary network 100 for stratifying subjects for differential monitoring or treatment by predicting a risk of one or more individual subjects experiencing a cytokine risk syndrome event according to some embodiments. Network 100 includes a cytokine release syndrome prediction system 105 that receives a request from a user device 110 to predict a risk that a particular subject will subsequently experience a cytokine release syndrome (e.g., of at least a particular grade and/or within a predefined time period). User device 110 may be operated by (for example) a physician, nurse, medical technician, or coordinator of a clinical study. The request may identify the particular subject by name and/or via one or more identifiers (e.g., a social security number or unique identifier). The request may identify a disease with which the particular subject has been diagnosed and/or a treatment that the particular subject has been prescribed and/or has received.

### III.A. Exemplary Subject Characteristics

The particular subject may have been diagnosed with cancer, such as non-Hodgkin's lymphoma.

### III.A.1. Non-Hodgkin's Lymphoma

Non-Hodgkin's lymphoma is a tissue and molecular malignancy that is the tenth most common cancer in the world. Annually, over 280,000 new cases of non-Hodgkin's lymphoma are diagnosed worldwide. The particular subject may reside in or may have been born in any geographic region. Although the incidence of non-Hodgkin's lymphoma varies by geographic region, the areas with the highest incidence of non-Hodgkin's lymphoma are North America, Europe and Australia, as well as several countries in Africa and South America. According to the American Cancer Society, non-Hodgkin's lymphoma is one of the most common cancers in the United States, accounting for about 4% of all cancers. In 2021, about 81,500 people in the United States will be diagnosed with non-Hodgkin's lymphoma, and about 20,720 people will die from this cancer.

The particular subject may be of any age, as non-Hodgkin's lymphoma can occur at any age. In fact, it is one of the more common cancers among children, teens, and young adults. Overall, the chance that a male will develop non-Hodgkin's lymphoma in his lifetime is about 1 in 41, For a female, the risk is about 1 in 53. However, each person's risk can be affected by a number of risk factors. Many people with non-Hodgkin's lymphoma have no obvious risk factors. It is also possible to have multiple risk factors and never develop non-Hodgkin's lymphoma. Some factors that may increase the risk of non-Hodgkin's lymphoma include: older age, since most people are aged 60 or older when diagnosed; the use of immunosuppressant drugs; an infection, particularly with HIV, Epstein-Barr virus, or Helicobacter pylori; and exposure to certain chemicals, such as weed and insect killers.

Non-Hodgkin lymphoma is the group name for all types of lymphomas except Hodgkin lymphoma. Non-Hodgkin's lymphoma is a diverse group of blood cancers that all arise from lymphocytes (white blood cells) that are part of the immune system. These cells are in the lymph nodes, spleen, thymus, bone marrow, and other parts of the body. Non-Hodgkin's lymphoma generally develops in the lymph nodes and lymphatic tissue found in organs such as the skin, stomach and intestines, with some cases exhibiting involvement in bone marrow and blood.

Non-Hodgkin's lymphoma develops when a cell in a lymph node or in another lymphatic structure undergoes a mutation. The disease can start in B lymphocytes (B cells), which produce antibodies to combat infections; T lymphocytes (T cells), which possess several functions, including assisting B lymphocytes with production of antibodies; or natural killer (NK) cells, which attack virus-infected cells or tumor cells, with approximately 85-90 percent of non-Hodgkin's lymphoma cases starting in the B cells of a subject. The mutated or abnormal lymphocytes exhibit uncontrolled growth and produce more abnormal cells that accumulate to form tumors. Eventually, if non-Hodgkin's lymphoma is left untreated, the abnormal cells (i.e., cancerous cells) crowd out normal white blood cells and the immune system cannot protect against infection effectively.

The early stages of non-Hodgkin's lymphoma are often asymptomatic. Therefore, regular medical check-ups are important for people with known risk factors for non-Hodgkin's lymphoma (such as HIV infections, organ transplants, autoimmune disease, or prior cancer treatment). These people do not often get lymphoma, but they and their doctors typically look out for possible symptoms and signs of lymphoma. One of the most common symptoms in subjects with non-Hodgkin's lymphoma is enlargement of one or more lymph nodes in the neck, armpit or groin. Occasionally, the disease starts in a site other than the lymph nodes, such as a bone, a lung, the gastrointestinal tract or the skin. In these circumstances, subjects may experience symptoms that are associated with that specific site. Although signs and symptoms will vary, common symptoms also include, unexplained fever, night sweats, persistent fatigue, loss of appetite, unexplained weight loss, cough or chest pain, abdominal pain, bloating, itchy skin, enlargement of the spleen or liver and rashes or skin lumps. The particular subject may have experienced or may be experiencing any one or more of the above symptoms.

### III.A.1.a. Diagnosis of Non-Hodgkin's Lymphoma

The particular subject may have been diagnosed with non-Hodgkin's lymphoma after the diagnosis was suspected (e.g., based on symptoms). The diagnosis can facilitate prescribing a treatment effective for managing the disease.

In addition to a physical examination, blood and urine tests may have been often performed to rule out infection or other disease. Imaging tests such as, for example, X-ray, CT, MRI or positron emission tomography (PET) may have been used to detect tumors throughout the body. A biopsy of an involved lymph node or other tumor site may have been used to confirm the non-Hodgkin's lymphoma diagnosis and subtype. Further testing may have included immunophenotyping or flow cytometry to identify specific types of cancer cells in a sample; cytogenetic analysis to look for chromosomal changes or abnormalities in the cells; and/or gene expression profiling to identify genes that were differentially expressed in the cancerous cells of the subject.

The particular subject may have been diagnosed with any kind of non-Hodgkin's lymphoma, such as one or more of the more than sixty subtypes of non-Hodgkin's lymphoma identified by the World Health Organization (WHO). These subtypes are categorized by the characteristics of the lymphoma cells, including their appearance, the presence of specific cell-surface proteins, and their genetic profile. Given that signs, symptoms and treatment for non-Hodgkin's lymphoma can vary depending on the subtype and rate of progression of the disease, accurate diagnosis and monitoring of disease progression are important for to identify a treatment to treat a given subtype and current progression for the particular subject.

Pathologists often describe non-Hodgkin's lymphoma in terms of grade. A high-grade lymphoma has cells that grow quickly and have morphologies different from normal cells. Low-grade lymphomas have cells that look much more like normal cells and multiply slowly. Intermediate-grade lymphomas fall somewhere in between. The behavior of these types is also described as indolent and aggressive.

When a pathologist describes a high-grade or intermediate-grade lymphoma, these types of lymphoma usually grow fast in the body, so these two types of lymphoma are considered aggressive lymphomas. Low-grade non-Hodgkin's lymphoma, on the other hand, grows slowly, and these lymphomas are called indolent lymphomas. Pathologists also classify the non-Hodgkin's lymphoma as a follicular or diffuse lymphoma. In follicular lymphoma, the cancer cells arrange themselves in spherical clusters called follicles. In diffuse non-Hodgkin's lymphoma, the cells are spread out without any clustering. Generally, low-grade non-Hodgkin's lymphoma or indolent non-Hodgkin's lymphoma, looks follicular, and intermediate or high-grade non-Hodgkin's lymphoma (aggressive non-Hodgkin's lymphoma) looks diffuse in biopsy slides.

Aggressive lymphomas account for about sixty percent of all non-Hodgkin's lymphoma cases, with diffuse large B-cell lymphoma (DLBCL) being the most common, aggressive non-Hodgkin's lymphoma subtype. Indolent lymphomas are slow-moving, tend to grow more slowly and have fewer signs and symptoms when first diagnosed. Low-grade or indolent subtypes represent about forty percent of all non-Hodgkin's lymphoma cases, with follicular lymphoma (FL) being the most common subtype of indolent non-Hodgkin's lymphoma. In some cases, indolent non-Hodgkin's lymphoma can transform into aggressive non-Hodgkin's lymphoma. When a subject's rate of disease progression is between indolent and aggressive, the subject is considered to have intermediate grade disease.

Table 4 provides some of the diagnostic designations for non-Hodgkin's lymphoma subtypes based on the WHO classification, categorized by cell type (B cell, T cell or NK cell) and rate of progression (aggressive or indolent). The percentages listed reflect the frequency of diagnosed cases of the most common non-Hodgkin's lymphoma subtypes.

TABLE 4
Diagnostic Designations for Non-Hodgkin's Lymphoma Sub Types
Mature B-cell lymphomas (about 85%-90% of non-Hodgkin's lymphoma cases)
Aggressive
Diffuse large B-cell lymphoma (DLBCL) (31%)
Mantle cell lymphoma (MCL) (can present as aggressive or indolent) (6%)
Lymphoblastic lymphoma (2%)
Burkitt lymphoma (BL) (2%)
Primary mediastinal (thymic) large B-cell lymphoma (PMBCL) (2%)
Transformed follicular and transformed mucosa-associated lymphoid tissue (MALT) lymphomas
High-grade B-cell lymphoma with double or triple hits (HBL)
Primary cutaneous diffuse large B-cell lymphoma, leg type
Primary diffuse large B-cell lymphoma of the central nervous system
Primary central nervous system (CNS) lymphoma
Acquired immunodeficiency syndrome (AIDS)-associated lymphoma
Indolent
Follicular lymphoma (FL) (22%)
Marginal zone lymphoma (MZL) (8%)
Chronic lymphocytic leukemia/small-cell lymphocytic lymphoma (CLL/SLL) (6%)
Gastric mucosa-associated lymphoid tissue (MALT) lymphoma (5%)
Lymphoplasmacytic lymphoma (1%)
Waldenstrom macroglobulinemia (WM)
Nodal marginal zone lymphoma (NMZL) (1%)
Splenic marginal zone lymphoma (SMZL)
Mature T-cell and natural killer (NK)-cell lymphomas (about 10%-15% of non-Hodgkin's
lymphoma cases)
Aggressive
Peripheral T-cell lymphoma (PTCL), not otherwise specified (6%)
Systemic anaplastic large-cell lymphoma (ALCL) (2%)
Lymphoblastic lymphoma (2%)
Hepatosplenic gamma/delta T-cell lymphoma
Subcutaneous panniculitis-like T-cell lymphoma (SPTCL)
Enteropathy-type intestinal T-cell lymphoma
Primary cutaneous anaplastic large-cell lymphoma
Angioimmunoblastic T-cell lymphoma (AITL)
Indolent
Cutaneous T-cell lymphoma (CTCL) (4%)
Mycosis fungoides (MF)
Sezary syndrome (SS)
Adult T-cell leukemia/lymphoma
Extranodal NK/T-cell lymphoma (ENK/TCL), nasal type

The particular subject may have been diagnosed with and/or may have any of the lymphoma sub-types shown in Table 4. The particular subject may have been diagnosed with non-Hodgkin's lymphoma after the diagnosis was suspected (e.g., based on symptoms). The diagnosis can facilitate prescribing a treatment effective for managing the disease.

Diagnosis can also include non-Hodgkin's lymphoma grading or staging to identify the location of the cancer, the number of lymph nodes affected the cancer and whether the disease has spread from the original site to other parts of the body, for example, to the liver or lungs. The majority of lymphomas are nodal lymphomas, i.e., they originate in the lymph nodes. However, lymphomas can arise anywhere in the human body. When the lymphoma is mainly present in the nodes, it is referred to as nodal disease. Occasionally, most of the lymphoma may be in an organ that is not a part of the lymph system, for example, the stomach, the skin or the brain. In these instances, the lymphoma is referred to as extranodal. Nodal and extranodal refer to the primary site of the disease. A lymphoma can develop in a lymph node and subsequently involve other structures. In these cases, it is referred to as a nodal lymphoma with extranodal involvement.

The particular subject may have been assigned a grade of non-Hodgkin's lymphoma based on the following definition of various stages:
Stage I: The cancer is found in a single region or organ, usually one lymph node and the surrounding area.
Stage II: The cancer is found in two or more lymph node regions on the same side of the diaphragm, either above or below it.
Stage III: The cancer is found in lymph nodes on both sides of the diaphragm. If the cancer is also outside the lymph system, it is called stage IIIE. Stage III lymphoma that is also in the spleen is stage IIIS. If it is stage IIIS and has spread outside the lymph systems, it is stage IIIE+S.
Stage IV: The cancer has spread to one or more tissues or organs outside the lymph system, such as the liver, lungs or bones, and may be found in lymph nodes near or far away from those organs.
Stage V: Death.

### III.A.1.b. Treatment of Non-Hodgkin's Lymphoma

The particular subject may have been prescribed to receive or may have already received a treatment that has a potential of triggering a cytokine release syndrome. The treatment may include (for example) a treatment identified in Section III.A.1.b.i. or III.A.1.b.ii. below. The particular subject may have further been prescribed to receive or may have already received a pre-treatment before the treatment is administered. A composition of and/or an active agent within the pre-treatment may be the same or different from that of the treatment.

Treatment for non-Hodgkin's lymphoma can depend on the non-Hodgkin's lymphoma subtype, rate of progression, and/or stage of the disease. Lymphomas that do not cause signs and symptoms may not require treatment for years. In some cases, if the initial cancer is small, the tumor can be removed during a biopsy, and no further treatment may be provided. However, if the non-Hodgkin's lymphoma is aggressive or causes signs and symptoms, treatment is often prescribed.

Treatment for indolent non-Hodgkin's lymphoma can range from a wait-and-see approach to aggressive therapy.

### III.A.1.b.i. Indolent Sub-Types

The particular subject may have been diagnosed with an indolent sub-type of non-Hodgkin's lymphoma (e.g., follicular lymphoma). Indolent non-Hodgkin's lymphoma management depends on prognostic factors, disease stage, age, and other medical conditions. Follicular lymphoma, the most common type of indolent non-Hodgkin's lymphoma, is a very slow growing disease. Treatment for some subjects may not be recommended for several years, whereas others may have extensive lymph node or organ involvement and thus have immediate treatment recommendations. In a small percentage of subjects, follicular lymphoma may transform into a more aggressive disease.

Grade 1 or grade 2 follicular lymphoma may be treated with a watch-and-wait approach that involves periodic examinations and imaging tests or radiation therapy. Radiation therapy is most often used to treat early-stage non-Hodgkin's lymphoma, where the cancer is only in one part of the body. Treatment is normally given in short, daily sessions, usually for no longer than three weeks. In some instances, early stage, indolent non-Hodgkin's lymphoma can be treated with chemotherapy, chemotherapy in combination with radiotherapy, or chemotherapy in combination with immunotherapy, for example, monoclonal antibody therapy. Rituximab (Rituxan®)(Genentech, San Francisco, Calif.) is a monoclonal antibody used to treat many different types of B-cell non-Hodgkin's lymphoma. Rituximab works by targeting CD20, on the surface of all B cells and B-cell non-Hodgkin's lymphoma. When the antibody attaches to CD20 on B cells, the subject's immune system is activated to destroy some lymphoma cells or to make lymphoma cells more susceptible to being destroyed by chemotherapy. Although rituximab may work well by itself, research shows that it works better when added to chemotherapy for subjects with most types of B-cell non-Hodgkin's lymphoma. Rituximab is also given after remission for indolent lymphoma to increase the length of the remission. There are other monoclonal antibodies against CD20 that are approved by the FDA for use in lymphomas: obinutuzumab (Gazyva®), ofatumumab (Arzerra®), rituximab-abbs (Truxima®), rituximab-arrx (Riabni®), and rituximab-pvvr (Ruxience®).

In addition to classifying lymphoma by grade, some subjects are also classified as having relapsed or recurring follicular lymphoma. The Follicular Lymphoma International Prognostic Index (FLIPI) is a scoring system used to predict which subjects with follicular lymphoma may be at higher risk for disease recurrence. One point is assigned for each of the following risk factors (known by the acronym NoLASH):
Nodes involved—5 or more
Lactate dehydrogenase (LDH) level—higher than the upper limit of normal
Age older than 60 years
Grade 3 or grade 4 disease
Hemoglobin concentration—less than 12 g/dL

Risk is classified as follows: Low risk: 0 to 1 point; Intermediate risk: 2 points; and High risk: 3 to 5 points.

For subjects with grade 2 follicular lymphoma who have large lymph nodes, grade 3 follicular lymphoma, or grade 4 follicular lymphoma or advanced-stage relapsed follicular lymphoma, treatment will be based on symptoms, the subject's age and health status, the extent of disease and the subject's choice. Other treatment options include radiation therapy to lymph nodes that are causing symptoms, or to a large localized mass, if present; or chemotherapy (either as a single chemotherapeutic drug or as a chemotherapeutic combination), together with immunotherapy (rituximab).

Chemotherapeutic agents include, but are not limited to, alkylating agents (for example, cyclophosphamide, chlorambucil, bendamustine, ifosfamide), platinum drugs (for example, cisplatin, carboplatin and oxaliplatin), purine analogs (for example, cytarabine (ara-C), gemcitabine, methotrexate, pralatrexate); anthracyclines (for example, doxorubicin or liposomal doxorubicin), vincristine, mitoxantrone, etoposide (VP-16), and bleomycin. Often drugs from different groups are combined. One of the most common combinations is called CHOP, which includes the cyclophosphamide, doxorubicin (also known as hydroxydaunorubicin), vincristine (Oncovin®) and prednisone. Another common combination, CVP, does not include doxorubicin. CHOP or CVP can be administered in combination with rituximab (CHOP-R or CVP-R).

Some subjects with grade 2 follicular lymphoma who have large lymph nodes, grade 3, grade follicular lymphoma, or advanced-grade relapsed follicular lymphoma can be treated with stem cell transplantation (autologous and allogeneic) or targeted therapy with kinase inhibitors (for example, idelalisib (Zydelig®), copanlisib (Aliqopa®) and duvelisib (Copiktra™); lenalidomide (Revlimid®); or tazemetostat (Tazverik™).

A treatment that the subject is to receive or has received may include a bispecific antibody. A bispecific antibody may be provided or recommended as an immunotherapeutic to subjects with refractory or relapsed follicular lymphoma. Bispecific T-cell engaging antibodies (BiTE) and knobs-into-holes (KIHs) bispecific antibodies are exemplary antibody-based molecules engineered to bind two different epitopes, where one targets the malignant cells and the other one targets effector cells, usually T-lymphocytes, which mediate tumor cell destruction. Mosunetuzumab (Genentech), a T cell-dependent bispecific and glofitamab (Genentech), a KIH T cell bispecific, both specifically binding CD20 and CD3, both T-cell engaging bispecific antibodies, can be used to treat multiple types of non-Hodgkin's lymphoma, including relapsed follicular lymphoma and diffuse large B-cell lymphoma.

Glofitamab (also known as RO7082859, RG6026) and mosunetuzumab are investigational, full-length, CD20- and CD3-targeting T-cell specific antibodies that are designed to redirect T cells to engage and eliminate malignant B cells (Bacac et al. Clin. Cancer Res. doi:10.1158/1078-0432.CCR-18-0455; Sun et al. Science Translational Medicine 7(287):287ra70; DOI:10.1126/scitranslmed.aaa4802). These antibodies are designed to bind to CD20, a B-cell surface protein expressed in a majority of B-cell malignancies, while simultaneously binding to CD3, a component of the T-cell receptors on the surface of T cells. T-cell directed therapies that induce potent immune stimulation, pose the risk of cytokine release syndrome, potentially limiting their dose and utility. Glofitamab and mosunetuzumab contain targeted mutations of the Fc binding sites to mitigate unwanted lysis of attracted T cells, and off-target toxicity (e.g., cytokine release syndrome).

Follicular lymphoma has a small risk of transforming into an aggressive large B-cell lymphoma, such as diffuse large B-cell lymphoma. The particular subject may have been diagnosed with aggressive large B-cell lymphoma (e.g., after previously being diagnosed with follicular lymphoma).

Subjects with transformed B-cell follicular lymphoma can benefit from rituximab therapy, either alone or in combination with chemotherapy. Other options include axicabtagene ciloleucel (Yescarta®) and tisagenlecleucel (Kymriah®), both of which are CAR T-cell therapies. In a typical, CAR-T cell therapeutic protocol, T cells are collected from the blood of the subject and modified so that the T cells produce chimeric antigen receptors (CARs) on their surface. These CAR-T cells are reinfused into the subject where the CARs bind to a specific antigen on the subject's tumor cells and kill the tumor cells. See, for example, Lulla et al. “The Use of Chimeric Antigen Receptor T Cells in Patients with Non-Hodgkin Lymphoma, Clin. Adv. Hematol. Oncol. 16(5): 375-386 (2018)). As set forth above, bispecific antibody therapy, for example, glofitamab or mosunetuzumab can also be used to treat diffuse large B-cell lymphoma.

Cutaneous T-cell lymphomas (CTCLs) are a group of indolent non-Hodgkin's lymphomas that make up about 4% of non-Hodgkin's lymphoma cases. CTCLs develop primarily in the skin and may grow to involve lymph nodes, blood and other organs. Mycosis fungoides is the most common type of CTCL and is characterized by prominent skin involvement. When the malignant lymphocytes enter and accumulate in the blood, the disease is called Sézary syndrome. Therapy for CTCL depends on the nature of the skin lesions and whether disease is present in the lymph nodes.

Topical therapies are often used to treat the skin lesions. These include drugs applied directly to the skin as well as exposure of skin lesions to light via ultraviolet light therapy or electron beam therapy. A combination therapy that uses ultraviolet light, in conjunction with psoralen (a drug that is activated upon exposure to light) (PUVA), is also used. If there is widespread involvement of lymph nodes and other areas, chemotherapy or extracorporeal photopheresis can be used. Photopheresis is a process in which white blood cells are removed by apheresis, treated with psoralen, exposed to ultraviolet A light and then returned to the subject's bloodstream.

Administration of histone deacetylase (HDAC) inhibitors (romidepsin (Istodax®), given by IV infusion and vorinostat (Zolinza®), given by mouth)), as well as a monoclonal antibody (mogamulizumab (Poteligeo®), given by IV), are indicated for the treatment of adult subjects with either relapsed or refractory disease who have received previous systemic therapy.

### III.A.1.b.ii. Aggressive Sub-Types

Subjects with aggressive non-Hodgkin's lymphoma are frequently treated with chemotherapy that consists of four or more drugs. In most cases, this is CHOP or R-CHOP combination therapy described above. This intensive, multidrug chemotherapy can be very effective for aggressive lymphoma, and cures have been achieved. Chemotherapy can be supplemented by radiation therapy in select cases, for instance, when large non-Hodgkin's lymphoma masses are found during the diagnostic and staging process.

Although there are many types of aggressive non-Hodgkin's lymphoma, diffuse large B-cell lymphoma is the most common non-Hodgkin's lymphoma subtype, making up about 31 percent of all non-Hodgkin's lymphoma cases in the United States. It grows rapidly in the lymph nodes and frequently involves the spleen, liver, bone marrow or other organs. Usually, diffuse large B-cell lymphoma development starts in lymph nodes in the neck or abdomen and is characterized by masses of large B cells. In addition, subjects with diffuse large B-cell lymphoma often experience B symptoms (fever, night sweats and loss of more than 10 percent of body weight over 6 months). For some subjects, diffuse large B-cell lymphoma may be the initial diagnosis. For other subjects, an indolent lymphoma such as small lymphocytic lymphoma or follicular lymphoma transforms and becomes diffuse large B-cell lymphoma. Treatments include CHOP, dose adjusted EPOCH-R (dose-adjusted etoposide, prednisone, vincristine (Oncovin®), cyclophosphamide, hydroxydoxorubicin (doxorubicin) plus rituximab, and rituximab and hyaluronidase human (Rituxan Hycela™). Bispecific antibody therapy, for example, glofitamab or mosunetuzumab, can also be used to treat diffuse large B-cell lymphoma.

Some types of aggressive non-Hodgkin's lymphoma do not respond to standard doses of chemotherapy or have a high risk of recurrence. Doctors may consider giving a higher dose of chemotherapy followed by a stem cell transplant to treat some of these cases. In some instances, relapsed diffuse large B-cell lymphoma can be treated via CAR-T cell therapy, for example, with Yescarta®, Kymriah® or Breyanzi (lisocabtagene maraleucel). Axicabtagene ciloleucel (Yescarta®) is a CAR T-cell therapy that is approved to treat subjects with diffuse large B-cell lymphoma who have received at least 2 previous types of treatment. Tisagenlecleucel (Kymriah®) is another CAR T-cell therapy that is approved for the treatment of refractory B-cell lymphoma, including diffuse large B-cell lymphoma, after 2 previous systemic treatments or more. Further CAR T-cell therapies are in development and being studied in clinical trials. Lisocabtagene maraleucel (Breyanzi®) is a CAR T-cell therapy approved for the treatment of adults with recurrent or refractory large B-cell lymphoma after 2 or more lines of systemic therapy. It can be used to treat diffuse large B-cell lymphoma, not otherwise specified; high-grade B-cell lymphoma; primary mediastinal large B-cell lymphoma; and follicular lymphoma.

Polatuzumab vedotin-piiq (Polivy®) is a monoclonal antibody that targets CD79b. Polatuzumab is used in combination with bendamustine and rituximab to treat diffuse large B-cell lymphoma that has come back after at least 2 other treatments.

Tafasitamab-cxix (Monjuvi®) is a monoclonal antibody that targets the CD19 molecule. It can be used in combination with lenalidomide to treat recurrent or refractory diffuse large B-cell lymphoma in those who cannot receive an autologous bone marrow/stem cell transplant.

Burkitt lymphoma is an aggressive B-cell subtype that grows and spreads very quickly. It may involve the jaw, bones of the face, bowel, kidneys, ovaries, bone marrow, blood, central nervous system (CNS) and other organs. Burkitt lymphoma may spread to the brain and spinal cord (part of the CNS); therefore, treatment to prevent spread of Burkitt lymphoma is frequently included in any treatment regimen. Doctors typically use highly aggressive chemotherapy to treat this subtype of non-Hodgkin's lymphoma. Commonly used regimens include: CODOX-M/IVAC (cyclophosphamide, vincristine (Oncovin®), doxorubicin and high-dose methotrexate) alternating with IVAC (ifosfamide, etoposide and high dose cytarabine); hyper-CVAD (hyperfractionated cyclophosphamide, vincristine, doxorubicin (Adriamycin®) and dexamethasone) alternating with methotrexate and cytarabine). In small studies, rituximab was used in combination with hyper-CVAD; and DA-EPOCH-R (dose-adjusted etoposide, prednisone, vincristine (Oncovin®), cyclophosphamide, doxorubicin plus rituximab).

Mantle cell lymphoma (MCL), which can present as aggressive or indolent non-Hodgkin's lymphoma, originates from a lymphocyte in the mantle zone of the lymph node, and represents about 6% of non-Hodgkin's lymphoma cases. It begins in the lymph nodes and spreads to the spleen, blood, bone marrow and sometimes the esophagus, stomach and intestines. Some subjects do not show signs or symptoms of the disease, so delaying treatment may be an option for them. However, most subjects need to start treatment after diagnosis. The standard treatment is a combination chemotherapy regimen, either with or without an autologous stem cell transplant. Common treatment regimens include bendamustine plus rituximab; a form of CHOP in which bortezomib is used instead of vincristine. The following agents are indicated for relapsed and refractory MCL: acalabrutinib (Calquence®), given by mouth; bortezomib (Velcade®), given by IV or subcutaneous injection; ibrutinib (Imbruvica®), given by mouth; zanubrutinib (Brukinsa™), given by mouth; and lenalidomide (Revlimid®), given by mouth. Allogeneic transplantation with a standard or reduced-intensity conditioning regimen may be considered for subjects with relapsed and refractory MCL who achieve remission following second-line therapy. Brexucabtagene autoleucel (Tecartus®) is approved for adults with recurrent or refractory mantle cell lymphoma.

Peripheral T-cell lymphomas (PTCL) are a group of rare, aggressive non-Hodgkin lymphomas that develop from mature T cells and natural killer (NK) cells. They account for approximately 10 percent of non-Hodgkin's lymphoma cases. PTCL, not otherwise specified (PTCL NOS) is the most common subtype of PTCL, accounting for about thirty percent of PTCL cases. For most subtypes of PTCL, the initial treatment is typically a combination chemotherapy regimen, such as CHOP, CHOEP (etoposide, vincristine, doxorubicin, cyclophosphamide, and prednisone), or other multidrug regimens. Because most subjects with PTCL will relapse, some physicians recommend high-dose chemotherapy followed by an autologous stem cell transplant. For CD30-expressing PTCLs, brentuximab vedotin (Adcetris®) is approved for use in combination with cyclophosphamide, doxorubicin, and prednisone as initial treatment. Brentuximab vedotin is another type of monoclonal antibody, called an antibody-drug conjugate. Antibody-drug conjugates attach to targets on cancer cells and then release a small amount of chemotherapy or other toxins directly into the tumor cells. Brentuximab vedotin combined with chemotherapy is approved to treat adults with certain types of peripheral T-cell lymphoma, such as peripheral T-cell lymphoma, not otherwise specified, as long as they express the CD30 protein.

### III.A.1.c. Side Effects of Treatment for Non-Hodgkin's Lymphoma

Each type of treatment for non-Hodgkin's lymphoma has a different set of possible side effects, which can range from mild to severe. Common side effects associated with immunotherapy, chemotherapy, radiation therapy or combinations thereof, include anemia (low red blood cells), thrombocytopenia (low platelets), neutropenia (low white blood cells), risk of infection, nausea, vomiting, bowel problems, fatigue, brain fog, hair loss, peripheral neuropathy, dry skin, oral mucositis, sleep disorders, early menopause, and reduced fertility. Immunotherapy, in particular, can trigger more severe side effects such as, lung inflammation, diabetes, hypophysitis (pituitary inflammation), or cytokine release syndrome. Therefore, care providers typically carefully monitor for cytokine release syndrome in any subject with non-Hodgkin's lymphoma that has received immunotherapy, particularly bispecific T-cell engaging antibodies or CAR-T cell therapy.

### III.B. Exemplary Primary Sources of Baseline Characteristics

Cytokine release syndrome prediction system 105 can request and/or retrieve information about the particular subject from one or more sources (e.g., one or more data stores or one or more computing systems). For example, cytokine release syndrome prediction system 105 can retrieve a set of baseline characteristics of the subject from a baseline characteristics data store 115. (It will be appreciated that, while FIG. 1 depicts baseline characteristics data store 115 as a single data store, baseline characteristics may instead be stored in and retrieved from multiple separate baseline characteristics data stores 115.) Each baseline characteristic includes a characteristic of a subject that was detected during a baseline time period, a characteristic that was detected before a baseline time period but presumed to be static, a characteristic that is static, or a characteristic that changes in a defined manner. A baseline characteristic may have been determined based on data received from a care provider system 120, an imaging system 125 or a laboratory system 130. Each baseline characteristic may be stored within a baseline-characteristic data record, which can be stored in baseline-characteristics data store 115. Each baseline-characteristic data record can be associated with a particular subject. In some instances, a baseline-characteristic data record is associated with a particular time at which the particular subject is characterized via the baseline characteristic.

### III.B.1. Care Provider System

Care provider system 120 can include one or more computing systems that detect subject data representing: one or more past or current characteristics of the particular subject, one or more past or current medical assessments of the particular subject, a specification of one or more treatments previously prescribed for or administered to the particular subject, one or more medically related events experienced by the particular subject.

A past or current characteristic of the particular subject may identify (for example) a demographic characteristic (e.g., age, race, sex), a geographical characteristic (e.g., residential city), an occupational characteristic (e.g., identifying a current or previous profession), a current or previous symptom, medical-history information (e.g., one or more previous diagnoses, previous adverse event, a comorbidity as self-reported by the particular subject, and/or a family history pertaining to one or more types of diseases. A past or current medical assessment of the particular subject may include (for example) an existing or new diagnosis (e.g., identifying of a disease, a stage of disease, a sub-type of a disease), results of an in-office evaluation (e.g., assessing how well a given task was performed, whether any medical abnormalities were observed, vital signs, etc.), and/or a comorbidity as diagnosed by a medical professional. The medical assessment may have been performed by (for example) a physician or nurse associated with a same or different care provider system 120. A specification of a previous treatment may include an identification of a medication previously administered to the particular subject, an indication as to when the medication was administered (e.g., identifying one or more dates or one or more years), one or more dosages of the medication, a route of administration, and/or a treatment schedule (e.g., identifying how many dosages were received and relative timing of the dosages). A medically related event experienced by the particular subject may include a symptom, an adverse event, a surgical procedure, a hospitalization.

Some or all of the subject data may be detected at care provider system 120 by processing input received via an input component of care provider system 120. An input component may include keyboard, camera, scanner, microphone, mouse, track pad, etc. The input may (for example) correspond to medical notes from a care provider, a form completed by the particular subject, an order for a prescription from a care provider, etc. Additionally or alternatively, some or all of the subject data may be pulled from an electronic health record.

Subject data that was detected during a baseline time period, that was detected before a baseline time period but presumed to be static, that is static, or that changes in a defined manner are baseline characteristics and can be stored in baseline characteristics data stores 115. The baseline characteristics can be stored in association with an identifier of the particular subject.

Care provider system 120 may further identify one or more specifications of a treatment that is currently prescribed to or being administered to the particular subject. The one or more treatment specifications can identify a medication, a dosage, a route of administration, and/or a treatment schedule. The one or more treatment specifications can identify a pre-treatment agent, a dosage of a pre-treatment, or a timing of the pre-treatment (relative to a first treatment dosage). The pre-treatment agent may include an agent that is not a CD3 bispecific antibody. For example, the pre-treatment agent may include obinutuzumab.

In instances where multiple different dosages of a medication (e.g., of a CD3 bispecific antibody) are administered during a treatment course, a treatment schedule may identify at which relative times the different dosages are to be administered. For example, a set of treatment specifications may specify that 10 mg glofitamab are to be administered on a first treatment day and 16 mg glofitamab are to be administered 27 days after the first treatment day. In instances where multiple different medications are administered during a treatment course, a treatment schedule may identify at which relative times the different medications are to be administered. For example, a set of treatment specifications may specify that 10 mg glofitamab are to be administered on a first treatment day and that a combination of 10 mg glofitamab and 1000 mg obinutumzumab are to be administered on each of 16 and 35 days after the first treatment day. One or more treatment dosages can be stored in a treatment dosage data store 135 in association with an identifier of the particular subject. In some instances, a treatment specification (stored in treatment dosage data store 135) can further identify a time at which a treatment (or a corresponding pre-treatment) was initiated.

### III.B.2 Imaging System

Imaging system 125 includes one or more computing systems that collect and/or assess medical images. A medical image may be (for example) a computerized tomography (CT) image, an x-ray, a magnetic resonance imaging (MRI) scan, a positron emission tomography (PET) scan, or a digital pathology image. Thus, the medical image may have been collected using (for example) a CT machine, x-ray machine, MM machine, PET machine, or microscope. In some instances, imaging system 125 includes the machine or device that collects the medical image. In some instances, the medical image is collected using a remote imaging machine or device and is transmitted to imaging system 125 (e.g., in response to imaging system 125 sending a request for the image).

The medical image (e.g., a CT image, x-ray, Mill scan, or PET scan) may have been collected by imaging a portion of the particular subject, potentially after a contrast agent was administered to the particular subject. The medical image may be a two-dimensional image or a three-dimensional image. In some instances, multiple two-dimensional images are collected. The medical image(s) can be processed using a computer-vision algorithm (e.g., executed at imaging system 125) or based on annotations from a human annotator (e.g., detected by imaging system 125) to identify one or more tumor annotations. Each tumor annotation can identify a portion of the medical image(s) that depicts a part of a tumor. For example, imaging system 125 may provide an interface that depicts a medical image, and imaging system 125 may receive annotation data that indicates which parts of the medical image were identified by an annotator (via input) as being a boundary of a tumor in an image displayed by imaging system 125). Imaging system 125 can identify one or more spatial metrics for each identified tumor. A spatial metric may include (for example) a volume of the tumor, an area of the tumor, a length along a longest axis of the tumor (referred to as a longest diameter), and/or an aspect ratio of the tumor.

Imaging system 125 may further automatically detect (e.g., using a computer-vision algorithm) and classify each depiction of an organ or may receive input from an annotator that identifies a boundary of each depicted organ. Imaging system 125 can then use the annotation of a given tumor and of the organs to detect in which type of organ a tumor is located.

Imaging system 125 may generate tumor-characterizing statistics, such as a total quantity of detected tumors, a total volume of tumors (summed across detected tumors), an average of the tumors' longest diameters, a number of organ types in which at least one tumor was detected, a tumor burden, and/or or a sum of products of longest overall tumor diameters across tumors.

A tumor-characterizing statistic can be characterized as a baseline characteristic (stored in baseline characteristic data store 115) when the medical image(s) were collected during a baseline time period. In some instances, a baseline characteristic (that is then stored in baseline characteristic data store 115) is defined based on a numeric tumor-characterizing statistic. For example, a numeric tumor-characterizing statistic can be compared to one or more thresholds to generate a binary indicator as to whether the statistic exceeds a single threshold. As another example, a numeric tumor-characterizing statistic can be compared to multiple thresholds to identify one or multiple ranges that includes the statistic, and a categorical indicator can identify the category.

In some instances, a medical image is used to detect a size of lymph nodes. Enlarged lymph nodes can be indicative of lymphoma. Thus, a baseline statistic may be defined to be an estimated volume, estimated cross section, or estimated longest diameter of a lymph node.

Alternatively, the medical image (e.g., a digital pathology image) may have been collected by collecting a sample from the particular subject (e.g., a biopsy, tissue sample and/or blood sample), fixing the sample, potentially slicing the sample or dropping a liquid sample on a slide, and staining a slice of the sample. Imaging system 125 may then image the stained slice, or a remote imaging system may have imaged the stained slice and imaging system 125 may access the image(s).

Imaging system 125 may process the image(s) to detect the presence, location and/or density of any biological object of a given type (e.g., a particular cell type). For example, imaging system 125 may detect a point location (or area or volume) of each tumor cell and/or of each immune cell. Imaging system 125 may define a baseline characteristic (and store the baseline characteristic in baseline characteristic data store 115) to indicate a binary presence of any tumor cell, a density of tumor cells, a density of immune cells, etc.

### III.B.3. Laboratory System

Laboratory system 130 can process a biological sample to generate one or more laboratory results. Each laboratory result may identify a presence of, count of, concentration of, and/or type of each of one or more biological structures. The biological sample may be different than any sample used to collect a medical image (that is processed by imaging system 125). The biological sample may include (for example) a blood sample, urine sample, sweat sample, or tissue sample.

The biological structure (being measured by laboratory system 130) may include a cell type, cell fragment, or protein. For example, the biological structure may include white blood cells, monocytes, platelets, hemoglobin, fibrinogen, C-reactive protein (CRP), aspartate aminotransferase (AST), and/or alkaline phosphatase (ALP). High white blood cell counts, high monocyte counts, low platelet counts, may be consistent with various types of cancer (e.g., lymphoma). Low hemoglobin levels may be consistent with certain types of cancer (e.g., non-Hodgkin's lymphoma) or with an advanced stage of certain types of cancer (e.g., stage III or stage IV of Hodgkin's lymphoma). A high level of fibrinogen and/or C-reactive protein may be indicative of inflammation. A high level of AST and/or ALP may be indicative that a cancer (e.g., non-Hodgkin's lymphoma) has spread to the liver.

When the biological sample was collected during a baseline time period, the laboratory result(s) can be characterized as baseline characteristics and stored in baseline characteristic data store 115.

Laboratory system 130 includes a cytokine detection sub-system 140 that monitors a level of (e.g., concentration of) of each of one or more cytokines in the biological sample (or a different biological sample). Laboratory system 130 stores each cytokine level in a raw cytokine level data store 145, in association with a subject identifier, measurement time, and/or a cytokine identifier. For example, a single cytokine-level data record may be generated to correspond to an individual measurement time and to an individual subject and may include a level of each cytokine detected in a sample collected from the subject at the measurement time. As another example, a single cytokine-level data record may be generated to correspond to an individual subject and may include a level of each cytokine detected in any sample collected from the subject. The single cytokine-level data record may associate each level of a cytokine with a measurement time that indicates when a sample was collected from the subject that was used to measure the cytokine level. Each measurement time may be an absolute time or a time relative to a beginning of a time period for a given treatment.

As one example, cytokine detection sub-system 140 can detect a level of each of one or more of the following cytokines in a blood sample: IL-10, IL-2, IL-6, IL-8, MIP1b, MCP1, IL-10, IFN-γ, TGF-β, and TNF-α.

### III.C. Exemplary Cytokine Release Syndrome Prediction System

Cytokine release syndrome prediction system 105 can process one or more baseline characteristics (from baseline characteristics data store 115), one or more treatment dosages (from treatment dosage data store 135), and one or more cytokine levels (from raw cytokine levels data store 145) using a machine learning model to predict a risk that the particular subject will experience a cytokine release syndrome.

### III.C.1. Cytokine Release Syndrome

Cytokine release syndrome is an uncontrolled inflammatory response that can be triggered when treating non-Hodgkin's lymphoma, particularly when treating non-Hodgkin's lymphoma with therapeutic antibodies, CAR-T cell therapy or allogeneic transplantation. Cytokine release syndrome can occur after infusion of any of several antibody-based therapies, such as glofitamab, rituximab, obinutuzumab, alemtuzumab, brentuximab, dacetuzumab, or nivolumab. Cytokine release syndrome has also been observed following administration of non-antibody-based cancer drugs, for example, oxaliplatin and lenalidomide. Cytokine release syndrome is one of the most frequent and serious adverse effects that occurs after administration of a T cell-engaging immunotherapeutic agent. T cell-engaging immunotherapies include bispecific antibody constructs and chimeric antigen receptor (CAR) T cell therapies, both of which have shown therapeutic efficacy in several hematologic malignancies, including diffuse large B cell lymphoma. Cytokine release syndrome can occur over the course of days or weeks after treatment, or soon after treatment as immediate-onset cytokine release syndrome. Normally, cytokine signaling results in a quick and strong immune response. This response is usually balanced and dissipates when malignant or infected cells have been eliminated. However, in some cases, this positive feedback loop, where activated cells continue to release more cytokines and activate more cells for cytokine release, gets out of control, resulting in a cytokine syndrome that produces excessively high levels of pro-inflammatory cytokines.

Cytokine release syndrome often presents as a combination of fever, hypoxia, hypotension and capillary leak syndrome, with or without organ manifestation. Cytokine release syndrome is caused by a large, rapid release of cytokines into the blood from immune cells, for example, T cells, affected by the immunotherapy.

Cytokines are a large group of proteins, peptides and glycoproteins that are secreted by specific cells of the immune system. Cytokines are signaling molecules that are transiently produced, after cellular activation, to help mediate and regulate immunity, inflammation and hematopoiesis. These molecules act as regulators that modulate the functions of individual cells. Cytokines can act locally, as autocrine, paracrine or endocrine response modifiers, and their actions are exerted via specific cell-surface receptors of their target cells. As used herein, autocrine or autocrine action, means that a cytokine exerts its action by binding to a receptor on the membrane of the same cells that secreted it. Paracrine or paracrine action means that a cytokine binds to a receptor on a target cell in close proximity to a cell that produced the cytokine. Endocrine or endocrine action means that a cytokine travels through circulation and acts on target cells in parts throughout the body.

Elevated levels of cytokines, for example, one or more cytokines selected from the group consisting of IL-1β, IL-2, IL-6, IL-8, MIP1b, MCP1, IL-10, IFN-γ, TGF-β, and TNF-α, are often associated with cytokine release syndrome. Table 5, below, lists the principle cytokines associated with cytokine release syndrome and their effects (Yildizahn and Kaynar, Journal of Oncological Sciences, 4(3): 134-141 (2018)).

TABLE 5

| Cytokine | Source | Target and Effect |
|---|---|---|
| IFN-γ | NK cells, Thl cells and CTLs | Macrophage activation, Thl cell differentiation, B cell |
| | | isotype switching increases MHC expression and |
| | | antigen processing to T cells |
| TNF-α | Macrophages, NK cells and T | Endothelial cell activation (inflammation), microbicidal |
| | cells | activity in neutrophils and macrophages, |
| | | synthesis of acute phase proteins in liver |
| IL1 β | Macrophages, DCs, fibroblasts, | Endothelial cell activation (inflammation, coagulation), |
| | endothelial cells, hepatocytes | synthesis of acute phase proteins in liver |
| IL2 | T cells | proliferation and differentiation of T cells and NK cells |
| | | B cell proliferation and antibody synthesis |
| IL6 | T cells, monocytes, | Augment immune response proliferation of antibody- |
| | macrophages, fibroblasts, and | producing B cells |
| | endothelial cells | Neutrophil production from the bone marrow synthesis |
| | | of acute phase proteins in Liver |
| IL10 | Th2 cells and macrophages | inhibition of the expression of IL-12 in Macrophages |
| | | and DCs |
| IL12 | Macrophages and DCs | Thl cell differentiation |
| | | IFN-γ synthesis in NK cells and T cells increasing |
| | | cytotoxicity |
| IL8 | Macrophages, epithelial cells, | Lymphocyte and neutrophil chemotaxis and induction of |
| | airway myocytes, monocytes, T | phagocytosis (migration exocytosis; release of some |
| | lymphocytes, neutrophils, | mediator such as histamine), respiratory burst, |
| | vascular endothelial cells, | Chemoattractant for endothelial cell, macrophage, mast |
| | dermal fibroblasts, keratinocytes, | cell and keratinocyte promotes angiogenic responses in |
| | hepatocytes | endothelial cell, autocrine growth factor for cancer cells |
| Fractalkine | Monocytes, endothelial cells, | Membrane binding form; leukocytes adhesion |
| | macrophages, DCs, fibroblasts | Soluble form; chemoattractant for monocytes, NK cells |
| | (by stimulation of cytokines such | and T lymphocytes |
| | as TNF-α, IN-γ and IL1-β) | An important receptor and surface marker for NK cells |
| | | and CTL |

### III.C.1.a. Mechanism

Cytokine release syndrome is usually due to on-target effects induced by binding of a bispecific antibody or CAR T cell receptor to its antigen and subsequent activation of bystander immune cells and non-immune cells, such as endothelial cells. Activation of the bystander cells results in the massive release of a range of cytokines. Depending on a number of characteristics of the host, the tumor, and the therapeutic agent the administration of T cell-engaging therapies can set off an inflammatory circuit that overwhelms counter-regulatory homeostatic mechanisms and results in a cytokine syndrome that can have detrimental effects on the subject.

Upon administration of an immunotherapy, activation of T cells or lysis of immune cells induces release of interferon gamma (IFN-γ) or tumor necrosis factor alpha (TNF-α). TNF-α elicits flu-like symptoms similar to IFN-γ with fever, general malaise, and fatigue and is also responsible for watery diarrhea, vascular leakage, cardiomyopathy, lung injury, and the synthesis of acute phase proteins (for example, C-reactive protein). IFN-γ causes fever, chills, headache, dizziness, and fatigue. Secreted IFN-γ induces activation of macrophages, dendritic cells, other immune cells and endothelial cells. The activated macrophages produce excessive amounts of pro-inflammatory cytokines such as, IL-6, TNF-α, and IL-10. Importantly, macrophages and endothelial cells produce large amounts of interleukin 6 (IL-6) which activates T cells and other immune cells leading to a cytokine syndrome.

Interleukin-6 (IL-6) is a pleiotropic cytokine with anti-inflammatory and proinflammatory properties that plays a central role in host defense due to its wide range of immune and hematopoietic activities, as well as its ability to induce the acute phase response. IL-6 appears to be a central mediator of toxicity in cytokine release syndrome. IL-6 signaling, requires binding to cell-associated gp130 (CD130), which is broadly expressed, and the IL-6 receptor (IL-6R) (CD126). IL-6R is expressed on macrophages, neutrophils, hepatocytes, and some T cells and mediates classic signaling, which predominates when IL-6 levels are low. However, when IL-6 levels are elevated, soluble IL-6R can also initiate trans-signaling, which occurs on a much wider array of cells. it is likely that anti-inflammatory properties of IL-6 are mediated via classic signaling, whereas proinflammatory responses occur as a result of trans-signaling. High levels of IL-6, present in the context of cytokine release syndrome, likely initiates a proinflammatory IL-6-mediated signaling cascade.

### III.C.2. Pre-Processing: Generating Cytokine Fold Changes

Cytokine release syndrome prediction system 105 includes a cytokine adjustor 150 that aligns cytokine levels with standardized time points and generates cytokine fold changes. For example, for each of one or more subjects, cytokine adjustor 150 may retrieve a time at which a treatment or pre-treatment was initiated for the subject (e.g., from treatment dosage data store 135). The multiple subjects may include a set of subjects associated with data to be used to train a machine learning model and may also include the particular subject.

For each of the multiple subjects, cytokine adjustor 150 can use the time at which the treatment or pre-treatment was initiated to define the baseline time period. For example, the baseline time period may be defined to end at the time at which the treatment or pre-treatment was initiated (or a predefined time before such initiation, such as a day before the treatment initiation). In some instances, the baseline time period is to have a predefined duration, and cytokine adjustor 150 can identify a beginning of the baseline time period based on the duration and end time for the baseline time period. In some instances, the baseline time period is defined only based on the end time, such that all times preceding the end time are within the baseline time period.

For each of the multiple subjects, cytokine adjustor 150 may retrieve (e.g., from raw cytokine level data store 145) a measurement time associated with each cytokine level stored in association with an identifier for the subject (e.g., from raw cytokine level data store 145). Cytokine adjustor 150 can use the baseline time period and the measurement time(s) associated with the cytokine level(s) to detect which cytokine levels are associated with measurement times within the baseline time period. Cytokine adjustor 150 can characterize each cytokine level associated with a measurement time within the baseline time period as a baseline cytokine level 155.

For each of the set of subjects (associated with data to be used for training) and potential for the particular subject, cytokine adjustor 150 can further retrieve (e.g., from treatment dosage data store 135) a time at which the treatment or a cycle of the treatment concluded. In some instances, a duration of the treatment or a duration of a cycle of the treatment is known (e.g., with a given degree of confidence and a given degree of precision) or has been estimated, such that a time at which the treatment or a cycle of the treatment has concluded or will conclude can be estimated.

For each of the set of subjects and potentially for the particular subject, cytokine adjustor 150 can define an on-treatment time period to begin (for example) a time at which a treatment began or at which a cycle began (e.g., as identified in data retrieved from treatment dosage data store 135). It will be appreciated that a beginning of an on-treatment time period may be different than an end of a baseline time period. In some instances, a treatment specification identifies a time at which treatment administration ended or administration of a cycle of treatment ended, and cytokine adjustor 150 can define an end of the on-treatment time period to conclude at that time. In some instances, a duration of a treatment or of a cycle of treatment is known (e.g., with a given degree of confidence and a given degree of precision), and cytokine adjustor can define the end of the on-treatment time period based on the duration and the beginning of the on-treatment time period.

Cytokine adjustor 150 can use the on-treatment time period and the measurement time(s) associated with the cytokine level(s) to detect which cytokine levels are associated with measurement times within the on-treatment time period. Cytokine adjustor 150 can characterize each cytokine level associated with a measurement time within the on-treatment time period as an on-treatment cytokine level 160. For each of the set of subjects, cytokine adjustor 150 may further characterize each cytokine level that is associated with a measurement time subsequent to the on-treatment time period as a post-treatment cytokine level.

Cytokine adjustor 150 can use one or more baseline cytokine levels 155 and one or more on-treatment cytokine levels 160 to generate at least one cytokine fold change 170. A cytokine fold change 170 may be determined by subtracting a baseline-level term from another term. For a given subject, the baseline-level term can be defined to be or to be based on at least one baseline cytokine level 155, and the other term can be defined to be or to be based on at least one on-treatment cytokine level 160 or at least one other baseline cytokine level 155.

A reference cytokine level can be defined to be a cytokine level that is associated with a measurement time that is within a particular portion of the baseline time period. For example, the reference cytokine level can include a baseline cytokine level 155 measured between 6-8 days before the pretreatment. In some instances, a cytokine fold change 170 is defined for each cytokine level measured for a given subject based on the reference cytokine level and the raw cytokine level.

For each term of the baseline-level term and the other term, the term may be determined using a log function. However, a log of zero is undefined. Thus, a log of a sum of a corresponding cytokine level and a predefined positive value may be calculated instead of calculating a log of the corresponding cytokine level. The predefined value may be, for example, a fraction, 1, 2, etc.

### III.C.3. Machine Learning Model Training

Cytokine release syndrome prediction system 105 includes a model training sub-system 175 that trains one or more machine learning models to predict a cytokine release syndrome risk 180 based on one or more baseline characteristics 115 and a treatment dosage 135. Model training sub-system may also or alternatively predict a cytokine release syndrome risk 180 based on cytokine fold changes 170. Cytokine release syndrome prediction system 105 may further use one or more baseline cytokine levels 155 to predict cytokine release syndrome risk 180. The machine learning model(s) may include (for example) a random forest model, a regression model (e.g., a linear, logistic regression model), a decision tree model, and/or a neural network.

The training data that model training sub-system 175 uses to train the predictive model can be associated with the set of subjects and can include a treatment dosage and an indication as to whether (and when) each subject experienced a cytokine release syndrome and, if so, a grade of the event. Criteria for determining a grade of a cytokine release syndrome can include that as identified in Section III.A.1.a.

Model training sub-system 175 can obtain the cytokine release syndrome information from a cytokine release syndrome (CRS) reports data store 182. CRS reports data store 182 may include multiple CRS report records, each identifying a subject and, for each CRS event, a grade of the cytokine release syndrome, and a time of the cytokine release syndrome. Each CRS report record may be generated based on data or input received from a care provider system 120 associated with the subject (e.g., by virtue of a care provider corresponding to care provider system 120 administering a treatment to the subject, diagnosing the cytokine release syndrome, and/or treating the cytokine release syndrome). Thus, model training sub-system 175 can query CRS reports data store 182 to determine, for each of the set of subjects, whether a cytokine release syndrome (e.g., of at least a threshold grade) was observed.

### III.C.3.a. Training a Decision Tree Model to Transform Baseline Parameters, Dosage and Cytokine-Level Inputs into Predicted Cytokine Release Syndrome Risk

In some instances, model training sub-system 175 can define, for each of the set of subjects, a training data element to include an indication as to whether a cytokine release syndrome (e.g., of at least a threshold grade) was observed, baseline parameters, a treatment dosage, and one or more cytokine fold changes 170 (e.g., corresponding to an on-treatment time period). The training data may be composed of the training data elements that correspond to the set of subjects. The indication as to whether a cytokine release syndrome (e.g., of at least a threshold grade) was observed can be defined as a label for the training data element.

In some instances, model training sub-system 175 can train a model to learn a set of model parameters that facilitate transforming the baseline parameters (as single baseline characteristics 115 or as a baseline cytokine release syndrome risk score 184) into a cytokine release syndrome risk 180. In some instances, model training sub-system can train a model to learn a set of model parameters that facilitate transforming baseline parameters, treatment dosage 135, and/or cytokine fold change(s) 170 into cytokine release syndrome risk 180. The learning model can include (for example) a decision tree model 183 and the model parameters can include a set of decision tree thresholds.

The decision tree thresholds of the predictive model can include a dosage threshold, baseline cytokine release syndrome risk score (CRSRS) threshold, and one or more cytokine-level thresholds. Thus, decision tree model 183 can determine whether the treatment dosage exceeds a dosage threshold, whether baseline cytokine release syndrome risk score exceeds a CRSRS threshold, and/or whether a cytokine fold change 170 exceeds each of one or more cytokine-level thresholds. For example, a different (e.g., lower) cytokine-level threshold may be used when the dosage exceeds the dosage threshold as compared to when the dosage does not exceed the dosage threshold. In some instances, each training data element includes multiple cytokine fold changes 170. Decision tree model 183 may be configured to identify each “on-treatment” cytokine fold change 170 that corresponds to an on-treatment time period and to use the maximum on-treatment cytokine fold change for the threshold comparison.

### III.C.3.b. Training a Feature Selection Model and/or Risk-Score Generation Model to Transform Baseline-Characteristic Inputs into a Risk Score

In some instances, at least some of set of baseline characteristics (e.g., retrieved from baseline characteristics data store 115) can be used (e.g., in conjunction with the treatment dosage and cytokine fold change(s) 170) to predict cytokine release syndrome risk 180. For example, a risk-score generation model 184 can transform the at least some of the set of baseline characteristics into a cytokine release syndrome risk score, which may then be used as single predictor by decision tree model 183 to predict cytokine syndrome risk 180.

Model training sub-system 175 can perform a feature selection to identify which baseline characteristics are to be used by risk-score generation model 184 to predict cytokine release syndrome risk 180. In some instances, the features are selected by using a feature selection model 185, which may be configured to perform, for each baseline characteristic, a univariate analysis. The univariate analysis may output a significance value that may indicate whether there is a significant relationship between the characteristic and an indication as to whether a cytokine release syndrome occurred (e.g., that was of at least a threshold grade, such as an event of at least Grade 1 severity or an event of at least Grade 2 severity). An initial subset of the baseline characteristics can be defined to be those baseline characteristics with a p-value below a predefined threshold (e.g., below 0. 1 or below 0. 3). This subset can be refined further by 185 by applying multivariate techniques, such as floating forward/backward multiple regression or random forest analyses.

In some instances, feature selection model 185 may perform k-fold cross validation. The cross validation may be performed multiple times, where each cross-validation performance is associated with a subset of the baseline characteristics. For each cross-validation performance and at each fold, training data can be divided into a training portion and a testing portion used to assess performance of the model. The training data used for feature selection can include, for each of a set of subjects, the set of baseline statistics and an indication as to whether a cytokine release syndrome (e.g., of at least a threshold grade) was observed.

Stratification factors can be defined to include disease histology and treatment dosage. Thus, the training and testing data sets used for feature selection can be defined to include approximately the same distribution of disease histology in the training data set as in the testing data set, and approximately the same distribution of treatment dosages in the training data sets as in the testing data set. The training data set can be used to train feature selection model, and the testing data set can be used to determine a performance metric. A performance statistic can be generated for each baseline-characteristic subset based on the performance metrics across the corresponding folds. A reduced feature set can be defined to be the subset associated with the highest performance and stability statistics.

Model training sub-system 175 can then train risk-score generation model 184 to learn a set of model parameters (e.g., a set of weights) to transform values for the reduced feature set into a cytokine release syndrome risk score. Risk-score generation model 184 may include a regression model or weighting sum of baseline parameter values. In some instances, learning set of model parameters can include learning a parameter value for each baseline characteristic represented in the reduced feature set (e.g., a corresponding subset of the set of baseline characteristics). For example, the parameters may include a weight for each baseline characteristic represented in the reduced feature set.

The model parameters can be learned by (for example) fitting one or more functions to the training data. For example, model training sub-system may learn a weight for each baseline characteristic represented in the reduced feature set, where the weights can be identified as those that maximized classification accuracy and stability in the training data. Each weight can be primarily derived from log (Cytokine Release Syndrome Odds Ratio) in the dose-adjusted logistic regression model that models Cytokine Release Syndrome Odds Ratio in terms of the involved parameter value and drug dose (exposure). The weight can be further tuned by including information about the variable's stability as in the random forest and floating feature selection experiment.

Additionally or alternatively, model training sub-system 175 can learn parameters of risk-score generation model 184 by using a loss function by iteratively using the machine learning model and a loss function. For example, the machine learning model can generate one or more predicted outputs using parameter values for the reduced feature set (e.g., predicting whether a cytokine release syndrome occurred), compare the predicted output(s) to label(s) in the training data, calculate a loss based on the comparison and on a loss function, and adjust parameters of risk-score generation model 184 based on the loss. This process may be repeated across multiple training cycles. After a loss or moving average of losses falls below a predefined loss threshold and/or after a number of training cycles crosses a predefined training-cycle threshold, model training sub-system 175 can fix the set of parameters.

Model training sub-system 175 may be configured to train risk-score generation model 184, to tune a trained version of risk-score generation model 184, or to configure a post-processing algorithm to transform—not only the selected subset of model parameters—but also one or more treatment specifications (e.g., a treatment dosage) into a predicted risk of a given subject experiencing a cytokine release syndrome (e.g., of at least a threshold grade). For example, parameters of risk-score generation model 184 may be learned by feeding input data that includes values for the subset of baseline characteristics into risk-score generation model 184, determining whether and/or a degree to which a cytokine release syndrome risk score output by the model accurately predicted whether a cytokine release syndrome (e.g., of at least a threshold grade) was observed, and calculating a loss based on whether and/or a degree to which the cytokine release syndrome risk score output was accurate. The set of parameters may be used irrespective of a treatment dosage administered to a subject, or a different set of parameters may be learned for each of multiple treatment dosages. As another example, risk-score generation model 184 may first be trained to learn a set of parameters for predicting cytokine release syndrome risk scores based on baseline characteristics. Risk-score generation model 184 may then use the set of parameters, baseline characteristics, and a treatment dosage to determine a cytokine release syndrome risk for a given subject.

### III.C.3.c. Training a Decision Tree Model to Transform Risk-Score and Cytokine-Level Inputs into Predicted Cytokine Release Syndrome Risk

In some instances, decision tree model 183 is configured to receive one or more cytokine levels and a risk score as input. The risk score may be a cytokine release syndrome risk score (determined using baseline characteristics in 180). Decision tree model 183 may further receive the treatment dosage as an additional input variable.

Model training sub-system 175 can train decision tree model 183 to learn a set of thresholds, which can include a risk-score threshold and one or more cytokine-level thresholds. Thus, decision tree model 183 can determine whether the risk score 180 exceeds a risk-score threshold and/or whether a cytokine fold change 170 exceeds each of one or more cytokine-level thresholds. In some instances (e.g., when decision tree model 183 receives a cytokine release syndrome risk score 180 as input, the set of thresholds can further include a dosage threshold, and decision tree model 183 can determine whether the dosage exceeds the dosage threshold.

The one or more cytokine-level thresholds can include multiple thresholds. For example, a different (e.g., lower) cytokine-level threshold may be used when a cytokine release syndrome risk score exceeds the risk-score threshold (as compared to when the risk score does not exceed the risk-score threshold) and/or when a dosage exceeds the dosage threshold (as compared to when the dosage does not exceed the dosage threshold). In some instances, each training data element includes multiple cytokine fold changes 170. Decision tree model 183 may be configured to identify each “on-treatment” cytokine fold change 170 that corresponds to an on-treatment time period and to use the maximum on-treatment cytokine fold change for the threshold comparison.

### III.C.4. Predicting Cytokine Release Syndrome Risk

Cytokine release syndrome prediction system 105 includes a CRS risk detector 190 that uses one or more trained machine-learning models to transform a subject-specific input data set corresponding to the particular subject into a particular cytokine release syndrome risk 180. The subject-specific data set can include one or more baseline characteristics (e.g., that characterize a tumor burden, tumor spread, presence or quantity of malignant cells within peripheral blood, presence or quantity of malignant cells with bone marrow, a demographic attribute, age, baseline LDH level, baseline WBC level, and/or a co-morbidity). The subject-specific data set can further include one or more cytokine fold changes 170 associated with the particular subject and a treatment dosage associated with a particular subject (e.g., indicating a dosage of the treatment that has been administered to the subject, a dosage of the treatment that has been prescribed for the subject, or a dosage of the treatment that is being considered for the subject) Thus, for example, a subject-specific data set may include: (1) one or more baseline characteristics; (2) one or more baseline characteristics and a treatment dosage; or (3) one or more baseline characteristics, a treatment dosage, and one or more cytokine fold changes 170.

CRS risk detector 190 may combine the one or more cytokine fold changes 170 associated with the particular subject and one or more other subject-specific values to decision tree model 183 to generate cytokine release syndrome risk 180 for the particular subject (representing a predicted risk of the subject experiencing a cytokine release syndrome subsequent to receiving the dosage of the treatment). The one or more other subject-specific values can include the treatment associated with the particular subject and/or the risk score. The predicted risk may (for example) include a categorical value (e.g., representing very low risk, low risk, moderate risk, high risk, or very high risk) or a binary value (e.g., representing high risk or not high risk).

CRS risk detector 190 can access an in-patient monitoring condition 193 and evaluate the condition using cytokine release syndrome risk 180 generated for the particular subject. In-patient monitoring condition 193 can be configured to be satisfied when a cytokine release syndrome risk is a particular value (e.g., high risk) or is above a particular threshold (e.g., moderate or higher risk).

CRS risk detector 190 may select or may generate an output to be availed to user device 110 by cytokine release syndrome prediction system 105 based on the condition evaluation. For example, an output of “Consider in-patient monitoring” or “In-patient monitoring is recommended” may be selected when in-patient monitoring condition 193 is satisfied, and an output of “Consider out-patient monitoring” or “Out-patient monitoring recommended” may be selected when in-patient monitoring condition is satisfied. The output may further include (for example) one or more cytokine fold changes (e.g., used to generate cytokine release syndrome risk 180), one or more numeric risk scores, one or more raw cytokine levels, one or more baseline characteristics, and/or a dosage that were associated with the particular subject.

User device 110 may present the output to a user. The user (or another entity) may determine whether to accept the recommendation and may then facilitate in- or out-patient monitoring accordingly.

### III.D. Exemplary In- or Out-Patient Monitoring

If the particular subject is monitored as an out-patient, the particular subject may be advised (e.g., by a care provider) to monitor for any of one, more, or all of the symptoms identified in Section III.D.1. and to alert a care provider or to go to a medical facility if any of the symptoms occur.

If the particular subject is monitored as an in-patient, a care provider (e.g., a physician and/or nurse) may monitor for any of one, more, or all of the symptoms identified in Section III.D.1, and the particular subject may also be asked to monitor for any such symptom. Further, if the particular subject is monitored as an in-patient, one or more laboratory tests may be periodically performed (e.g., to detect cytokine levels) to facilitate quickly detecting any cytokine release syndrome.

III.D.1. Symptoms

Cytokine release syndrome symptoms can range from mild, flu-like symptoms to severe life-threatening symptoms. Mild symptoms of cytokine release syndrome include fever, fatigue, headache, rash, arthralgia, and myalgia. More severe cases are characterized by hypotension, as well as high fever, and can progress to an uncontrolled systemic inflammatory response with vasopressor-requiring circulatory shock, vascular leakage, disseminated intravascular coagulation, and multi-organ system failure. Respiratory symptoms are common in subjects with cytokine release syndrome. Mild cases may display cough and tachypnea but can progress to acute respiratory distress syndrome (ARDS) with dyspnea, hypoxemia, and bilateral opacities on chest X-ray.

Timing of symptom onset and cytokine release syndrome severity depends on the immunotherapeutic and the magnitude of immune cell activation. Cytokine release syndrome following rituximab for CD20+ malignancies typically occur within minutes to hours, and subjects with >50×109/L circulating lymphocytes have increased rates of cytokine release syndrome symptoms (Winkler et al. Blood, 94(7): 2217-2224 (1999)). In contrast, symptom onset typically occurs days (for CAR T cell therapy) to weeks (for cytotoxic T cell (CTL) therapy) after the T-cell infusion, coinciding with maximal in vivo T-cell expansion (Lee et al. Blood, 124(2): 188-195 (2014)).

Symptoms and severity associated with cytokine release syndrome vary greatly, and management can be complicated by concurrent conditions in these subjects. Fever is a hallmark of cytokine release syndrome, and many features of cytokine release syndrome mimic infection. Since it is not uncommon for subjects to experience temperatures exceeding 40.0° C., infection is considered as an alternative explanation in all subjects presenting with cytokine release syndrome symptoms.

Potentially life-threatening complications of cytokine release syndrome include cardiac dysfunction, adult respiratory distress syndrome, neurologic toxicity, renal and/or hepatic failure, and disseminated intravascular coagulation. Of particular concern is acute cardiac toxicity in the setting of cytokine release syndrome, which resembles cardiomyopathy associated with sepsis and stress cardiomyopathy. Neurologic symptoms occurring in the context of cytokine release syndromes are varied. Neurologic symptoms may occur together with other symptoms of cytokine release syndrome or may arise when the other symptoms of cytokine release syndrome are resolving.

Cytokine release syndrome may also be associated with findings of macrophage activation syndrome/hemophagocytoic lymphohistiocytosis (HLH), and the physiology of the syndromes may have some overlap. In subjects with cytokine release syndrome who develop a HLH/MAS-like syndrome, additional cytokines such as IL-18, IL-8, IP-10, MCP1, MIG, and MIP1β are also elevated. These cytokines also have been reported to be elevated in classical HLH and MAS. Some subjects may have genetic variants that predispose them to developing HLH/MAS. In addition, IL-6 may also promote the development of HLH/MAS, in the setting of cytokine release syndrome, by inducing dysfunctional cytotoxic activity in T and NK cells, which is a hallmark of HLH and MAS.

Tumor lysis syndrome may also occur coincident with cytokine release syndrome, because massive immune cell activation and expansion correlates with antitumor efficacy.

### III.D.2. Diagnosis

If the particular subject is being monitored as an in-patient, a care provider may determine whether to diagnose the particular subject with having a cytokine release syndrome (e.g., after one or more symptoms were observed). Similarly, if the particular subject is being monitored as an out-patient but subsequently arrives at a medical facility (e.g., after observing one or more cytokine release syndrome symptoms), the care provider may determine whether the particular subject with having a cytokine release syndrome.

Cytokine release syndrome is diagnosed in the context of the underlying medical condition of the particular subject. This underlying problem might already be known, or it may require its own diagnosis. In the context of treatment of non-Hodgkin's lymphoma, factors that may influence treatment selection frequently include the particular subject's non-Hodgkin's lymphoma subtype, cycle and the type of therapy that has been administered to the subject. Medical history and physical exam provide diagnostic starting points.

The care provider can examine the subject for signs that might indicate occurrence cytokine release syndrome, because cytokine release syndrome can affect many different systems of the body. As indicated above, abnormally low blood pressure, fever, and hypoxia may be indicative of a cytokine release syndrome.

Laboratory tests may be performed to identify abnormalities. An increased levels of one or more cytokine, decreased number of immune cells; elevation in a marker of kidney or liver damage; elevations in an inflammatory marker, like C-reactive protein; abnormality in a marker of blood clotting; and elevated ferritin are all consistent with occurrence of a cytokine release syndrome.

Medical imaging can be performed. For example, a chest X-ray or CT scan could identify lung involvement from cytokine release syndrome.

Based on the results of the physical examination, laboratory tests, medical imaging, etc., the care provider can determine whether the subject has cytokine release syndrome and, if so, assign a grade of the cytokine release syndrome to the subject. Grading or staging of cytokine release syndrome guides treatment options. Prior to determining that a cytokine release syndrome occurred, a care provider may exclude other potential medical conditions that may be consistent with the results of the physical examination, laboratory tests, medical imaging, etc. For example, the care provider may rule out the particular subject as suffering from any of: infection, neutropenic sepsis, tumor lysis syndrome, or adrenal insufficiency, as anti-cytokine therapy administered under these conditions and without clear evidence of cytokine release syndrome may be detrimental.

The National Cancer Institute Common Terminology Criteria for Adverse Events (CTCAE v4.0) contains the following grading system that was designed for cytokine release syndrome associated with antibody therapeutics. Table 6 shows characteristic symptoms and treatment recommendations for each grade of cytokine release syndrome.

TABLE 6

| | Symptoms | Treatment |
|---|---|---|
| Grade 1 | Mild constitutional symptoms such | Not require interruption of therapy |
| | as fever, nausea, fatigue, headache, | Symptomatic treatment ± empiric treatment |
| | myalgia, malaise | of concurrent bacterial infections |
| Grade 2 | Symptoms require moderate | interruption of therapy is required, rapid |
| | intervention | response to symptomatic treatment |
| | Hypoxia with oxygen | immunosuppressive treatment is optional |
| | requirement less than 40% | according to the comorbidities or age |
| | Hypotension responds to | |
| | fluids or low dose vasopressor | |
| | Grade 2 organ toxicity | |
| Grade 3 | Symptoms require aggressive | Prolonged duration of symptoms despite |
| | intervention | symptomatic treatment and interruption of |
| | Hypoxia with oxygen | therapy. |
| | requirement more than 40% | Monitorization in ICU aggressive |
| | Hypotension not responsive | intervention with immunosuppressive |
| | to low dose vasopressor (requires | treatment (tocilizumab ± corticosteroids) is |
| | high dose or multiple vasopressors) | required. |
| | Grade 3 organ toxicity -such | |
| | as coagulopathy, renal dysfunction, | |
| | cardiac dysfunction- or grade 4 | |
| | transaminitis | |
| Grade 4 | Life-threatening symptoms and | Ventilator support and vasopressors are |
| | toxic condition | required |
| | Grade 4 organ toxicity | Rapid intervention with immunosuppressive |
| | (excluding transaminitis) | treatment (tocilizumab ± corticosteroids) |
| Grade 5 | Death | |

### III.D.3. Treatment of Cytokine Release Syndrome

If it is detected that the particular subject is experiencing a cytokine release syndrome, a care provider may administer or provide a treatment identified in this section. Management of cytokine release syndrome can follow a grade- and risk-adapted strategy for monitoring and therapy (Shimabukuro-Vornhagen et al. J. Immunother. Cancer 6: 56 (2018)).

A low grade cytokine release syndrome can be treated symptomatically with antihistamines, antipyretics, analgesics, and fluids. Additional diagnostic testing can frequently be performed to rule out differential diagnoses. If an infection cannot be ruled out with certainty, an empiric antibiotic therapy can be considered. Furthermore, if the particular subject presents with early signs of a cytokine release syndrome, a frequency of actively monitoring the subject (via in-patient monitoring) for signs of further deterioration may be increased.

A severe cytokine release syndrome represents a life-threatening situation that requires prompt and aggressive treatment. Thus, if the particular subject experiences a severe cytokine release syndrome, a treatment for the cytokine release syndrome may be promptly administered. The cytokine release syndrome treatment may include anti-cytokine therapy, e.g., tocilizumab, with or without a corticosteroid (e.g., for a grade 3 or higher cytokine release syndrome or for grade 2 in high-risk subjects). In some instances, depending on the type of immunotherapy, a corticosteroid, without anti-cytokine therapy, can be administered at grade 1, rather than waiting until the subject has grade 2 cytokine release syndrome or higher, to reduce the rate of immunotherapy-related cytokine release syndrome and neurologic events. See, for example, Liu et al. Blood Cancer J. 10(2): 15 (2020). As another example, blinatumomab (an immunotherapeutic for treatment of acute lymphoblastic leukemia) may be administered to the particular subject in response to detecting a severe cytokine release syndrome.

Since IL-6 is elevated in the serum of subjects with cytokine release syndrome following immunotherapy, for example, CAR T cell therapy or bispecific T-cell engager therapy, tocilizumab (an anti-IL-6 therapy) can be administered to the particular subject if diagnosed as having a severe cytokine release syndrome. IL-6 can be a suitable target because IL-6 is not critical for T cell function, but, as set forth above, drives many symptoms of cytokine release syndrome. By binding to membrane-bound as well as soluble IL-6 receptor, tocilizumab can interfere with both classical and trans-signaling pathways. Studies confirmed that administration of monoclonal antibodies against IL-6 (siltuximab) and its receptor (tocilizumab) led to rapid resolution of cytokine release syndrome symptoms (Shimabukuro-Vornhagen (2018)). In an early stage clinical trial, tocilizumab demonstrated a 69% response rate in subjects with severe or life-threatening cytokine release syndrome. As a consequence, tocilizumab is often used for the initial treatment of severe cytokine release syndrome in subjects receiving CAR T cells.

In August 2017, the FDA approved tocilizumab for the treatment of cytokine release syndrome in subjects 2 years of age or older. The approved dosage of tocilizumab for cytokine release syndrome is 12 mg/kg for subjects less than 30 kg weight and 8 mg/kg for subjects at or above 30 kg weight. Fever and hypotension generally ameliorates within a few hours in subjects responsive to tocilizumab. However, in some subjects it may be necessary to continue supportive treatment for several days. Although tocilizumab has a very long half-life (11-14 days), the common approach is to repeat the dose, with or without corticosteriods, if enough clinical improvement is not achieved within 48 h. If the subject still does not improve with persisting high IL-6 levels, a high dose of tocilizumab may be considered. Subjects that develop grade 3 or 4 cytokine release syndrome are typically administered treatment (e.g., tocilizumab, with or without corticosteriods) nearly immediately. It is important to note that, after administration of tocilizumab, C-reactive protein can no longer be used as an indicator of cytokine release syndrome severity, as blockade of IL-6 signaling results in a rapid decrease of C-reactive protein.

There are several other IL-6-targeting monoclonal antibodies in late stage clinical development for treatment of cytokine release syndrome. Siltuximab, is a chimeric, IGκ monoclonal antibody that binds human IL-6 and prevents it from interacting with both the membrane-bound and soluble form of the IL-6 receptor. Clazakizumab is another monoclonal antibody targeting IL-6.

Monoclonal antibodies that target IL-6 directly, thereby eliminating it from the circulation, may be used to treat the particular subject, if the subject is experiencing severe cytokine release syndrome and concurrent neurotoxicity, since tocilizumab does not cross the blood brain barrier and therefore fails to inhibit IL-6 signaling in the CNS. Corticosteroids may be used to treat the particular subject when HLHI/MAS is developed as part of the cytokine release syndrome. If corticosteroids are used in subjects receiving T cell-engaging immunotherapy, the duration of treatment may be kept as short as possible to minimize any potential detrimental effects on the effectiveness of the immunotherapy.

In cases where neither tocilizumab nor glucocorticoids are effective, blockade of TNF-α signaling can be used. However, there are cases of severe cytokine release syndrome that are unresponsive to tocilizumab, etanercept (anti-TNF antibody), and glucocorticoids. In those cases, other immunosuppressants, such as the IL-6 monoclonal antibody siltuximab, T cell-depleting antibody therapies, such as alemtuzumab and ATG, IL-1R-based inhibitors (anakinra), or cyclophosphamide, may be administered or provided.

Other experimental therapies for cytokine release syndrome include ibrutinib. Also, cytokine adsorption, may be effective in treating cytokine release syndrome. An advantage of extracorporeal cytokine adsorption over other therapeutic approaches is that it does not selectively block a specific receptor or signal transduction cascade. Instead, this method reduces particularly elevated concentrations of various inflammatory mediators, for example, cytokines with pro- and anti-inflammatory functions, such as IL-6, TNF-α, and interferons. In these methods, blood is taken from a subject's circulation, and cytokines are removed from the blood, before it is returned to the circulation.

### III.E. Exemplary Alternative Embodiments

It will be appreciated that various alternative embodiments to those described above or depicted in FIG. 1 are contemplated with respect to network 100. For example, instead of or in addition to using cytokine release syndrome risk 180 to evaluate in-patient monitoring condition, CRS risk detector 190 uses cytokine release syndrome risk 180 to assign the particular subject to a cohort in a clinical study. The cohort assignment may be based on an algorithm or technique that optimizes or prioritizes defining cohort assignments such that there is high overlap between cohorts in terms of cytokine release syndrome risks. Thus, the cohort assignment may be generated based on cohort assignments and cytokine release syndrome risks 180 associated with at least one other subject. An output from cytokine release syndrome prediction system 105 can identify the cohort assignment.

As another example, instead of or in addition to using cytokine release syndrome risk 180 to evaluate in-patient monitoring condition 193, CRS risk detector 190 uses cytokine release syndrome risk 180 to determine whether a particular eligibility criterion for a clinical study is satisfied. The particular eligibility criterion may require subjects to have a particular cytokine release syndrome risk or to have a cytokine release syndrome risk that is at least a threshold value to be enrolled in the clinical study. Thus, the criterion may be evaluated using the particular subject's cytokine release syndrome risk 180 to determine a criterion-specific result. If the criterion is not satisfied, an output may indicate that the particular subject is ineligible for the clinical study. If the criterion is satisfied, it may be determined whether each remaining criteria is satisfied, and an output may indicate whether the subject is eligible for the study.

As yet another example, instead of or in addition to using cytokine release syndrome risk 180 to evaluate in-patient monitoring condition 193, CRS risk detector 190 uses cytokine release syndrome risk 180 to determine whether to recommend, provide and/or administer one or more agents to reduce a likelihood of a cytokine release syndrome occurring. The one or more agents can include (for example) a steroid agent (e.g., a corticosteroid or methylprednisolone) or a cytokine-directed treatment (IL-6 receptor inhibitor, such as tocilizumab).

### IV. Exemplary Processes for Stratifying Subjects for Predicting Cytokine Release Syndrome Risks

### IV.A. Exemplary Process for Predicting Risk of Cytokine Release Syndrome

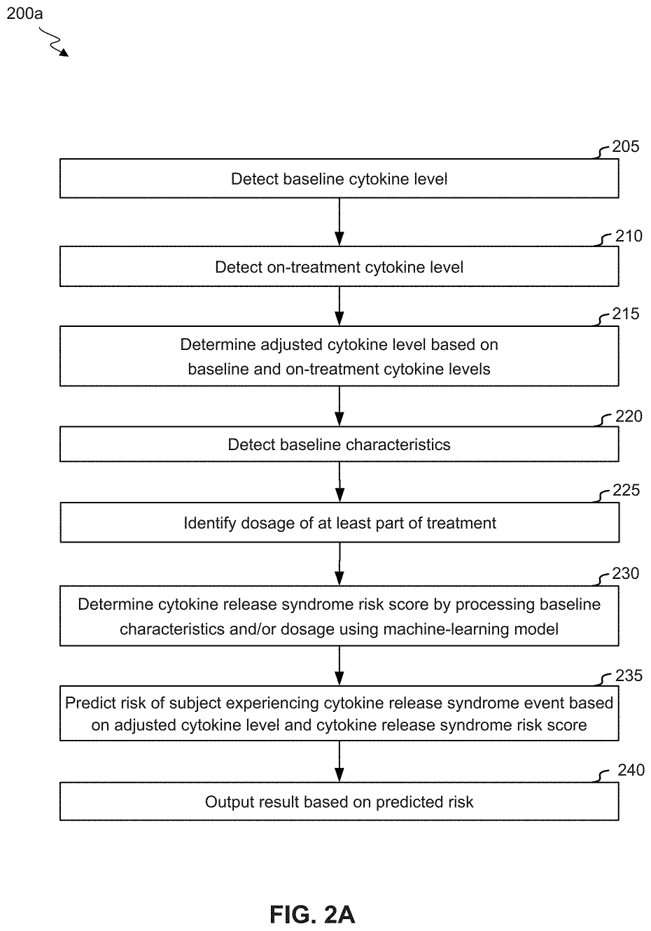

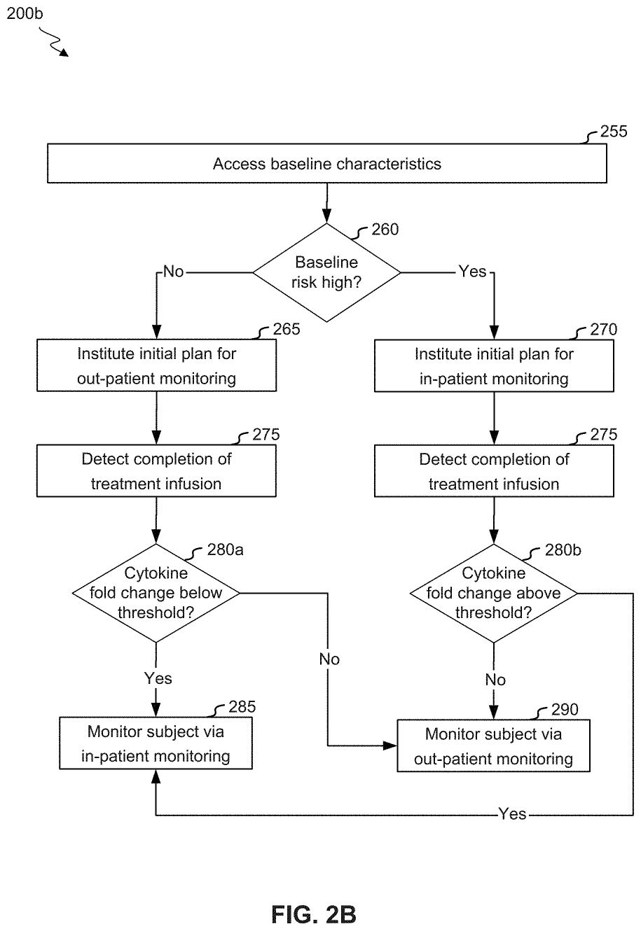

FIG. 2A illustrates a flowchart of a process 200a for predicting a risk that a subject will experience a cytokine release syndrome. Process 200a begins at block 205, where cytokine adjustor 150 detects a baseline cytokine level 155. Detecting baseline cytokine level 155 can include processing one or more cytokine-level data records associated with the subject (e.g., from raw cytokine level data store 145) or inputs associated with the subject to extract a cytokine level associated with a timestamp within a baseline time period.

At block 210, cytokine adjustor 150 detects an on-treatment cytokine level 160. Detecting on-treatment cytokine level 160 can include processing the one or more cytokine-level data records associated with the subject (e.g., from raw cytokine level data store 145) or inputs associated with the subject to extract a cytokine level associated with a timestamp within an on-treatment time period.

At block 215, cytokine adjustor 150 determines cytokine fold change 170 based on the baseline cytokine level and the on-treatment cytokine level. For example, cytokine fold change 170 can be defined to be the on-treatment cytokine level minus the baseline cytokine level. As another example, cytokine fold change 170 can be defined to be a log of the on-treatment cytokine level plus a constant minus a log of the baseline cytokine level plus a constant.

At block 220, CRS risk detector 190 detects one or more baseline characteristics. Detecting the baseline characteristic(s) can include processing one or more baseline-characteristic data records associated with the subject (e.g., from baseline-characteristics data store 115) or inputs associated with the subject to extract the baseline characteristics. In some instances, one or more timestamps associated with one or more data records and/or with one or more inputs are detected, and block 220 includes determining which of the one or more timestamps are within a baseline time period and then extracting information from one or more corresponding data records and/or inputs.

At block 225, CRS risk detector 190 identifies a dosage of at least part of a treatment is identified. The dosage may be identified by (for example) querying treatment dosage data store 135 with an identifier of the subject or by detecting the dosage within input received from user device 110. The dosage may include a dosage of an active ingredient or the treatment. The dosage may include (for example) a dosage within a cycle of a multi-cycle treatment or a cumulative dosage. The dosage may include a dosage (e.g., of an active ingredient or entire treatment) that was already administered to the subject, that is being administered to the subject, that has been prescribed for the subject, or that is being considered as a treatment option for the subject.

At block 230, CRS risk detector 190 determines a cytokine release syndrome risk score by processing the baseline characteristics and optionally the dosage using a machine-learning model. The cytokine release syndrome risk score may represent an interim prediction of a risk of the subject experiencing a cytokine release syndrome (e.g., of at least a threshold grade and/or within a predefined time window from a treatment initiation). The cytokine release syndrome risk score may be determined using risk-score generation model 184. The cytokine release syndrome risk score may be determined by (for example) retrieving one or more learned parameters for risk-score generation model 184 (e.g., a parameter associated with each of two or more characteristics) and generating the risk score using the parameter(s) and the baseline characteristics and optionally the dosage.

In some instances, before the risk score is generated, CRS risk detector 190 uses feature selection model 185 or a result produced by feature selection model 185 to detect a subset of the baseline characteristics that are to be used to determine the risk score. Block 230 may then selectively use the subset of baseline characteristics (potentially along with the dosage) to determine the risk score.

At block 235, CRS risk detector 190 predicts a risk of the subject experiencing a cytokine release syndrome (e.g., of at least a threshold grade and/or within a predefined time period) based on CRSRS and (potentially) dose and the cytokine fold change. The predicted risk of the subject experiencing a cytokine release syndrome may be cytokine release syndrome risk 180 and may be determined using decision tree model 183.

At block 240, cytokine release syndrome prediction system 105 outputs a result based on the predicted risk. The result may be output to a user device 110 that initiated process 200s and/or a care-provider system 120 associated with the subject. The result may identify the predicted risk. The result may further or alternatively identify an action (e.g., to be performed, recommended, or presented for consideration) identified in response to evaluating a condition (e.g., in-patient monitoring condition 193) based on the predicted risk. For example, the result may indicate that the subject is to have or is to be considered for in-patient monitoring for a predefined monitoring period following the treatment. As another example, the result may indicate that the subject is to have or is to be considered for out-patient monitoring for a predefined monitoring period following the treatment. The result may be output via (for example) a transmission or presentation.

It will be appreciated that variations of process 200 are contemplated. For example, one, more, or all of blocks 205, 210, 215, and 225 may be omitted from process 200. As one illustration, each of blocks 205, 210, 215, and 225 are omitted from process 200, and the cytokine release syndrome risk score generated at block 230 is based on (e.g., and/or based only on) one or more baseline characteristics.

### IV.B. Exemplary Process for Selecting In- or Out-Patient Monitoring Based on Risk Prediction

FIG. 2B shows a process 200b for using a predicted risk to determine whether to recommend in- or out-patient monitoring for cytokine release syndromes in a subject. Process 200b begins by accessing baseline characteristics 255, which may include some or all of the baseline characteristics detected at block 220. The baseline characteristic(s) may be used to determine (e.g., by risk-score generation model 184) a baseline risk (e.g., a numeric risk score or categorical score), which may also or alternatively depend on a treatment dosage. One or more baseline cytokine level can also be determined (e.g., by processing a sample collected during a baseline time period).

At block 260, decision-tree model 183 can determine if the baseline risk is high. For example, decision-tree model 183 may compare the risk to a threshold. If the risk is determined to be low, process 200b proceeds to block 265, where an initial plan can be instituted for out-patient monitoring. For example, the subject may be told that it is likely that he or she will be discharged or can leave a medical facility upon completion of the treatment. Meanwhile, if the risk is determined to be high, process 200b proceeds to block 270, where an initial plan can be instituted for in-patient monitoring. For example, the subject may be told that it is unlikely that he or she will be discharged or can leave a medical facility upon completion of the treatment, admission information may be requested from the subject, and/or data can be instituted that reserves a space or a room for the subject for a period of time after the treatment.

At block 275, infusion of the treatment is completed. At this time and/or while the treatment was being infused, one or more on-treatment samples may have been collected from the subject, and one or more on-treatment levels of one or more cytokines within the sample(s) can be measured. One or more on-treatment cytokine levels and one or more baseline levels can be used to determine a cytokine fold change.

When the subject had preliminarily been identified as low risk (at block 265), process 200b proceeds from block 275 to block 280a. At block 280a, it is determined whether the cytokine fold change is below a cytokine-level threshold. In some instances, the cytokine-level threshold is selected based on the prior determination (at block 260) that the subject had been assigned a low-risk classification. If it is determined that the cytokine fold change is below the cytokine-level threshold, process 200b proceeds to block 285 where the subject is monitored via out-patient monitoring. Otherwise, process 200b proceeds to block 290 where the subject is monitored via in-patient monitoring.

When the subject had preliminarily been identified as high risk (at block 265), process 200b proceeds from block 275 to block 280b. At block 280b, it is determined whether the cytokine fold change is above a cytokine-level threshold. In some instances, the cytokine-level threshold is selected based on the prior determination (at block 260) that the subject had been assigned a low-risk classification. Thus, it is possible that the cytokine-level threshold considered at block 280a is different than the cytokine-level threshold considered at block 280b. If it is determined that the cytokine fold change is above the cytokine-level threshold, process 200b proceeds to block 290 where the subject is monitored via in-patient monitoring. Otherwise, process 200b proceeds to block 285 where the subject is monitored via out-patient monitoring.

It will be appreciated that in- and/or out-patient monitoring (at blocks 290 or 285) may indicate that the subject is to actually receive that type of monitoring, a care provider is to recommend in- (or alternatively out-patient monitoring), instructions are to be provided to the subject to prepare for that type of monitoring, and/or a recommendation is to be provided to the subject for the type of monitoring.

### IV. Examples

### IV.A. Example 1: Exemplary Training and Use of Multi-Variate Model to Predict Occurrence of Cytokine Release Syndromes

Clinical data and laboratory values were used to train multiple models (a risk-score generation model and a decision-tree model) to predict the incidence and/or severity of cytokine release syndrome following an administration of a CD 3-engaging bispecific cancer immunotherapy (glofitamab). The degree to which various variables were predictive of the incidence and/or severity of cytokine release syndrome was further characterized. Further yet, the trained models were used to determine the extent to which a subject subset could be identified with a low (<10%) risk of Grade 2+ cytokine release syndrome based on baseline observation and laboratory values.

### IV.A.1. Training/Validation Data

Data used to train and validate the machine learning models were from clinical study NP30179, which was a Phase 1, multicenter, dose-escalation study. One intervention in the study included administering 1000 mg obinutuzumab via IV infusion on Day 1 and administering glofitamab (at a schedule-specified dosage) at one or more subsequent days. Data corresponding to this intervention were analyzed. The study evaluated the efficacy, safety, tolerability, and pharmacokinetics in subjects with relapsed/refractory B-cell non-Hodgkin's lymphoma.

The cohorts assessed in this Example include:

Three fixed dose cohorts:
MQ2W (a monotherapy regimen following a Day-1 pre-treatment): 1000 mg obinutumzumab administered on Day 1, and one of multiple defined dosages of glofitamab administered on each of Days 8, 22, and 36 (where the same dosage was administered on each of Days 8, 22, and 36; and where the dosage was between 0.6 mg and 25 mg);
MQ3W (a monotherapy regimen following a Day-1 pre-treatment): 1000 mg obinutumzumab administered on Day 1, and one of multiple defined dosages of glofitamab administered on each of Days 8, 22, and 43 (where the same dosage was administered on each of Days 8, 22, and 36; and where the dosage was between 0.6 mg and 16 mg);
CQ3W (a combination therapy regimen following a Day-1 pre-treatment)): 1000 mg obinutumzumab administered on Day 0, one of multiple defined dosages of glofitamab and administered on each of Days 8, 22, and 43 (where the same glofitamab dosage was administered on each of Days 22, and 36; and where the dosage was between 0.6 mg and 16 mg); and 1000 mg obinutumzumab was administered on Days 22 and 43;
   One split dose cohort:
10/16 Q3W: 1000 mg obinutumzumab administered on Day 1, 10 mg glofitamab administered on Day 22, and 16 mg glofitamab administered on Day 43; and
   Two step up dose (SUD) cohorts:
2.5/10/16 SUD Q3W: 1000 mg obinutumzumab administered on Day 1, 2.5 mg glofitamab administered on Day 8, 10 mg glofitamab administered on Day 15, 16 mg glofitamab administered on Day 22, and 16 mg glofitamab administered on Day 43; and
2.5/10/30 SUD Q3W: 1000 mg obinutumzumab administered on Day 1, 2.5 mg glofitamab administered on Day 8, 10 mg glofitamab administered on Day 15, 30 mg glofitamab administered on Day 22, and 30 mg glofitamab administered on Day 43.

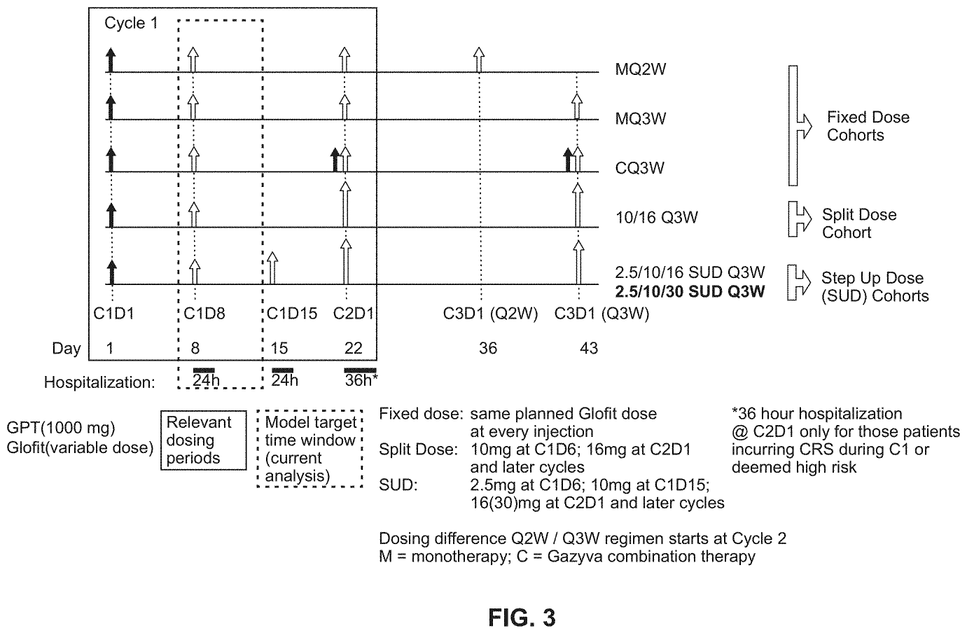

FIG. 3 represents the dosage timing in various cohorts. “Cycle 1” was defined to begin at Day 8, “Cycle 2” was defined to start at Day 22, and “Cycle 3” was defined to begin at Day 36 (Q2W regimen) or Day 43 (Q3W regimen). Thus, the monotherapy fixed-dose cohorts (MQ2W and MQ3W) and the combination-therapy fixed-dose cohort (CQ3W) did not differ across cohorts with regards to the type(s) of therapeutics administered during Cycle 1, and data could then be combined for analyses that focused on this cycle.

The 2.5/10/30 SUD Q3W cohort was used as a validation data set.

Non-Hodgkin lymphoma histologies (excluding Mantle-Cell Non-Hodgkin lymphoma histology) were further accessed.

Table 7 shows the number of subjects with complete treatment records in Cycle 1, separated by treatment regimen, and sub-type of non-Hodgkin lymphoma (aggressive, indolent, or unknown). The 2.5/10/30 SUD Q3W dose group was used as a validation data set, and subject counts for this regimen is shown in a box.
records in Cycle 1:

TABLE 7

| | | CHGROUP | | | |
|---|---|---|---|---|---|
| Dose Group | | aNHL | iNHL | Unknown | All |
| 0.6-1.0 | mg | 23 | 4 | 0 | 27 |
| 1.8-2.5 | mg | 15 | 1 | 0 | 16 |
| 4.0-10 | mg | 31 | 3 | 0 | 34 |
| 10/16 | mg | 53 | 12 | 0 | 65 |
| 2.5/10/16 | mg | 12 | 3 | 0 | 15 |
| 2.5/10/30 | mg | 35 | 14 | 2 | 51 |

| | | | | |
|---|---|---|---|---|
| All | 200 | 45 | 2 | 247 |

Flat dose cohorts: total N in mono & combo groups
Split dose cohort: 10/16 mg
SUD cohorts: 2.5/10/16 mg & 2.5/10/30 mg
iNHL: FL Grades 1-3A
aNHL: DLBCL; PMBCL; Richters Tr; Tr FL; Tr MZL

Table 8 shows how many subjects (for each treatment regimen and dosage group) had complete treatment records in Cycle 2.

TABLE 8

| | Dose Group | | | | | | | |
|---|---|---|---|---|---|---|---|---|
| C2 Completed | 0.6-1.0 mg | 1.8-2.5 mg | 4.0-10 mg | 10/16 mg | 16-25 mg | 2.5/10/16 mg | 2.5/10/30 mg | All |
| N | 3 | 6 | 3 | 0 | 2 | 0 | 3 | 17 |
| Y | 24 | 10 | 31 | 65 | 37 | 15 | 48 | 230 |
| ALL | 27 | 16 | 34 | 65 | 39 | 15 | 51 | 247 |

For each subject in represented in the training/validation data, the following data was identified in the study data, when available:
The dosage of glofitamab that was administered if a subject was in a fixed-dose cohort;
Whether the dosage of the pre-treatment agent (obinutuzumab) that was administered was less than 200 g/mL
The following laboratory variables measured/observed at Day 1 of Cycle 1 (C1D1):
   Platelet count
   Monocyte level
   Hemoglobin level
   White blood cell count (WBC)
   Fibrinogen level
   Lactic acid dehydrogenase (LDH) level
   C-reactive protein (CRP) level
   TNF-α plasma level
   Interleukin-6 (IL6) plasma level
   Aspartate aminotransferase (AST) level
   Alkaline phosphatase (ALP) level
Gz pre-Glofit (<200 g/ml)
The following clinical variables measured/observed at or prior to Day 1:
   Whether the small-cell non-Hodgkin's lymphoma was characterized as the aggressive subtype (aNHL; defined to include follicular lymphoma: Grade 1, Grade 2, or Grade 3A) or indolent subtype (iNHL, defined to include diffuse large B-cell lymphoma, primary mediastinal B-cell lymphoma, Richters transformation, transformed follicular lymphoma, transformed marginal zone lymphoma)
   Whether the subject previously had a B-cell lymphocytosis
   Whether the subject had any comorbidities
   Whether the subject had any cardiac comorbidity, including any:
      Cardiac arrhythmia (arrhythmia, arrhythmia supraventricular, atrial fibrillation, atrial flutter, atrial tachycardia, sinus bradycardia, sinus tachycardia, supraventricular extrasystoles, supraventricular tachycardia, tachycardia, tachycardia paroxysmal, ventricular extrasystoles, or ventricular tachycardia);
      Cardiac disorder, sign, and symptom NEC (cardiac disorder or hypertensive heart disease);
      Cardiac valve disorder (aortic valve stenosis or mitral valve prolapse);
      Coronary artery disorder (acute myocardial infarction, angina pectoris, arteriosclerosis coronary artery, myocardial infarction, or myocardial ischemia);
      Heart failure (cardiac failure or cardiac failure chronic);
      Myocardial disorder (cardiomyopathy, cytotoxic cardiomyopathy, diastolic dysfunction, or ischemic cardiomyopathy); or
      Pericardial disorder (pericarditis)
The following pathology-based variables measured/observed at Day 1:
   Whether bone marrow (BM) infiltration by non-Hodgkin lymphoma was detected
   Whether peripheral blood (PB) infiltration by non-Hodgkin lymphoma was detected
   Whether extranodal involvement of non-Hodgkin lymphoma was detected
   A tumor burden that identifies a sum of products of longest overall tumor diameters (SPD) and/or whether a tumor burden was equal to or greater than 3000 mm²
   An Ann Arbor lymphoma staging (and/or whether a stage is at least a Stage III)
The following demographic variable measured/observed at Day 1:
   A subject's age (and/or whether a subject was at least 64 years old)

The study monitored for and graded any Cytokine Release Syndrome occurring during or after any infusion of glofitamab, using the grading criteria set forth in Table 9 from Section III.D.2. The time at which any Cytokine Release Syndrome occurred was recorded (e.g., relative to Day 1 as defined for the respective cohort).

### IV.A.2 Data Splitting for Model Training and Validation

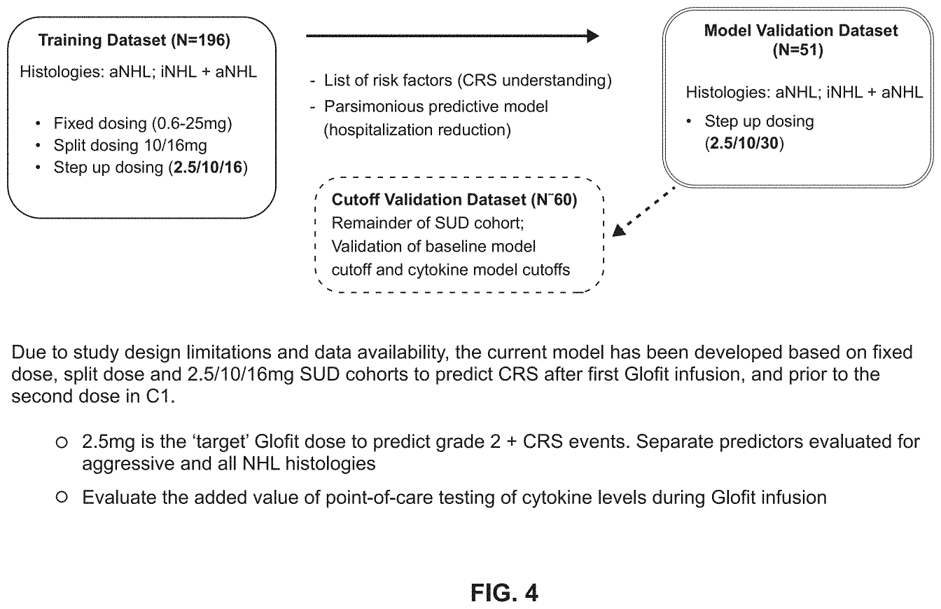

FIG. 4 shows a representation of what data was used to train and validate a feature selection model (to identify a reduced feature set and thresholds to convert any non-binary baseline characteristic into a binary variable), a risk-score generation model (to convert binary values from the reduced feature set into a risk score), and a decision-tree model (to convert the risk score and cytokine fold changes to a prediction as to whether a grade 2+ cytokine release syndrome will occur).

A challenge for a model development posed by a non-randomized or properly stratified trial (such as NP30179) is the multitude of subject sub-cohorts that are expected to display multiple confounding phenomena. The predictive or prognostic factors may be unbalanced across the treatment cohorts and subject subgroups. For example, there may be a confounding of the incidence of cytokine release syndrome with a dosage of glofitamab.

Therefore, non-overlapping training and validation data sets were used. The training data set included data corresponding to all available regimens except the target 2.5/10/30 SUD Q3W treatment regimen. The feature selection model used the training data (n=196) to identify which baseline characteristics in the training set were significantly related to whether a grade 2+ cytokine release syndrome occurred within seven days from the first glofitamab infusion. A reduced feature set was defined to include each baseline characteristic significantly related to incidence of grade 2+ cytokine release syndromes. For any baseline characteristic in the reduced feature set that has non-binary values (e.g., that has real-number values), the feature selection model further determined a weight to associate with the baseline characteristic and a threshold that most accurately separates values of the baseline characteristic that are predictive of occurrence of a grade 2+ cytokine release syndrome from other values of the baseline characteristic that are not predictive of a grade 2+ cytokine release syndrome. Separate reduced feature sets and thresholds were determined for aggressive and all non-Hodgkin lymphoma histologies.

A predictive model was defined to include a risk-score generation model to convert the reduced feature set (using associated weights and any thresholds) to a risk score and to also include a decision tree to generate subject-specific interpretable and clinically actionable outputs (e.g., recommendations as to whether to use in-patient or out-patient monitoring to monitor for potential cytokine release syndromes after treatment).

Data corresponding to the 2.5/10/30 step-up dosage (SUD) Q3W treatment regimen was defined to be the validation SUD cohort. Thus, data corresponding to subjects who received this treatment regimen were used to validate the weights of the baseline characteristics in the reduced feature set and the predictive model.

The decision tree model uses one or more thresholds (e.g., a risk threshold and a cytokine-level threshold) to predict whether a given subject is at ‘Low risk’ versus at ‘High risk’ of a cytokine release syndrome. The training data set was a combination of multiple dose regimens, not randomized or stratified, and did not involve many cases around the 2.5 mg of first dose. The current CRS mitigation strategy in 2.5/10/30 SUD also did not precisely match that of the earlier cohorts. These data-set characteristics presented challenges in fixing a classifier decision cutoff using training data set such in a manner that would result in an accurate classifier for the target SUD schedule.

### IV.A.3. Timing of Cytokine Release Syndromes

As noted in Section IV.A.1., the NP30179 study data included cytokine release syndrome data. Each cytokine release syndrome was associated with a subject, treatment regimen, severity grade, and a time metric indicating when—within a treatment regimen—the cytokine release syndrome occurred. The time metric was thus be used to determine in which treatment cycle the cytokine release syndrome occurred and an inter-cycle time of the event.

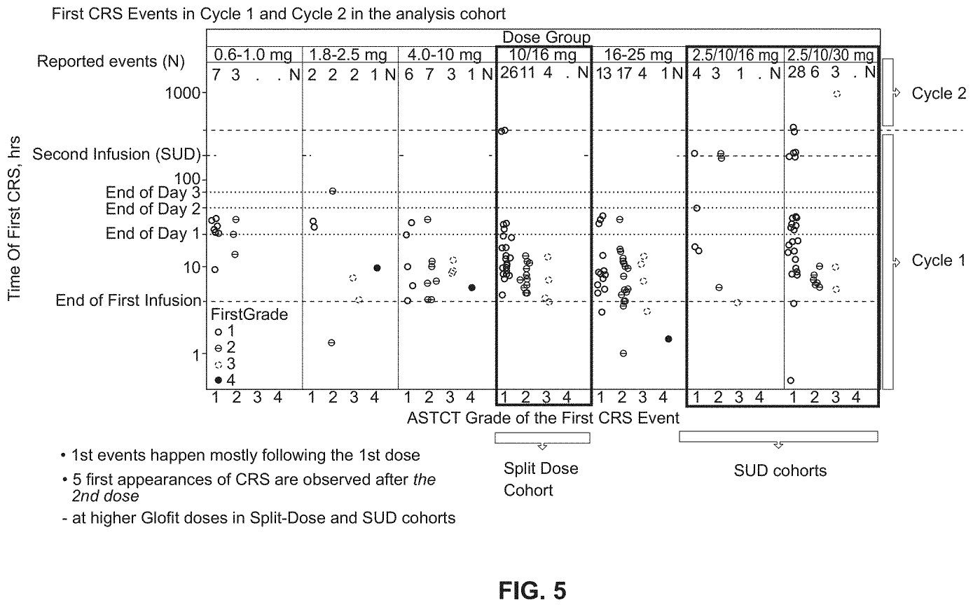

FIG. 5 shows the timing of cytokine release syndromes for each analysis cohort. Each cytokine release syndrome is represented by a symbol. If multiple cytokine release syndromes were detected for a given subject, only the first observed event is represented in FIG. 5. The position of the symbol on the (logarithmic) OY axis shows the timing of the first observed cytokine release syndrome for the subject. The severity grade was determined using the American Society for Transplantation and Cellular Therapy (ASTCT) consensus grading recommendations (as shown in Table 6, above). The severity grade of each event is represented both by the position of the symbol along the OX axis and also by the color of the symbol.

As shown in FIG. 5, the vast majority of the first appearances of cytokine release syndromes occurred during Cycle 1 of the treatment regimens. Most first events occurred within a day of the end of the first infusion in Cycle 1.

Further, the incidence of cytokine release syndromes increased as the dosages of monotherapies increased. For example, there were more than twice the number of cytokine release syndromes that occurred in the cohort that received glofitamab dosages between 10 and 25 mg (including either of the 10/16 cohort and the 16-25 cohort) relative to the cohort that received glofitamab dosages between 4 and 10 mg. Similarly, there were more than twice the number of cytokine release syndromes that occurred in the cohort that received glofitamab dosages between 4 and 10 mg relative to the cohort that received glofitamab dosages between 1 and 2.5 mg.

Cytokine release syndromes in the second cycle were not detected across any of the flat dosing monotherapy cohorts. Even in the split-dose and the step-up dose cohorts, cytokine release syndromes rarely occurred during Cycle 2. Thus, subsequent analyses presented in this Example focused on predicting cytokine release syndromes that occurred in Cycle 1.

### IV.A.4. Dose Dependency of Cytokine Release Syndromes

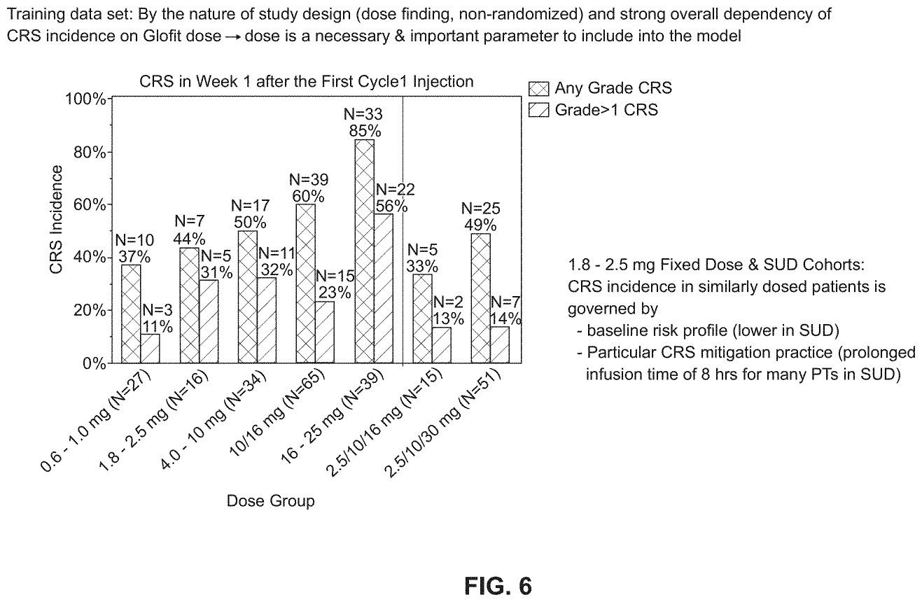

FIG. 6 shows the percentage of subjects in the training and validation data sets within each cohort that experienced a cytokine release syndrome event during the first week of Cycle 1. The blue bars correspond to any type of cytokine release syndrome. The orange bars correspond to a cytokine release syndrome of Grade 2 or higher. The incidence of cytokine release events was correlated with the dosage.

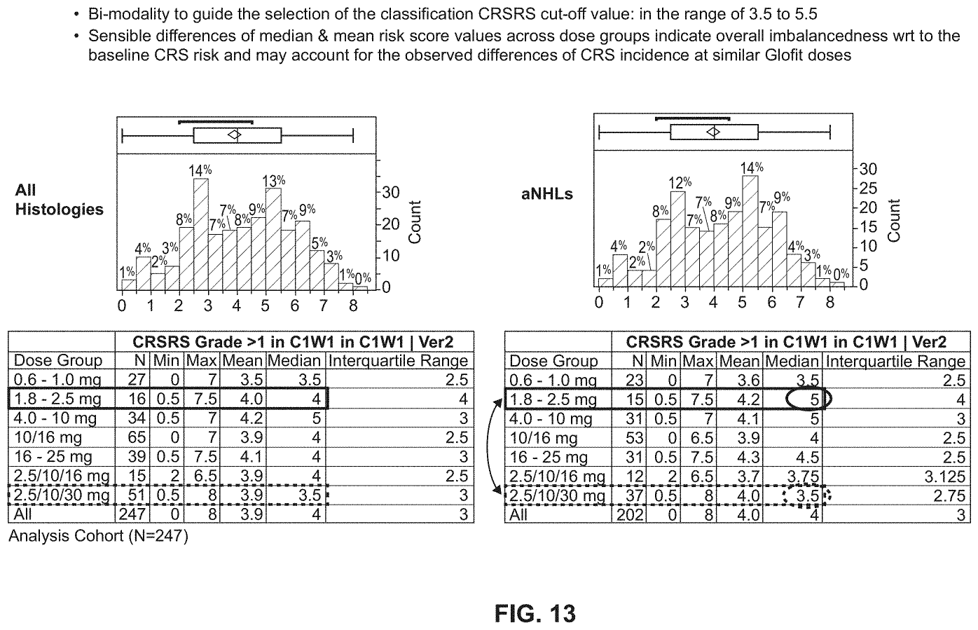

The cytokine release syndrome incidence for the 1.8-2.5-mg monotherapy cohort appeared to be different than that for the step-up dosages (which use 2.5 mg as the first dosage). This difference may reflect differences in clinical monitoring or mitigation actions (e.g., due to infusion time being prolonged for up to eight hours for many subjects in the step-up cohorts. Alternatively or additionally, the cytokine release syndrome differences may be a result of differences of these two cohorts with respect to main risk factors at baseline (with a baseline risk profiled being of lower risk for the step-up dosage cohort relative to the 1.8-2.5-mg monotherapy cohort, see also FIG. 13).

### IV.A.5. Learning Predictive Factors and Formulation of Multivariate Model

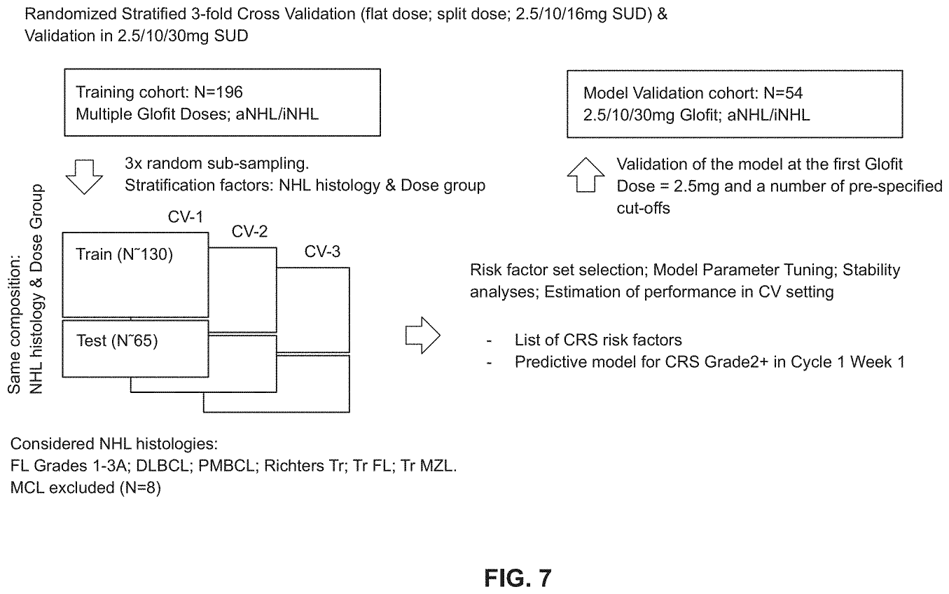

FIG. 7 shows the workflow used to identify the extent to which of various baseline characteristics (or “risk factors”) contributed to a prediction of occurrence of cytokine release syndrome and how parameters in a model were learned. The training cohort represented in this figure included each fixed-dose cohort and the 2.5/10/16 SUD Q3W split-dosage cohort. In total, 196 subjects were represented in the training cohort. The subject set included subjects who had been diagnosed with aggressive non-Hodgkin's lymphoma or with indolent non-Hodgkin's lymphoma.

The data was randomly stratified for three-fold cross validation. Stratification factors included non-Hodgkin's lymphoma histology (follicular Stage I-IIIA, diffuse large B-cell lymphoma, primary mediastinal B-cell lymphoma, Richters transformation, transformed follicular lymphoma, transformed marginal zone lymphoma). In each iteration, data corresponding to approximately 130 subjects were used for training and data corresponding to approximately 65 subjects were used for testing.

Cross-validation (using the training data) was used to select baseline characteristics predictive of the occurrence of cytokine release syndrome (by the feature selection model), to tune parameters of a regression model (by a risk-score generation model) that relates the selected risk factors to predicted probabilities of cytokine release syndrome, for stability analysis (by the risk-score generation and feature selection models), and for estimation of performance of the regression model (by the risk-score generation model). The risk-score generation model included either a bi-variate logistic regression model for CRS risk score (CRSRS) and glofitamab dose, or a multi-variate logistic regression model for baseline parameter values (same set of parameters as in CRSRS combined score) and glofitamab dose. The final set of baseline parameters and the weights of single baseline parameters in predicting cytokine release syndrome risk score has been finalized with the help of random forest and floating regression modelling. The stability of the predictors was assessed in a stratified cross-validation setting.

The 2.5/10/30 SUD Q3W split-dosage cohort was used for validation and to identify one or more clinically relevant risk-score thresholds. More specifically, the risk-score generation model was configured to output a numeric output that corresponded to a predicted probability of a cytokine release syndrome (ASTCT Grade 2+ CRS) to occur after the first dose of 2.5 mg of glofitamab. The performance of several threshold values on the predicted probability scale have been validated in the validation cohort.

Two types of analyses were performed for each of the “all histologies” data (corresponding to data where the subject had been diagnosed with either aggressive non-Hodgkin's lymphoma or indolent non-Hodgkin's lymphoma) and for aggressive non-Hodgkin's lymphoma (aNHL) data. A first analysis used a risk-score generation model to perform multiple uni-variate regressions to determine an extent to which each of multiple variables were independently predictive of whether a cytokine release syndrome of Grade 2 or higher was observed within the first week after the first glofitamab administration.

### IV.A.5.a. Uni-Variate Analyses to Assess a Degree to which Individual Variables were Predictive of Events

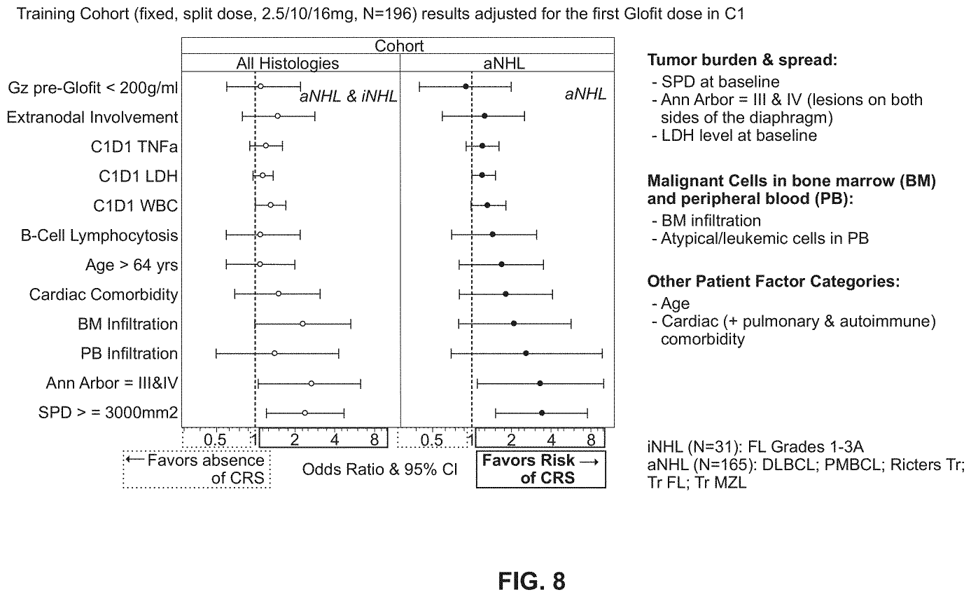

FIG. 8 is a plot that shows the degree to which each of multiple baseline characteristics was predictive of occurrence of cytokine release syndrome (of Grade 2+ after the first glofitamab dosage). The dose-adjusted predictive strength of each baseline characteristic is provided in terms of Odds Ratio per unit change of factor level. Confidence intervals (not adjusted for multiple testing) help to interpret significance. The odds ratio represents a degree to which a given characteristic is predictive as to whether a cytokine release syndrome occurred. Larger odds ratios are indicative of an increased risk of a cytokine release syndrome at the indicated factor level. The odds-ratio statistic considers the predictiveness of a single variable in isolation.

Additional input was provided by random forest and floating (multivariate) regression experiments. For any feature in the reduced feature set that corresponded to a non-binary (e.g., real-number) variable, the feature was defined to be a binary value that indicated whether a particular inequality was established using the non-binary variable (e.g., whether a given real number was less than a threshold set for the feature).

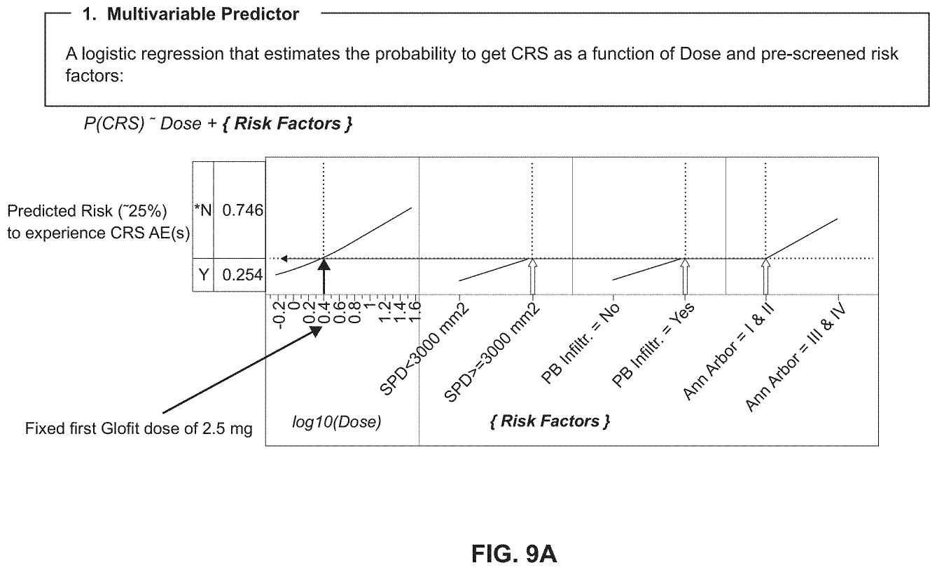

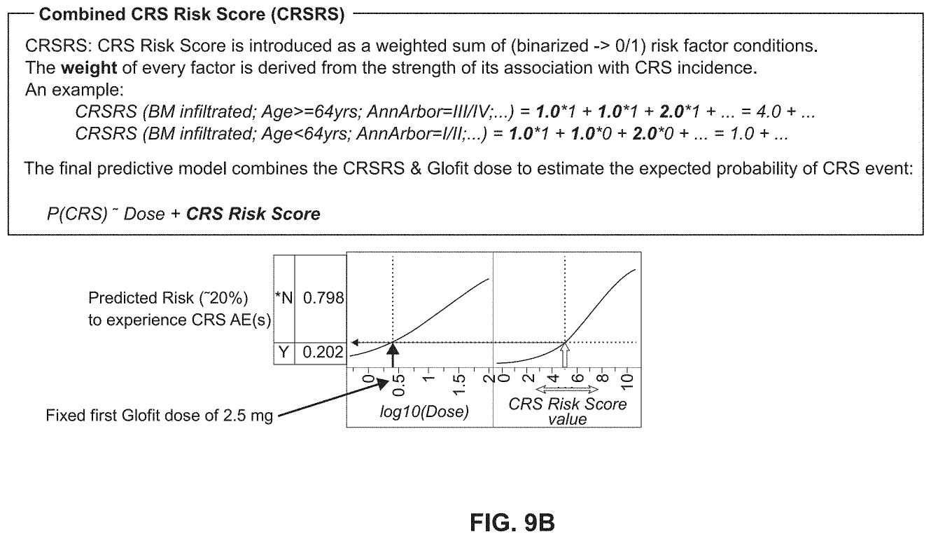

FIG. 9A illustrates how a multivariate logistic regression models can be used to predict the cytokine release risk. The figure illustrates the output of the model, the predicted probability of Grade 2+ cytokine release syndrome as function of glofitamab dose (first predictive factor), tumor burden (SPD categorized), peripheral blood infiltration status, and Ann Arbor stage category (I or II vs III or IV). At every particular combination of these parameters the model predicts a certain risk of Grade 2+ cytokine release syndrome. At 2.5 mg of glofitamab and particular values of other baseline parameters (indicated by vertical dashed lines & red arrows), the predicted risk was estimated to be about 25%.

### IV.A.5.b. Combined Cytokine Release Syndrome Risk Score (CRSRS) for Predicting Events

A combined cytokine release syndrome risk score (CRSRS) was defined to be a weighted sum of the selected (the reduced feature set) subject characteristics at baseline. The risk-score generation model defined the weights as those that maximized classification accuracy and stability in the training data. Each weight was primarily derived from log (Odds Ratio) in the univariate dose-adjusted logistic regression that predicts odds ratio of Grade 2+ CRS from the dose and the baseline value of the corresponding parameter. The weight was further tuned by including information about the feature's stability as in the random forest and floating feature selection experiments. (See FIG. 7.)

In this Example, the risk-score generation model 184 was defined to generate a cytokine release syndrome risk score, which predicts a probability of a Grade 2+ cytokine release syndrome occurring at a particular glofitamab dosage for a particular subject. A risk of a subject experiencing cytokine release syndrome was determined based on the cytokine release syndrome risk score and a treatment dosage.

In this Example, the decision-tree model included a classifier configured to translate a real-number cytokine release risk score into a binary prediction as to whether a cytokine release syndrome would occur based on whether the sum of the cytokine release risk score and dosage exceeded a risk-score threshold. The cytokine release risk score was defined to have a minimum value of 0 and a maximum value of 8.5.

FIG. 9B illustrates how the cytokine release risk score is calculated and used, together with the dosage in the predictive model. As illustrated in FIG. 9B, a slope of a plot relating the incidence of Grade 2+ cytokine release syndromes to the cytokine release risk score can be steeper than a slope of a plot relating the incidence of Grade 2+ cytokine release syndrome to the dosage.

Table 9 shows the final weights assigned to baseline characteristics (or binary transformations thereof) contributing to the cytokine release syndrome risk score. The characteristics associated with the highest weights indicated whether an Ann Arbor Stage was at least III and whether a sum of products of longest overall tumor diameters across tumors was at least 3000 mm2. The characteristics associated with intermediate weights indicated whether a subject was older than 64, whether bone marrow infiltration was observed, and whether atypical cells were detected in peripheral blood. The characteristics associated with the lowest weights indicated whether a subject had a cardiac comorbidity, whether a white blood cell count was greater than 4.5*10\hat{ }9 cells/l and whether lactic acid dehydrogenase was greater than 280 U/l.

TABLE 9

| Parameter & Cutoff | Weight |
|---|---|
| Lactate Dehydrogenase (LDH) > 280 U/l | 0.5 |
| White blood cell count (WBC) > 4.5 * 10⁹ Cells/l | 0.5 |
| Age > 64 yrs | 1 |
| Cardiac comorbidity | 0.5 |
| Bone marrow infiltration | 1 |
| Atypical cells in peripheral blood | 2 |
| Ann Arbor Stage = III or IV | 2 |
| Sum of Product of Tumor Diameters (SPD) >= 3000 mm² | 2 |

### IV.A.6. Performance in the Training and Validation Data Sets

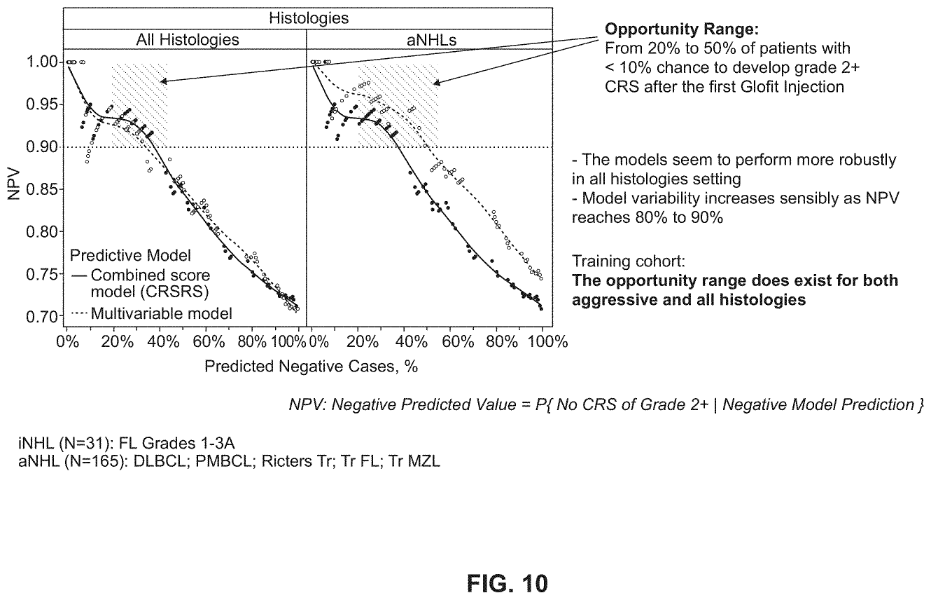

FIG. 10 shows negative predicted values (NPV) relative to the predicted negative cases corresponding to risk scores from two versions of the risk-score generation model. In one case, the risk-score generation model transformed binary versions of baseline characteristics represented in the reduced feature set into a combined cytokine release syndrome risk score (CRSRS, blue line. See also FIG. 9B). In another case, the risk-score generation model transformed raw baseline characteristics represented in the reduced feature set into a multivariate model output (orange line. See also FIG. 9A).

Every point in FIG. 10 corresponds to a distinct cut off (e.g., used by a decision-tree model), where values above the cut off were then considered as corresponding to predictions that a cytokine release syndrome of at least grade 2 occurred and values below the cut-off were considered as contrary predictions. For each of a set of cut offs, the negative predicted value and predicted negative case percentage were recorded for the cut off.

In FIG. 10, the OX coordinate indicates the negative call rate at that cut-off, which is the proportion of cases from the data set classified as low Risk′ by the decision tree model. The OY coordinate of each point identifies the negative predicted value at the point-associated cut-off. The negative predicted value is the probability that a subject classified as low risk will indeed not develop a cytokine release syndrome of Grade 2 or above. The shaded area in FIG. 10 is the opportunity range, where between 20-50% of subjects had less than a 10% chance of developing a cytokine release syndrome of Grade 2 or above after the first glofitamab dose.

As illustrated by FIG. 10, the model variability increases sensibly as the negative predictive value reaches 80-90%. The opportunity range exists for both the “all histologies” and aggressive non-Hodgkin's lymphoma data.

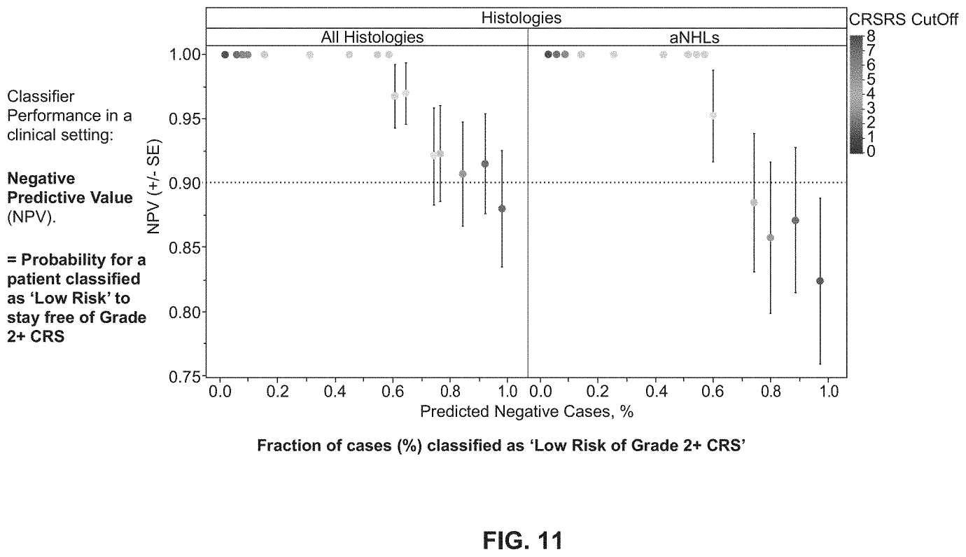

To characterize the performance in predicting cytokine release syndromes (of Grade 2+ following a first glofitamab dose) for the target 2.5/10/30 SUD cohort, the first glofitamab dose was defined to be 2.5 mg. FIG. 11 shows the negative predictive value versus predicted negative cases for the model validation data set of the 2.5/10/30 mg step-up dosage cohort. Each dot corresponds to a different threshold used by the decision tree model to convert a risk score to a binary prediction of whether a cytokine release syndrome (of Grade 2 or above after the first glofitamab dose) will occur. If a sum of the risk score and dosage is lower than the threshold, the classifier generated a ‘Low Risk’ result corresponding to a prediction that the cytokine release syndrome would occur. Thus, the percentage of predictions corresponding to ‘Low Risk’ results (corresponding to a prediction that a cytokine release syndrome of Grade 2+ will occur following a first glofitamab dose) increases as the threshold increases.

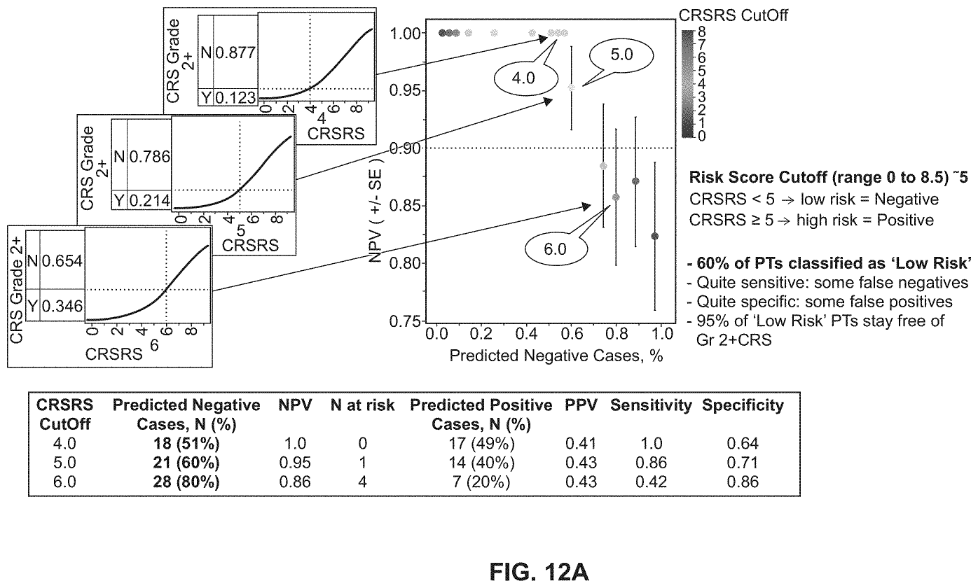

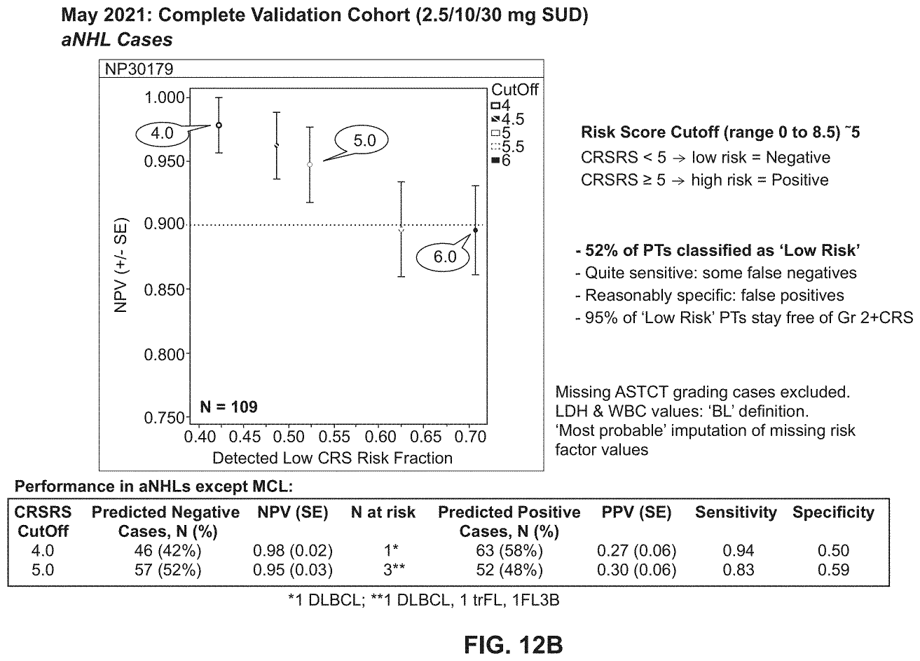

FIG. 12A shows the probability of a cytokine release syndrome (of Grade 2 or above after the first glofitamab dose) occurring as a function of the cytokine release syndrome risk score (CRSRS) for each of three exemplary thresholds that differentiate whether it is a predicted that the event would have or would not have occur. Low thresholds corresponded to predicting that more of these events would have occurred.

The table in FIG. 12A shows how the predicted number of positive cases decreases from 17 (49%) to 7 (20%) as the threshold increases from 4.0 to 6.0. Data for a sub-population of subjects that were predicted to be low risk and to not have experienced to experience the cytokine release syndrome (of Grade 2 or above after the first glofitamab dose) was further examined.

When a cut-off threshold of 4.0 was used, the decision tree model predicted that 51% of the subjects would not experience the cytokine release syndrome (of Grade 2 or above after the first glofitamab dose), and no such events were observed for subjects having a score below the threshold, such that the observed Negative Predictive Value was 1.0. When a cut-off threshold of 6.0 was used, the decision tree model predicted that 80% of the subjects would not experience the cytokine release syndrome (of Grade 2 or above after the first glofitamab dose), but—for 14% of the below-threshold subjects—an event was indeed observed (such that the Negative Predictive Value was 0.86). When a cut-off threshold of 5.0 was used, the decision tree model predicted that 60% of the subjects would not experience the cytokine release syndrome (of Grade 2 or above after the first glofitamab dose), and such events were observed for only 5% of the subjects. It appeared as though a cutoff near 5.0 was optimal for differentiating between positive and negative cases. Of the sub-population of subjects corresponding to results that were below the threshold of 5.0, 95% of the subjects predicted to be at low risk for experiencing a cytokine release syndrome (of Grade 2 or above after the first glofitamab dose) in fact did not experience such as event.

The trained CRSRS model was further used to generate CRS risk predictions in the complete (model and decision cutoff) validation set. Each subject in the validation set had been diagnosed with NHL and was a participant in the NP30179 clinical study. Each score was compared to one of two thresholds (4.0 or 5.0) to generate binary predictions as to whether the subject experienced cytokine release syndrome of Grade 2 or above after administration of a first glofitamab dose. The validation set included date from 156 subjects. Data from the analysis is shown in FIG. 12B.

As shown in the plot in FIG. 12B, the CRSRS thresholds remain positively correlated with the percentage of predicted negative cases and negatively correlated with the negative predictive values. As shown in the table, when a cut-off threshold of 4.0 was used to assess the validation data, the trained decision tree model predicted that 42% of the subjects would not experience the cytokine release syndrome (of Grade 2 or above after the first glofitamab dose), and this was accurate for 98% of the subjects having a score below the threshold (resulting in a Negative Predictive Value of 0.98). (The detected fraction of subjects who did not experience a cytokine release syndrome of Grade 2 or above was 40%). The standard error was 0.02, and the confidence interval was 0.92 to 0.99.

When a cut-off threshold of 5.0 was used to assess the validation data, the trained decision tree model predicted that 52% of the subjects would not experience the cytokine release syndrome (of Grade 2 or above after the first glofitamab dose), and this was accurate for 98% of the subjects having a score below the threshold (resulting in a Negative Predictive Value of 0.98).

Notably, the predicted percentages of negative cases determined using the training data (and shown in FIG. 12A) is very similar to those determined using the validation data (shown in FIG. 12B).

Further, with respect to the validation set, the association between the CRSRS at baseline and a grade of any observed cytokine release syndrome occurring after the first infusion of glofitamab was observed for both CART-naïve and experienced subjects, as well as those pretreated with dexamethasone or other corticosteroids.

### IV.A.7. Distribution and Properties of Cytokine Release Syndrome Risk Score

FIG. 13 shows the distribution of baseline cytokine release syndrome risk score (CRSRS) values corresponding to clinical study NP30179. As shown, the distribution is multi-modal, with modes around 2.3, 5.6, and 5.6.

Further, the baseline risk may differ across cohorts, which may explain some of the differences between observations of cytokine release syndrome between subjects who received a same treatment dosage. This detection discrepancy may explain much of the observed difference in cytokine release syndrome incidence in these cohorts. The tables shown in FIG. 13 summarize the distribution statistics of the cytokine release syndrome risk scores in these dose group (of Grade 2 or above after the first glofitamab dose). (FIG. 6 shows a summary of cytokine release syndrome incidence in Cycle 1 after the first glofitamab infusion.)

### IV.B. Example 2: Exemplary Analysis of how Early Changes of Cytokine Levels May be Predictive of the Incidence and Severity of Cytokine Release Syndrome

For each of a set of subjects in the NP30179 study (who were also within the cohorts used for training, as indicated in the box in FIG. 4), cytokine data was collected and analyzed to determine cytokine kinetics and the extent to which various types of cytokine levels were predictive of the incidence and/or severity of cytokine release syndromes. Each subject in the set of subjects had been diagnosed with non-Hodgkin's lymphoma and were within fixed dose cohorts of NP30179, receiving a fixed dose of glofitamab on Day 8 of the study, following Gpt on C1D1, as indicated in FIG. 3. Table 10 shows a breakdown of the set of subjects, based on each of the dosage of glofitamab and also based on the sub-type of non-Hodgkin's lymphoma with which the subject had been diagnosed (aggressive or indolent).

TABLE 10

| | | CHGROUP | |
|---|---|---|---|
| Dose Group in C1 | TRT02P | aNHL | iNHL |
| 0.6-1.0 mg | RO7082859 600 UG | 15 | 14 |
| | RO7082859 1000 UG | 7 | 0 |
| 1.8-2.5 mg | RO7082859 1800 UG | 8 | 1 |
| | RO7082859 4000 UG | 1 | 0 |
| 4.0-10 mg | RO7082859 4000 UG | 11 | 2 |
| | RO7082859 10000-16000 UG | 2 | 0 |
| | RO7082859 100000 UG | 13 | 0 |
| 16-25 mg | RO7082859 16000 UG | 20 | 7 |
| | RO7082859 25000 UG | 5 | 0 |

For each dosage range, Table 11 shows a distribution as to a duration of the first glofitamab administration. As shown, most of the infusions occurred over 4 hours.

TABLE 11

| Dose | | Infusion Duration | | | |
|---|---|---|---|---|---|
| Group | | C1D8, h | | | |
| in C1 | TRT02P | 2 | 3 | 4 | 5 |
| 0.6-1.0 mg | RO7082859 600 UG | 0 | 0 | 19 | 0 |
| | RO7082859 1000 UG | 0 | 0 | 7 | 0 |
| 1.8-2.5 mg | RO7082859 1800 UG | 0 | 0 | 8 | 1 |
| | RO7082859 4000 UG | 0 | 0 | 1 | 0 |
| 4.0-10 mg | RO7082859 4000 UG | 0 | 0 | 12 | 1 |
| | RO7082859 10000-16000 UG | 0 | 0 | 2 | 0 |
| | RO7082859 100000 UG | 1 | 0 | 11 | 1 |
| 16-25 mg | RO7082859 16000 UG | 0 | 2 | 24 | 1 |
| | RO7082859 25000 UG | 0 | 1 | 3 | 1 |

### IV.B.1. Exemplary Cytokine Dynamics

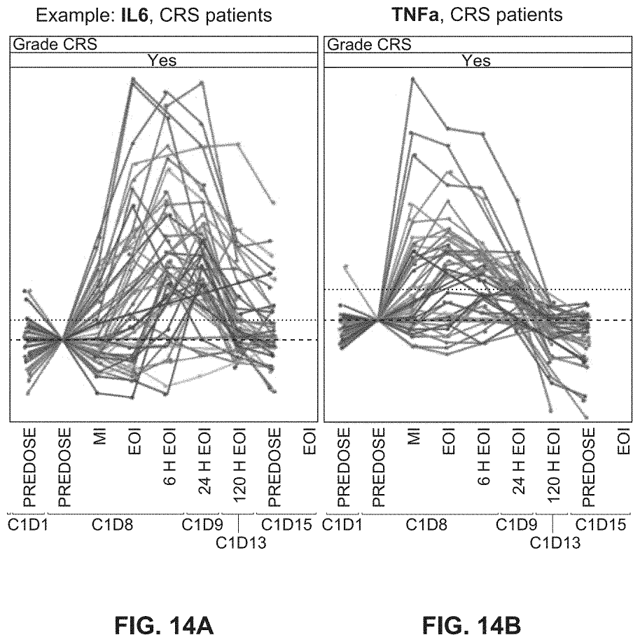

FIGS. 14A and 14B show the fold changes of IL-6 and TNF-α (respectively) during a first glofitamab treatment cycle. Each line corresponds to a different subject who experienced a cytokine release syndrome (of any grade). The first x position corresponds to pre-dose of Gpt on C1D1. The second x position corresponds to pre-dose of glofitamab on C1D8. All subjects' cytokine-level data were normalized to the second time point, collected prior to the first administration of glofitamab. The third x position (MI) corresponds to the middle of the glofitamab infusion. The fourth x position (EOI) corresponds to the end of the glofitamab infusion. The fifth, sixth, and seventh x-positions (6 H EOI, 24 H EOI, and 120 H EOI) correspond to 6, 24, and 120 hours (respectively) after the end of the glofitamab infusion.

Peaks in both cytokines were observed after initiation of treatment. With respect to IL-6, the peaks started developing at the end-of-infusion (EOI) time point. With respect to TNF-α, the peaks started developing even earlier at the mid-infusion (MI) time point.

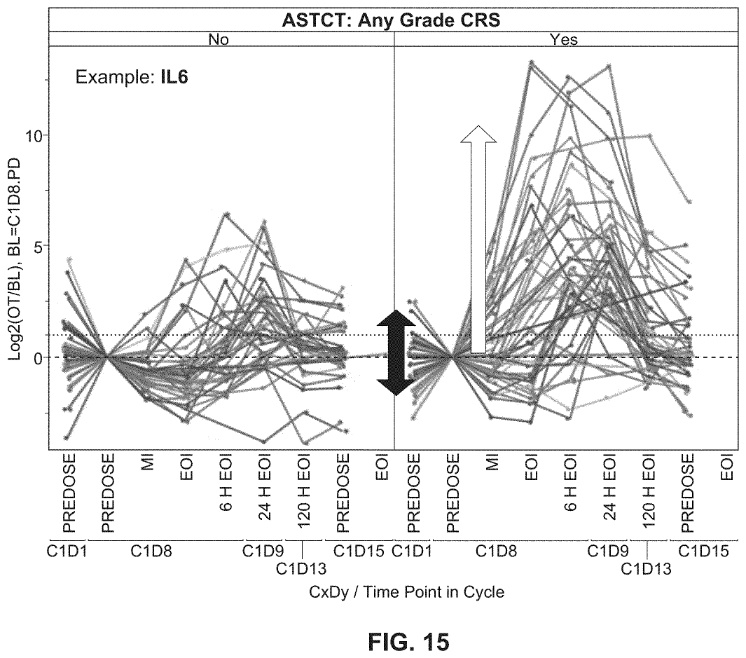

FIG. 15 contrasts the cytokine fold changes of IL-6 for subjects who did not experience a cytokine release syndrome (left plot) and for subjects who did experience a cytokine release syndrome (right plot). Notably, aside from the arrows, the right plot in FIG. 15 is the same as FIG. 14A.

An “on-treatment” (OT) time point was defined to include the mid-infusion time point and the end-of-infusion time point, and a “baseline” (BL) time was defined to be the time point before treatment began (C1D8.pre-dose). As shown in FIG. 15, the fold change of IL-6 at time points following initiation of treatment (e.g., MI, EOI, 6 H EOI, etc.) are generally substantially positive across subjects who experienced a cytokine release syndrome (right graph, compare the change on treatment—the green arrow—with the variability pre-Glofit—the red arrow), while this association was not observed across subjects who did not experience a cytokine release syndrome (left graph).

For each subject, an on-treatment cytokine fold change was calculated defined to be:

log₂(1+OT)−log₂ (1+BL)

where OT is a maximum cytokine level (in picograms per milliliter) during the on-treatment time period and BL is a cytokine level (in picograms per milliliter) during the baseline time point. These on-treatment cytokine fold changes were then segregated based on whether a cytokine release syndrome occurred for the subject and the grade of any observed cytokine release syndrome.

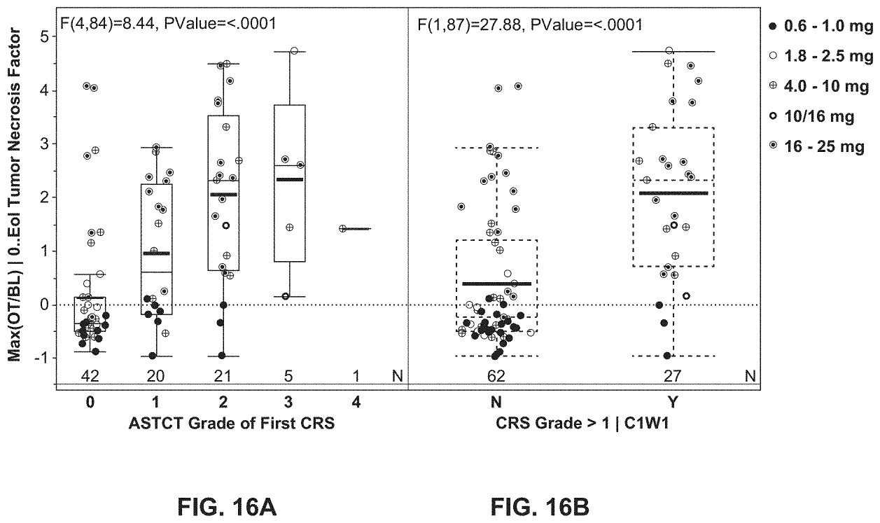

FIGS. 16A-16B shows box plots indicating how on-treatment TNFa cytokine fold changes depended on the existence or grade of the first cytokine release syndrome. Each point represents a subject. Each point is color-coded to indicate a dosage of glofitamab received by the subject.

In FIG. 16A, an x-value of 0 indicates that no cytokine release syndrome was observed. Each non-zero x-value indicates a grade of an observed cytokine release syndrome. In FIG. 16B, the data points were separated based on whether a cytokine release syndrome of a grade of at least 2 was observed.

As shown, the on-treatment cytokine fold change increased across grades of a first cytokine release syndrome event (left plot) and differed across groups defined based on whether a cytokine release syndrome of a grade of at least 2 was observed was observed (right plot). Specifically, the on-treatment cytokine fold change was higher for higher grades of cytokine release syndromes.

While the on-treatment cytokine fold change captures the difference between the log of a maximum cytokine level plus one and the log of a baseline cytokine level plus one, other cytokine fold changes may be calculated that represent differences between a cytokine level at any time point and a cytokine level at a baseline time point. That is, a cytokine fold change can be defined as

log₂(1+T)−log₂(1+BL)

where T is a cytokine level (in picograms per milliliter) during the any time period and BL is a cytokine level (in picograms per milliliter) during the baseline time point.

If baseline cytokine levels are associated with cytokine release syndrome, a relationship between cytokine levels and incidence of cytokine release syndromes may not be captured by evaluating cytokine fold changes. However, this cytokine fold change metric may facilitate characterizing within-subject changes and reducing inter-subject variability. This cytokine fold change metric may further facilitate capturing the pharmaco-dynamic concept of induction. Accordingly, subsequent cytokine-level analyses in this Example focus on the cytokine fold change metric (or the on-treatment cytokine fold change).

The cytokine fold change can reflect a pharmaco-dynamic concept of induction by treatment. Absolute fold changes may better compensate for baseline variability across subjects, so as to convey cytokine kinetic characteristics.

### IV.B.3. Effect of Dose on Early Cytokine Changes and Association of Cytokine Release Syndrome

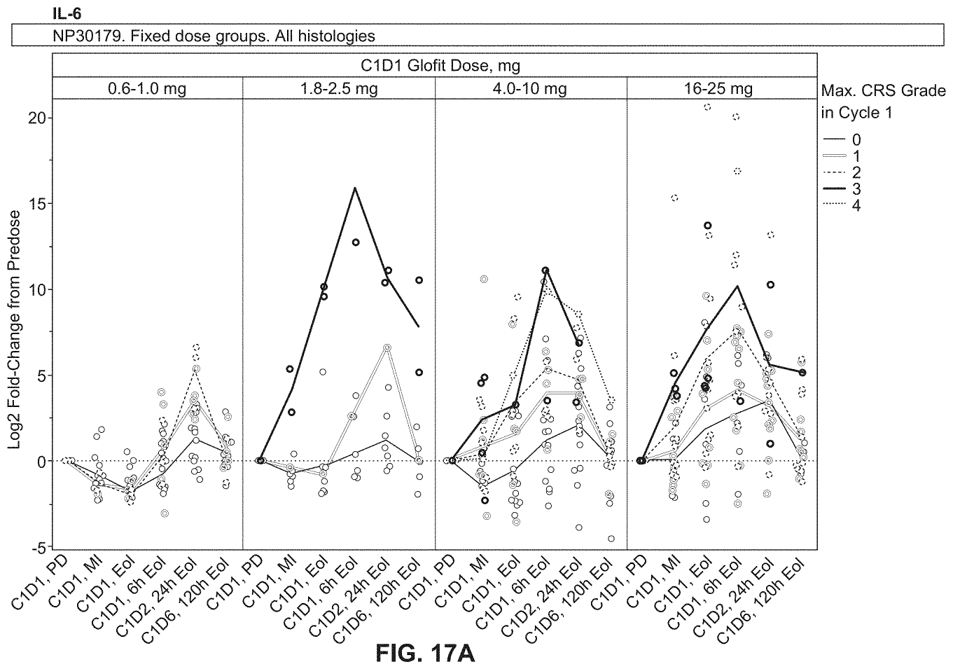

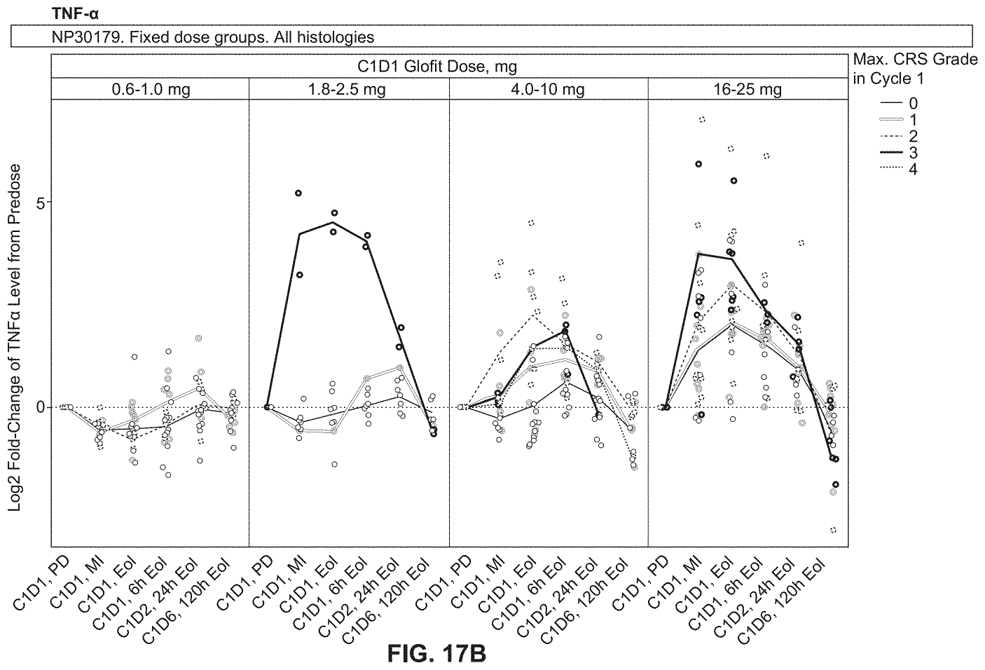

FIGS. 17A-17B show how the on-treatment levels of each of two cytokines (IL-6, TNF-α) change across the first cycle of glofitamab treatment. The four subplots shown in each figure correspond to four different dosage ranges. Each symbol represents a subject. A color of the symbol indicates whether the subject had a cytokine release syndrome and, if so, a grade of the event. For each incidence and severity of cytokine release syndromes and for each dosage range, average cytokine fold changes were also calculated using the cytokine levels of subjects associated with the incidence/severity and with the dosage range. These average values are shown in via solid lines in FIGS. 17A-17B.

These figures show that there is a clear dependency between the dosage of glofitamab administered and the level of cytokine induction. That is, the peak magnitudes of cytokine fold changes are larger when higher dosages of glofitamab were administered.

Further, the magnitude of peaks of cytokine fold changes is correlated with the severity of cytokine release syndromes. That is, higher grades of cytokine release syndromes (e.g., represented in purple or red lines) are associated with higher peak cytokine levels.

Additionally, with respect to IL-6, TNF-α, and IL-8, the magnitude of peak cytokine levels is correlated with the timing of peak cytokine levels. More specifically, higher peak cytokine levels (and cytokine release syndromes of greater severity) are associated with earlier peak times.

With respect to subjects who did not experience a cytokine release syndrome, a dependency between the on-treatment cytokine fold change of IL-6, TNF-α, IL-8 and IL-10 cytokines and the dosage was seen at dosages exceeding 4 mg glofitamab. A dependency between the on-treatment cytokine fold change of MIPb and the dosage was seen at dosages exceeding 2 mg glofitamab. The average of the subject-specific on-treatment cytokine fold changes at a 10 mg dosage of glofitamab were 1.5-fold for IL-6, 2-fold for TNF-α, 1.5-fold for IL-8, 4-fold for MIPb, and 8-fold for IL-10. The average of the subject-specific on-treatment cytokine fold changes at a 20 mg dosage of glofitamab were: 16-fold for IL-6, 8-fold for TNF-α, 4-fold for IL-8, 100-fold for MIPb, and 100-fold for IL-10.

With respect to subjects who did experience a cytokine release syndrome, a dosage dependency began at even the lowest dosages of glofitamab. On-treatment cytokine fold changes of IL-6 were as large as 30-1000.

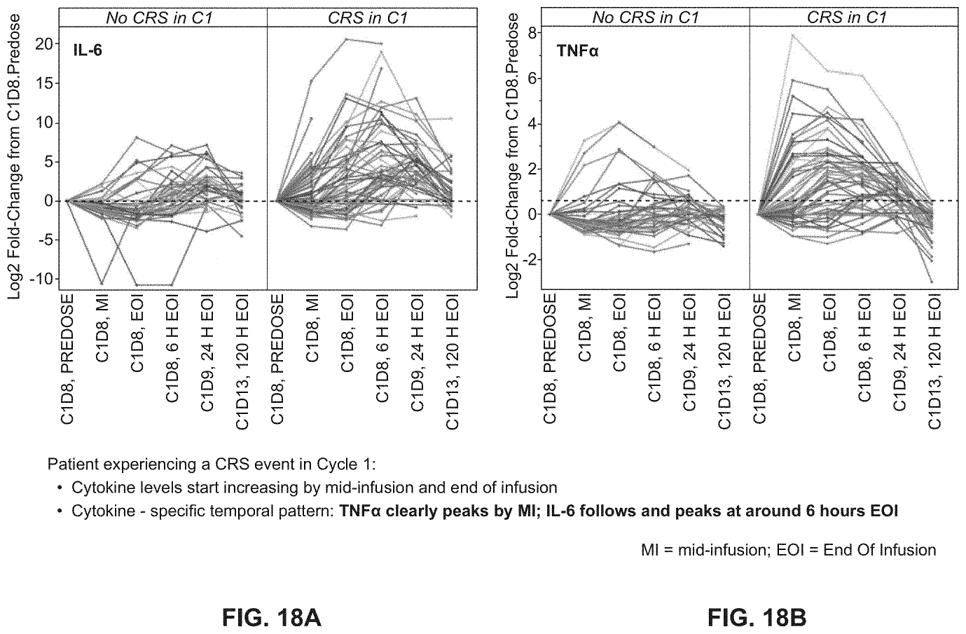

FIGS. 18A-18B show the maximum log 2 fold-changes across subjects for IL-6 and TNF-α, respectively. The data was separated based on whether any non-zero grade of cytokine release syndrome was observed in the first cycle of treatment. In each of FIGS. 18A-18B, each line in the left subplot corresponds to a subject for which no cytokine release syndrome was observed in the first cycle, while each line in the right subplot corresponds to a subject for which a cytokine release syndrome was observed during the first cycle. These line plots show that the highest peaks of TNF-α are observed at the middle-of-infusion (MI) time point. Meanwhile, the highest peaks of IL-6 occur at the end of infusion (EOI) or 6 hours afterwards.

### IV.B.4. Timing of Effect of Dose on Early Cytokine Changes and Association of Cytokine Release Syndrome

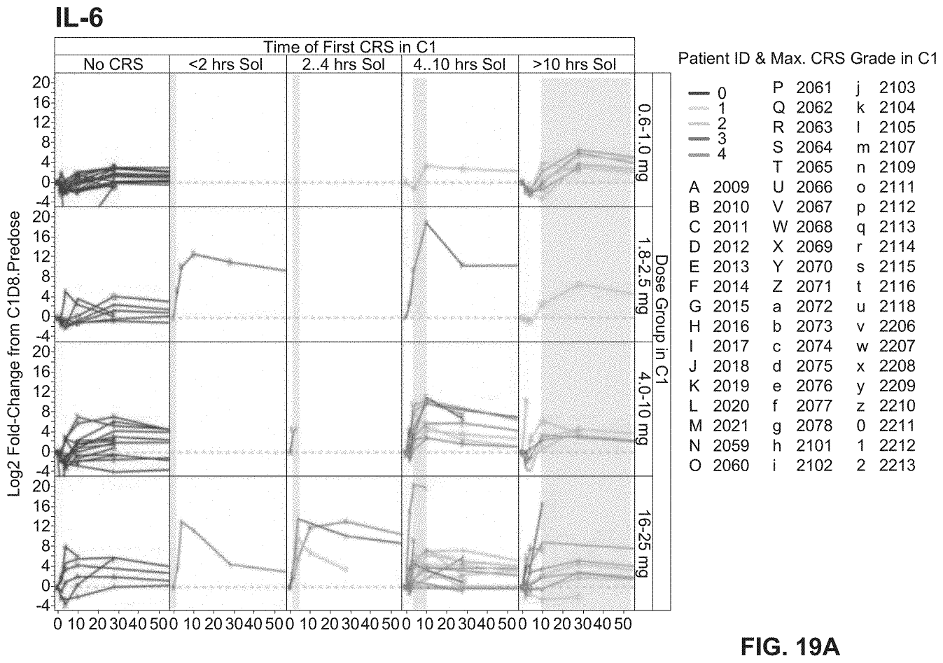

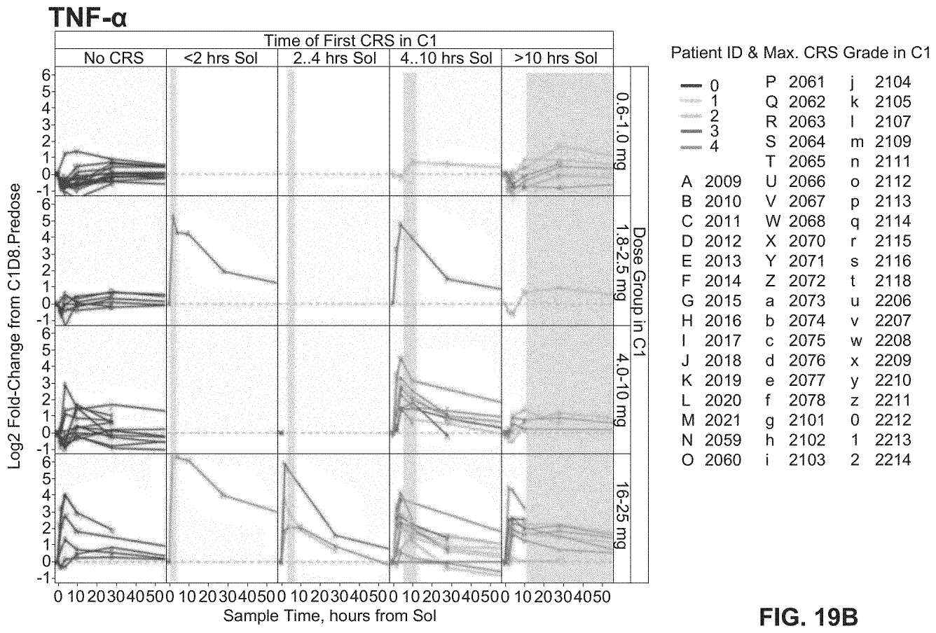

To investigate the extent to which dynamics of cytokine levels are associated with dynamics of cytokine release syndrome incidences, FIGS. 19A-19B were generated to show time courses of cytokine fold changes, while stratifying subjects according to time of onset of cytokine release syndrome relative to treatment initiation.

The columns correspond to different timing of any first cytokine release syndromes. Specifically, the first column corresponds to instances where no cytokine release syndrome occurred. The second, third, fourth, and fifth columns correspond to instances where a cytokine release syndrome occurred less than 2 hours from a start of the treatment infusion, between 2-4 hours from a start of infusion, between 4-10 hours from a start of infusion, and more than 10 hours from a start of infusion, respectively.

The different rows correspond to different glofitamab dosages. Lower rows correspond to higher dosages.

Each line corresponds to a single subject and shows the cytokine fold change of a given cytokine across time (beginning at the start of infusion). The color of the line represents a grade of cytokine release syndrome (with dark green lines representing that no such event occurred).

The timing of cytokine release syndrome onset is indicated within the shaded regions. Thus, cytokine fold changes that are above zero within the unshaded regions precede cytokine release syndrome onset and may serve as an indicator of impending cytokine release syndrome.

With respect to IL-6 (FIG. 19A), positive fold changes were detected in advance of cytokine release syndrome presentation in some, but not all subjects. With respect TNF-α (FIG. 19B), the vast majority of instances where a subject experiences a cytokine release syndrome were associated with a peak in the fold change of the cytokine during the time window preceding cytokine release syndrome onset, particularly for glofitmab dosages that exceeded 1 mg.

A more focused analysis was performed that only processed data corresponding to first glofitamab dosages between 1.8 and 10 mg so as to minimize the observed effect of dosage of cytokine induction. Further, to assess the accuracy of predictions, a “true” prediction (of a cytokine release syndrome occurring) was recorded when the cytokine fold change was greater than 0 for a time point before or 4.0 from the start of infusion, and a “false” prediction was other otherwise recorded.

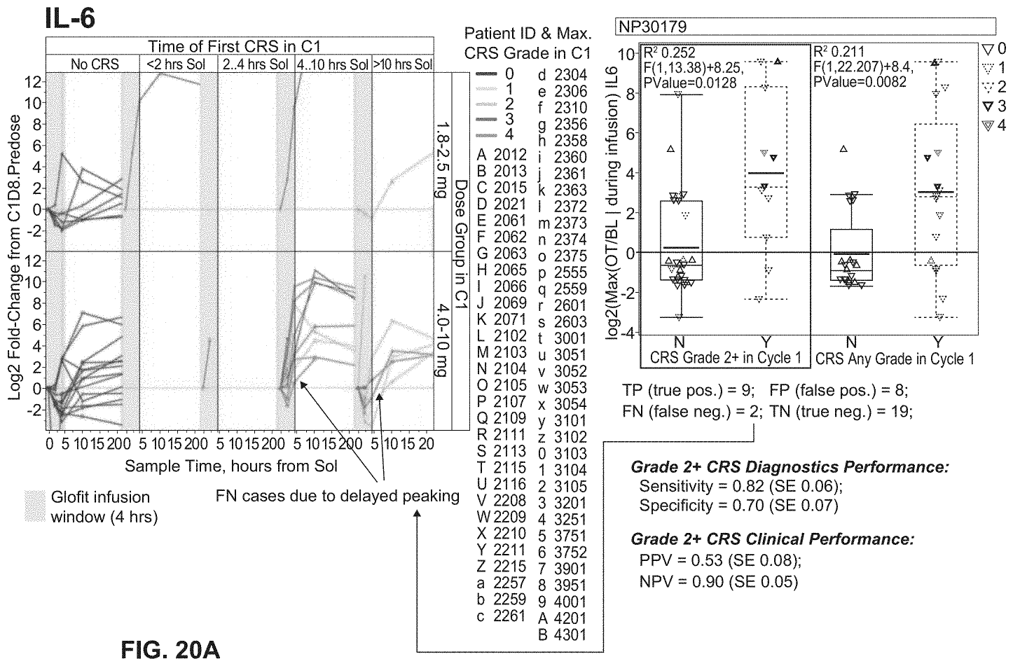

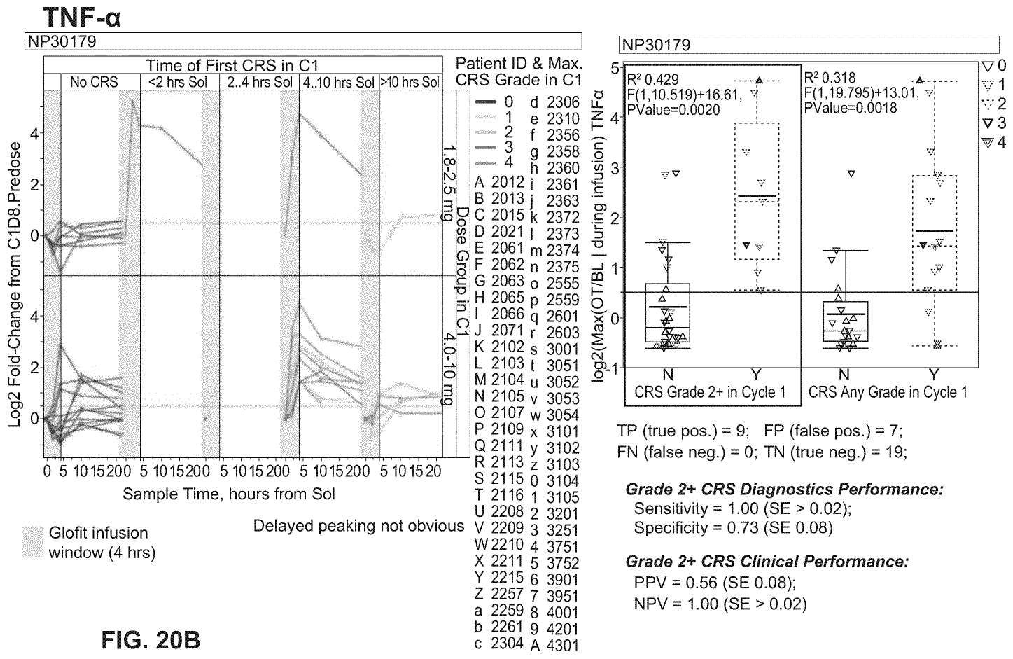

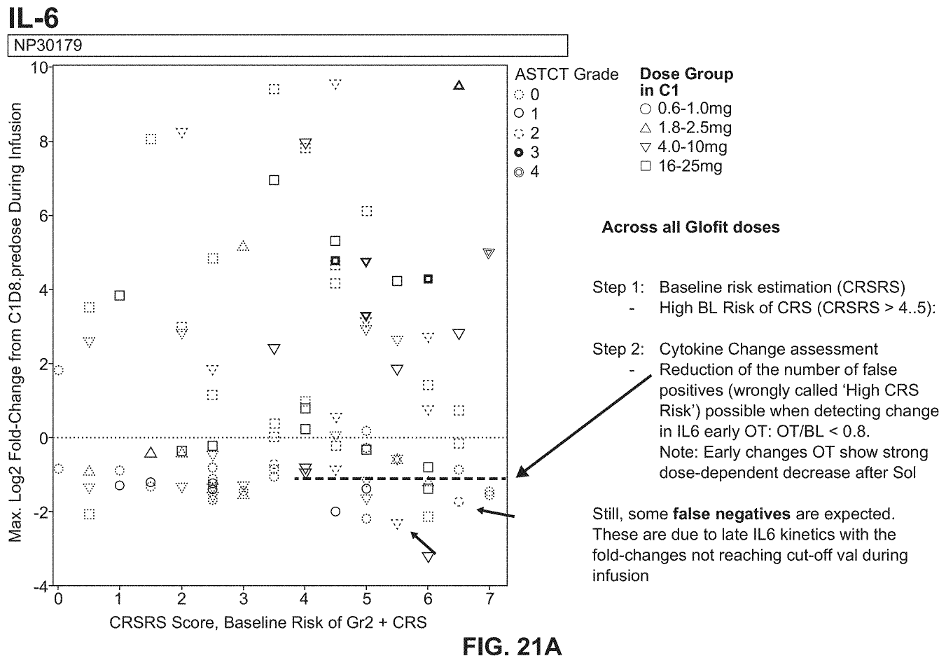

With respect to each of FIGS. 20A-20B, the left subplots show a subset of the data (corresponding to the 1.8-10 mg dosage range) shown in FIGS. 19A-19B. As illustrated, in multiple cases, the cytokine fold change of the cytokine did not cross the y=0 line (and thus did not represent an increase in the cytokine level relative to baseline) with the four-hour administration period.

The right subplots show boxplots that compare the cytokine fold change of the cytokine levels across instances differentiated based on whether any type of cytokine release syndrome occurred or based on whether a cytokine release syndrome of at least Grade 2 occurred. These plots show that the cytokine levels are higher for instances where a cytokine release syndrome occurred (generally or that was of at least Grade 2).

The true positive, false positive, true negative and false negative statistics are further shown in FIGS. 20A-20B. The predicted event occurrence was based on whether the cytokine log 2 fold change exceeded zero across an x-axis range of ≤4 hours.

The presented data indicates that, across cytokines, the true positives surpass the false positives and that the true negatives surpass the false negatives. Further, the sensitivity, specificity, positive predictive values, and negative predictive values were nearly all above chance (>0.5) across cytokines.

### IV.B.5. Association Between Cytokine Release Syndrome Risk Score and Change of Cytokine Level

As described herein, the fold change of various cytokines is predictive of the incidence and severity of cytokine release syndrome occurrence. Further, as illustrated in Example 1 (e.g., see the “Risk Score” results in FIG. 8), the cytokine release syndrome risk score (CRSRS) was also predictive of the incidence.

Potentially, the predictiveness of the fold-change cytokine levels is partly or fully redundant with the predictiveness of the cytokine release syndrome risk score. Alternatively, potentially the combination of these variables (fold-change cytokine levels and the risk score) is more informative (and support more accurate predictions) than either of the variables alone.

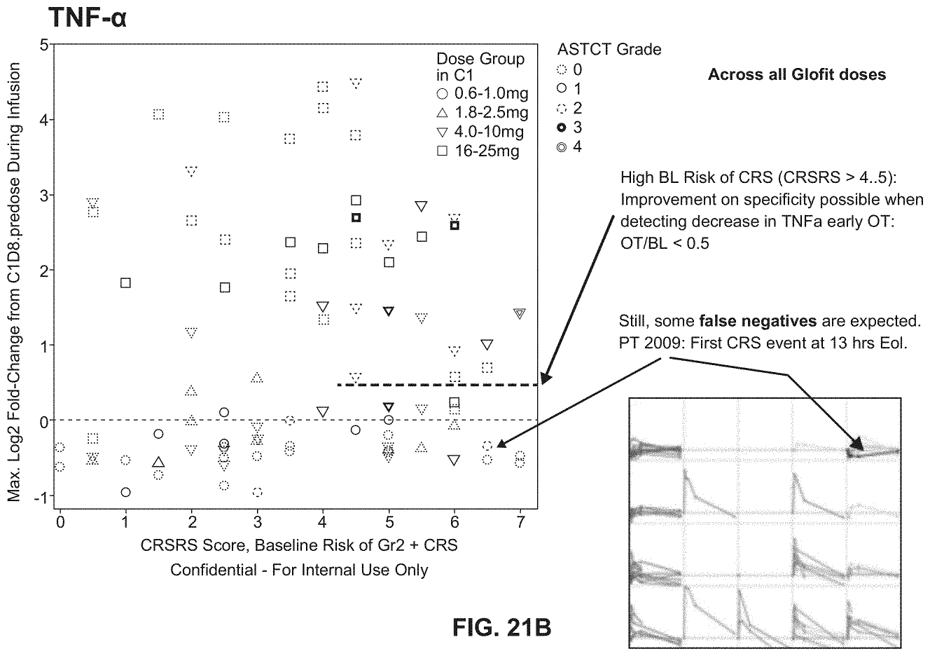

To investigate these issues, multi-dimensional plots were generated. Specifically, FIGS. 21A-21B show scatter plots that compare the maximum log 2 fold-changes of cytokine levels relatives to the cytokine release syndrome risk scores for various cytokines (across all dosages). The cytokine release syndrome risk score was calculated for each subject in accordance with the technique disclosed in Section IV.A.5.b.

A CRSRS threshold was defined to be 4.5, such that cytokine release syndrome risk scores above 4.5 were considered to represent a higher risk of a cytokine release syndrome of at least Grade 2 occurring as compared to lower risk scores. A fold-change threshold was also defined for each cytokine by first identifying the maximum cytokine fold change across all subjects associated with a cytokine release syndrome risk score less than 4.5 and by then averaging the values. The dashed line in each of FIGS. 21A-21B has a y-value equal to the fold-change threshold and extends across x range with a lower value equal to the CRSRS threshold.

For each cytokine, these thresholds were used to predict that a cytokine release syndrome of at least Grade 2 would occur when (1) a subject's cytokine release syndrome risk score was at least 4.5; and (2) when the subject's maximum log 2 fold-change of the cytokine exceeded the fold-change threshold. If either (or both) of these conditions was not satisfied, then it was predicted that the subject would not experience a cytokine release syndrome of at least Grade 2. Thus, in each of FIGS. 20A-20B, it was predicted that each data point that is above the dashed line corresponded to a cytokine release syndrome of at least Grade 2 and that each symbol that is below the dashed line or to the left of the dashed line did not correspond to a cytokine release syndrome of at least Grade 2.

Each red or purple symbol (corresponding to a Grade 2, 3, or 4 cytokine release syndrome) above the dashed line is a true positive. Each red or purple symbol below the dashed line or to the left of the dashed line is a false negative. Each green or blue symbol (corresponding to no cytokine release syndrome or a Grade 1 cytokine release syndrome) above the dashed line is a false positive. Each green or blue symbol below the dashed line or to the left of the dashed line is a true negative.

Across all of the five assessed cytokines, the vast majority of cytokine release syndromes of at least Grade 2 were associated with cytokine release syndrome risk scores and maximum log 2 fold-changes that exceeded the respective thresholds. However, some false negatives were observed. At least some of the false negatives may be due to the cytokine having a kinetic profile where a peak fold change was not reached during the infusion time period.

Using both criteria (pertaining to the CRSRS threshold and to the fold-change threshold) resulted in higher specificity values relative to using either individual threshold. Each specificity value was defined as the number of true negatives over the sum of true negatives and false positives.

### IV.B.6. Interpretations

Both the incidence and severity of cytokine release syndromes are strongly dose-dependent phenomena, as are the incidence and degree of cytokine induction. (See, e.g., FIGS. 16A, 16B, and 19A-19B.) Assessment of the predictive value of a cytokine signal to predict cytokine release syndrome incidence and severity is thus very challenging in Phase-I non-randomized studies without comparator arms and/or control for confounding factors.

With respect to each several cytokines (e.g., TNF-α, IL-8, M1P1b, IL-6 and IL-10), an association between on-therapy kinetics with the incidence and severity of cytokine release syndrome was observed.

When cytokine levels alone were evaluated to determine whether the levels were predictive of cytokine release syndrome incidence or Grade, the cytokine fold change delivered reasonable predictions. Kinetics of some cytokines (e.g., IL-6) may suggest that computing the cytokine fold changes using a post-infusion and baseline levels may be more predictive of the cytokine release syndromes as compared to cytokine fold changes computed using on-treatment and baseline levels for one or more cytokines. The magnitude of the cytokine fold changes were relatively small for some cytokines (1.4 to 2-fold increase for TNF-α and IL-8). When the differences between these subject groups are small, it may be advantageous or potentially even required to develop an assay with high sensitivity to predict cytokine release syndromes based on the cytokine level with sufficient reliability or accuracy.

Cytokine changes alone may be insufficient to achieve a reliably accuracy prediction of the incidence of severe cytokine release syndromes (of grade 2 or higher), both in terms of positive predictive values and negative predictive values. Improved predictive values can be achieved when combining early cytokine changes with baseline cytokine release syndrome risk scores.

### IV.C. Example 3: Exemplary Training and Use of Multi-Variate Model to Predict Occurrence of Cytokine Release Syndromes

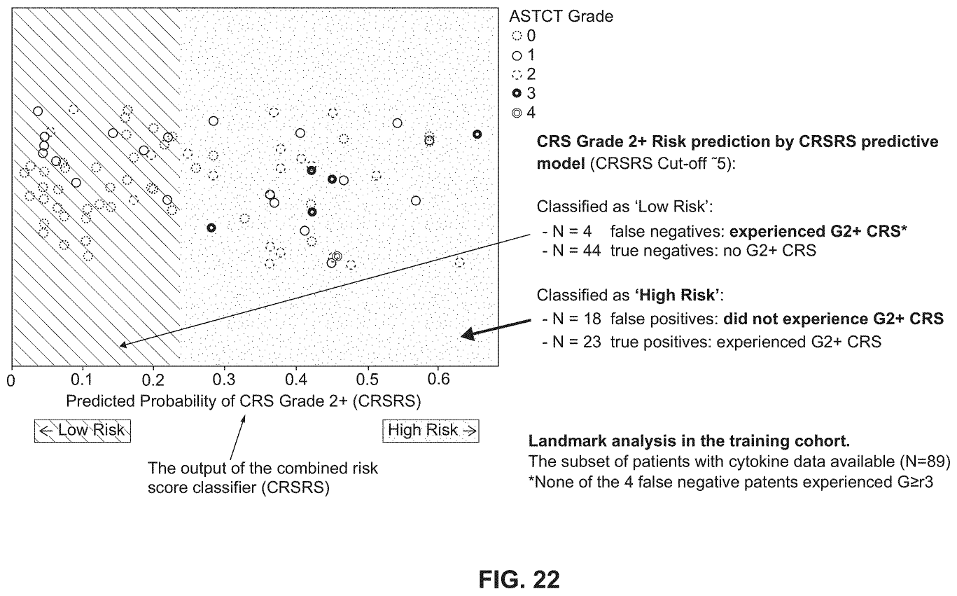

To determine an extent to which the cytokine release syndrome risk score could be used to reliably predict incidences of cytokine release syndromes (of grade 2 or higher), a score threshold for the score was determined. More specifically, a threshold was learned using training data to be one that best separates instances where a cytokine release syndrome of at least Grade 2 was observed versus instances where no such event was observed or a cytokine release syndrome of Grade 1 was observed. FIG. 22 shows results from a landmark analysis of how the probability of a grade 2 or higher cytokine release syndrome occurring (adjusted for the first glofitamab dosage) relates to a normalized version the cytokine release syndrome risk score. Only events occurring after the end of infusion are represented. Specifically, each data point represents a subject who had been diagnosed with aggressive non-Hodgkin's lymphoma and who received a treatment glofitamab. The color of the symbol represents a grade of cytokine release syndrome that was observed (if any, with dark green symbols representing that no cytokine release syndrome was observed). A jitter was introduced along the y-axis, meaning that the y-value of a symbol does not represent any characteristic of any cytokine release syndrome or of the subject.

Of the 89 subjects for which cytokine data was available, comparing the cytokine release syndrome risk score to the score threshold resulted in predicting that 41 of the subjects (46%) were of high risk of experiencing a cytokine release syndrome of grade 2 or higher and that 48 subjects (54%) were of low risk of experiencing a cytokine release syndrome of grade 2 or higher. Of the subjects predicted to be of high risk, 23 of those subjects did experience a cytokine release syndrome of grade 2 or higher, while 18 did not. Of the subjects predicted to be of low risk, 4 of those subjects did experience a cytokine release syndrome of grade 2 (though none of those 4 subjects experienced a cytokine release syndrome of grade 3 or higher), and 44 of those subjects did not.

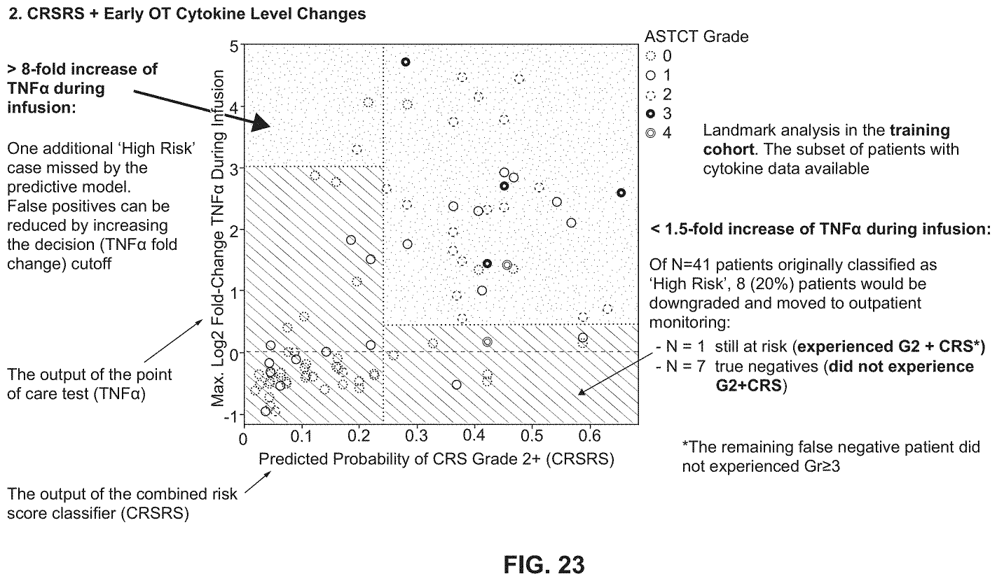

FIGS. 22 and 23 show how the grade of any observed cytokine release syndrome relates to both the cytokine release syndrome risk score and the cytokine fold change of TNF-α. Thus, each data point represented in FIG. 22 has a corresponding data point represented in FIG. 23, where the x-axis value is identical. However, the y-axis value in FIG. 23 indicates the cytokine fold change of TNF-α, FIG. 23 shows the same score threshold along the x-axis (corresponding to cytokine release syndrome risk scores) as shown in FIG. 22.

FIG. 23 further depicts two cytokine-change thresholds (learned using training data) along the y-axis, corresponding to thresholds for the cytokine fold change of TNF-α. Specifically, different cytokine-change thresholds for the cytokine fold change of TNF-α were identified, where a higher cytokine-change threshold for the cytokine fold change of TNF-α was selected for cytokine release syndrome risk scores were below the score threshold relative to the cytokine-change threshold selected for cytokine release syndrome risk scores were above the score threshold.

In this analysis, for each subject, a cytokine release syndrome risk score was used to identify a cutoff for TNF-α, where the threshold was selected so that a cytokine release syndrome risk score of 5 could be used to differentiate between subjects of low and high risk of experiencing a cytokine release syndrome of at least grade 2. As shown in FIG. 23, the vast majority the observed cytokine release syndromes of grade 2 or above were observed in subjects predicted to be high risk (24 out of 28). Further, the vast majority of instances for which a cytokine release syndrome of grade 2 or above was not observed corresponded to subjects predicted to be low risk (54 subjects) were accurately predicted (with only false negatives). Thus, the accuracy, precision, and recall of the predictions generated based on both the cytokine fold changes of TNF-α and also the cytokine release syndrome risk score were better than those based only on the cytokine release syndrome risk score.

### IV.D. Example 4: Exemplary Baseline-Characteristic Weights

In Example 1, Table 9 showed weights assigned to a set of baseline characteristics (or binary transformations thereof), and the weights were used to generate cytokine release syndrome risk scores. However, in some instances, values for all of variables corresponding to the baseline characteristics identified in Table 9 are not available. For example, tests to detect atypical cells in peripheral blood (e.g., a blood smear test) are not routinely performed. Further, at a time point when a treatment is to be determined, a bone marrow sample (to determine bone marrow infiltration) may be unavailable or the results of an infiltration analysis may be unavailable.

Therefore, the cytokine release syndrome risk score may be based on a reduced set of baseline characteristics. Table 12 identifies an exemplary reduced set of baseline characteristics. The weight for each characteristic in the reduced set of baseline characteristic was defined to be the same as the weight determined when the full set of baseline characteristics were analyzed.

TABLE 12

| Parameter & Cutoff | Weight |
|---|---|
| Lactate Dehydrogenase (LDH) > 280 U/l | 0.5 |
| White blood cell count (WBC) >4.5 * 10⁹ Cells/l | 0.5 |
| Age > 64 yrs | 1 |
| Ann Arbor Stage = III or IV | 2 |
| Sum of Product of Tumor Diameters (SPD) >= 3000 mm² | 2 |

When a reduced set of baseline characteristics is used, a confidence of a predicted output can be reduced. Thus, a confidence cut-off to discriminate between a predicted occurrence of cytokine release syndrome and a predicted non-occurrence of cytokine release syndrome was lowered to from 5 to 4 based on analysis of data from the validation cohort (2.5/10/30 mg SUD).

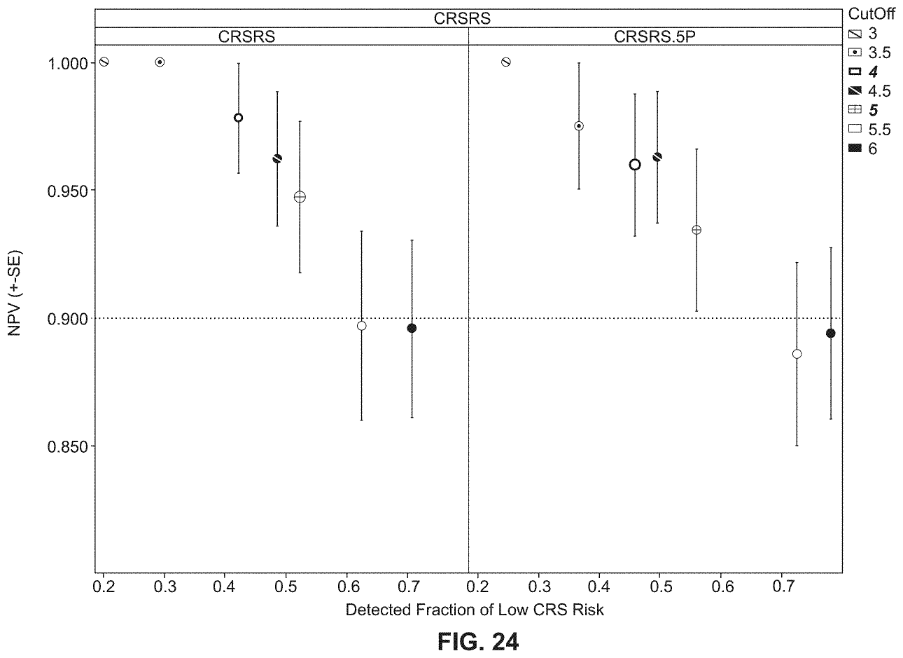

FIG. 24 shows the negative predictive value and low-risk detection rate for a set of cut-off values in the SUD cohort (n=109, aNHL cases). The left panel shows data corresponding to original cytokine release syndrome risk scores (CRSRS calculated using the 8 baseline characteristics as identified in Table 9), and the right panel shows data corresponding to the reduced set of baseline characteristics (CRSRS.5p as identified in Table 12).

Table 13 shows exemplary performance metrics for predictions using each of two adjusted confidence cut-off (4 or 5) and for each of the two set of baseline characteristics. Specifically, Table 13 shows performance metrics when the predictions were made using the baseline characteristics identified in Table 9, and Table shows performance metrics when predictions were made using baseline characteristics identified in Table 12. Further, the first row in each table corresponds to a cut-off of 4.0 (for converting real-value outputs to binary predictions), and the second row in each table corresponds to a cut-off of 5.0. Missing values were imputed with zeros, thereby corresponding to a ‘base case’ scenario and may underestimate the baseline risk. Using the adjusted confidence cut-off of 4, the predictive performance of the reduced classifier was comparable to that of the performance of the classifier that used 8 baseline characteristics.

TABLE 13

| CRSRS | Predicted Negative | | | Predicted Positive | | | |
|---|---|---|---|---|---|---|---|
| CutOff | Cases, N (%) | NPV (SE) | N at risk | Cases, N (%) | PPV (SE) | Sensitivity | Specificity |
| 4.0 | 46 (42%) | 0.98 (0.02) | 1° | 63 (58%) | 0.27 (0.06) | 0.94 | 0.50 |
| 5.0 | 57 (52%) | 0.95 (0.03) | 3°° | 52 (48%) | 0.30 (0.06) | 0.83 | 0.59 |

°1 DLBCL;
°°1 DLBCL, 1 trFL, 1 FL3B

TABLE 14

| CRSRS | Predicted Negative | | | Predicted Positive | | | |
|---|---|---|---|---|---|---|---|
| CutOff | Cases, N (%) | NPV (SE) | N at risk | Cases, N (%) | PPV (SE) | Sensitivity | Specificity |
| 4.0 | 50 (46%) | 0.96 (0.02) | 2° | 59 (54%) | 0.27 (0.06) | 0.90 | 0.53 |
| 5.0 | 61 (44%) | 0.93 (0.03) | 4°° | 48 (44%) | 0.29 (0.06) | 0.80 | 0.63 |

°1 DLBCL, 1 FL3B;
°°1 DLBCL, 1 FL3B, 2 trFL

### IV. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification, and variation of the concepts herein disclosed may be resorted to by those skilled in the art, and that such modifications and variations are considered to be within the scope of this invention as defined by the appended claims.

The description provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1. A computer-implemented method comprising:
identifying a set of baseline characteristics of a subject who has been diagnosed with cancer, wherein the set of baseline characteristics pertain to one or more baseline time points that are before initiation of a treatment, and wherein each of the set of baseline characteristics characterize:
a stage of the cancer;
a demographic attribute;
a size of one or more tumors;
a white blood cell count; and/or
a lactate dehydrogenase level;
generating a numeric cytokine release syndrome risk score by processing the set of baseline characteristics using a risk-score generation model;
predicting, based on the numeric cytokine release syndrome risk score, a risk of the subject experiencing a cytokine release syndrome of at least a threshold grade subsequent to receiving the treatment;
determining a result based on the predicted risk corresponding to a recommendation as to whether to monitor the subject via in-patient monitoring subsequent to completion of the treatment; and
outputting the result.

2. The computer-implemented method of claim 1, wherein at least one of the set of baseline characteristics characterizes a stage of the cancer.

3. The computer-implemented method of claim 1, wherein at least one of the set of baseline characteristics characterizes a lactate dehydrogenase level.

4. The computer-implemented method of claim 1, wherein at least one of the set of baseline characteristics characterizes a white blood cell count.

5. The computer-implemented method of claim 1, wherein at least one of the set of baseline characteristics characterizes a size of the one or more tumors.

6. The computer-implemented method of claim 1, wherein at least one of the set of baseline characteristics characterizes a demographic attribute.

7. The computer-implemented method of claim 1, further comprising:
determining the result based on the predicted risk, wherein the result corresponds to a recommendation as to whether to monitor the subject via in-patient monitoring subsequent to completion of the treatment.

8. The computer-implemented method of claim 1, further comprising:
identifying an on-treatment level of a cytokine, wherein the on-treatment level of the cytokine indicates a level of the cytokine in an on-treatment sample collected from the subject while the treatment was being administered or within an hour of completion of the treatment;
determining an on-treatment cytokine fold change of the cytokine based on the on-treatment level of the cytokine and a baseline level of the cytokine that indicates a level of the cytokine in a baseline sample collected from the subject before initiation of the treatment;
wherein the predicted risk is further based on the on-treatment cytokine fold change.

9. The computer-implemented method of claim 1, further comprising:
identifying a dosage of at least prat of the treatment, wherein the predicted risk is further based on the dosage.

10. The computer-implemented method of claim 1, wherein the risk-score generation includes a regression model.

11. The computer-implemented method of claim 1, wherein the treatment includes administering a T cell immunotherapy.

12. The computer-implemented method of claim 1, wherein the treatment includes administering glofitamab or mosunetuzumab.

13. The computer-implemented method of claim 1, wherein the treatment comprises administering a therapy that comprises an antibody or a small molecule.

14. The computer-implemented method of claim 13, wherein the administered therapy comprises an antibody.

15. The computer-implemented method of claim 14, wherein the antibody is a multispecific antibody that engages T-cells when bound to at least one of its antigens.

16. A computer-implemented method comprising:
identifying an on-treatment level of a cytokine, wherein the on-treatment level of the cytokine indicates a level of the cytokine in an on-treatment sample collected from a subject while a treatment was being administered or within an hour of completion of the treatment;
determining an on-treatment cytokine fold change of the cytokine based on the on-treatment level of the cytokine and a baseline level of the cytokine that indicates a level of the cytokine in a baseline sample collected from the subject before initiation of the treatment;
identifying a dosage of at least part of the treatment;
predicting, based on the on-treatment cytokine fold change and the dosage, a risk of the subject experiencing a cytokine release syndrome of at least a threshold grade subsequent to receiving the dosage of the at least part of the treatment;
determining a result based on the predicted risk corresponding to a recommendation as to whether to monitor the subject via in-patient monitoring subsequent to completion of the treatment; and
outputting the result.

17. The computer-implemented method of claim 16, further comprising:
identifying a set of baseline characteristics of the subject, wherein the set of baseline characteristics pertain to one or more baseline time points that are before the initiation of the treatment, and wherein each of the set of baseline characteristics characterize:
a tumor burden;
a stage of cancer;
a tumor spread;
a size of one or more tumors;
a demographic attribute;
a white blood cell count; and/or
a lactate dehydrogenase level;
wherein the predicted risk further depends on the set of baseline characteristics.

18. The computer-implemented method of claim 17, further comprising:
generating a cytokine release syndrome risk score by processing the set of baseline characteristics with a risk-score generation model, wherein the predicted risk is based on the cytokine release syndrome risk score.

19. The computer-implemented method of claim 18, wherein the risk-score generation includes a regression model.

20. The computer-implemented method of claim 18, wherein the one or more parameters include a set of weights.

21. The computer-implemented method of claim 18, wherein the risk is determined based on a linear combination of the cytokine release syndrome risk score and the dosage.

22. The computer-implemented method of claim 16, wherein predicting the risk that the subject will experience the cytokine release syndrome includes performing one or more threshold comparisons.

23. The computer-implemented method of claim 16, wherein the subject has been diagnosed with cancer, and wherein the treatment includes administering a T cell immunotherapy.

24. The computer-implemented method of claim 16, wherein the subject has been diagnosed with cancer, and wherein the treatment includes administering glofitamab or mosunetuzumab.

25. The computer-implemented method of claim 16, wherein determining the on-treatment cytokine fold change of the cytokine based on the baseline level of the cytokine includes:
calculating a log of the baseline level of the cytokine or of a processed version thereof to generate a baseline log value;
calculating a log of the on-treatment level of the cytokine or a processed version thereof to generate an on-treatment log value; and
subtracting the baseline log value from the on-treatment log value.

26. The computer-implemented method of claim 16, wherein determining the on-treatment cytokine fold change of the cytokine based on the baseline level of the cytokine includes:
calculating a log of a difference between the baseline level of the cytokine and a constant to generate a baseline log value;
calculating a log of a difference between the on-treatment level of the cytokine and the constant to generate an on-treatment log value; and
subtracting the baseline log value from the on-treatment log value.

27. The computer-implemented method of claim 16, wherein identifying the on-treatment level of the cytokine includes:
identifying multiple preliminary on-treatment levels of the cytokine that indicate levels of the cytokine in multiple on-treatment samples collected from the subject while the treatment was being administered or within a day of completion of the treatment, wherein each of the multiple on-treatment samples was collected at a different time; and
defining the on-treatment level of the cytokine to be a maximum of the multiple preliminary on-treatment levels of the cytokine.

28. The computer-implemented method of claim 16, wherein:
the treatment includes administering an active ingredient; and
the treatment was preceded by administering a pre-treatment with another agent.

29. The computer-implemented method of claim 28, wherein the on-treatment level was identified using a sample collected after administration of the active ingredient.

30. The computer-implemented method of claim 16, wherein the cytokine includes Tumor Necrosis Factor alpha, Interleukin 6, Interleukin 8, Interleukin 10, or Macrophage Inflammatory Protein 1 beta.

31. The computer-implemented method of claim 29, wherein the treatment comprises administering a therapy that comprises an antibody or a small molecule.

32. A system comprising:
one or more data processors; and
a non-transitory computer readable storage medium containing instructions which, when executed on the one or more data processors, cause the one or more data processors to perform the method of any of claims 1-31.

33. A computer-program product tangibly embodied in a non-transitory machine-readable storage medium, including instructions configured to cause one or more data processors to perform the method any of claims 1-31.


SEQUENCE LISTING (50 sequences)

SEQ ID NO: 1 - protein, 10 aa, synthetic construct
GYTFTSYNMH

SEQ ID NO: 2 - protein, 17 aa, synthetic construct
AIYPGNGDTSYNQKFKG

SEQ ID NO: 3 - protein, 13 aa, synthetic construct
VVYYSNSYWYFDV

SEQ ID NO: 4 - protein, 10 aa, synthetic construct
RASSSVSYMH

SEQ ID NO: 5 - protein, 7 aa, synthetic construct
APSNLAS

SEQ ID NO: 6 - protein, 9 aa, synthetic construct
QQWSFNPPT

SEQ ID NO: 7 - protein, 122 aa, synthetic construct [sequence omitted]

SEQ ID NO: 8 - protein, 106 aa, synthetic construct [sequence omitted]

SEQ ID NO: 9 - protein, 5 aa, synthetic construct
NYYIH

SEQ ID NO: 10 - protein, 17 aa, synthetic construct
WIYPGDGNTKYNEKFKG

SEQ ID NO: 11 - protein, 10 aa, synthetic construct
DSYSNYYFDY

SEQ ID NO: 12 - protein, 17 aa, synthetic construct
KSSQSLLNSRTRKNYLA

SEQ ID NO: 13 - protein, 7 aa, synthetic construct
WASTRES

SEQ ID NO: 14 - protein, 8 aa, synthetic construct
TQSFILRT

SEQ ID NO: 15 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 17 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 672 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 22 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 23 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 674 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 449 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 232 aa, synthetic construct; identical to SEQ ID NO: 24

SEQ ID NO: 28 - protein, 219 aa, synthetic construct; identical to SEQ ID NO: 23

SEQ ID NO: 29 - protein, 127 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSAS

SEQ ID NO: 30 - protein, 111 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSS

SEQ ID NO: 31 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 32 - protein, 119 aa, synthetic construct; identical to SEQ ID NO: 31

SEQ ID NO: 33 - protein, 5 aa, synthetic construct
YSWIN

SEQ ID NO: 34 - protein, 17 aa, synthetic construct
RIFPGDGDTDYNGKFKG

SEQ ID NO: 35 - protein, 10 aa, synthetic construct
NVFDGYWLVY

SEQ ID NO: 36 - protein, 16 aa, synthetic construct
RSSKSLLHSNGITYLY

SEQ ID NO: 37 - protein, 7 aa, synthetic construct
QMSNLVS

SEQ ID NO: 38 - protein, 9 aa, synthetic construct
AQNLELPYT

SEQ ID NO: 39 - protein, 5 aa, synthetic construct
TYAMN

SEQ ID NO: 40 - protein, 19 aa, synthetic construct
RIRSKYNNYATYYADSVKG

SEQ ID NO: 41 - protein, 14 aa, synthetic construct
HGNFGNSYVSWFAY

SEQ ID NO: 42 - protein, 14 aa, synthetic construct
GSSTGAVTTSNYAN

SEQ ID NO: 43 - protein, 7 aa, synthetic construct
GTNKRAP

SEQ ID NO: 44 - protein, 9 aa, synthetic construct
ALWYSNLWV

SEQ ID NO: 45 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 46 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 47 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 48 - protein, 297 aa, Homo sapiens; identical to SEQ ID NO: 47

SEQ ID NO: 49 - DNA, 3331 bp, Homo sapiens
  source 1..3331: mol_type = genomic DNA
gtctatcagcgatttcatcttcaggcctggactacaccactcaccctcccagtgtgcttgagaaacaaactgcacccactgaactccgcagctagcatccaaatcagcccttgagatttgaggccttggagactcaggagttttgagagcaaaatgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaagtgatttcttctgttttctgtttccttttttaaacattagtgttcatagcttccaagagacatgctgactttcatttcttgaggtactctgcacatacgcaccacatctctatctggcctttgcatggagtgaccatagctccttctctcttacattgaatgtagagaatgtagccattgtagcagcttgtgttgtcacgcttcttcttttgagcaactttcttacactgaagaaaggcagaatgagtgcttcagaatgtgatttcctactaacctgttccttggataggctttttagtatagtatttttttttgtcattttctccatcaacaaccagggagactgcacctgatggaaaagatatatgactgcttcatgacattcctaaactatcttttttttattccacatctacgtttttggtggagtcccttttgcatcattgttttaaggatgataaaaaaaaataacaactagggacaatacagaacccattccatttatctttctacagggctgacattgtggcacattcttagagttaccacaccccatgagggaagctctaaatagccaacacccatctgttttttgtaaaaacagcatagcttatacatggacatgtctctgccttaacttttcctaactcccactctaggctattgtttgcatgtctacctacttttagccattatgcgagaaaagaaaaaaatgaccatagaaaatgccaccatgaggtgcccaaatttcaaataataattaacatttagttatatttataatttccagatgacaaagtatttcatcaaataacttcatttgatgttccatgatcaagaaagaatccctatctctattttacaagtaattcaaagaggccaaataacttgtaaacaagaaaaggtaacttgtcaacagtcataactagtaattatgagagccttgtttcataaccaggtcttcttactcaaatcctgtgatgtttgaaataaccaaattgtctctccaatgtctgcataaactgtgagagccaagtcaacagcttttatcaagaatttactctctgaccagcaataaacaagcactgagagacacagagagccagattcagattttacccatggggataaaaagactcagactttcaccacatttggaaaactacttgcatcataaatatataataactggtagtttatatgaagcagacactaagtgctatagacactctcagaatatcatacttggaaacaatgtaattaaaatgccgaatctgagtcaacagctgccctacttttcaattcagatatactagtaccttacctagaaataatgttaacctagggtgaagtcactataatctgtagtctattatttgggcatttgctacatgatgagtgctgccagattgtggcaggtaaagagacaatgtaatttgcactccctatgatatttctacatttttagcgaccactagtggaagacattccccaaaattagaaaaaaaggagatagaagatttctgtctatgtaaagttctcaaaatttgttctaaattaataaaactatctttgtgttcttttctgcaacagatgattccaacatgggtgtttgtctattcttctttactcttgaaacattagaccatgggaggctcttacagccttgagttgatatttatacaacccaaatctaggtttgaacggtgaggtgtcaggtcatcaaatattcatgtctatatagtcttacacaggttctcaaaaaaaatgttcatgggataggtcattgataatggattccttattctgagaactccagacgactgaaatatatgagagaaggaaaaggacatagtaggagcaggcctgagaaaaaaatgaaagtcagaaatctttaaaaaaatacaagatcttatttctatcttattttttctcctcttctgaaatatatatgaggattcctctccaaacccatggtttctctaagaattttgagtcatttgtatgacctcaaataattagttttagctgacctcacataactccttataataggagacatctttaatgtctgctattaaagaaggatgaaaattcctatgaccttctccccgattatccctttggcaatatagagtcaaataataacattgaccaatagtaaacatgctttgccaagaagtagaagatatattctctagccttagtttttcctcccaatttgcatttttgtaaaaataatgttgtatccacaaaggaaataaactttaaaaacccaagtgca

SEQ ID NO: 50 - DNA, 3594 bp, Homo sapiens
  source 1..3594: mol_type = genomic DNA
gtctatcagcgatttcatcttcaggcctggactacaccactcaccctcccagtgtgcttgagaaacaaactgcacccactgaactccgcagctagcatccaaatcagcccttgagatttgaggccttggagactcagatcctgaacaagagagaacaaaatctctactttgatggaacttccattctgtggggaagagactgacaataagcaattaaataaataagaactcagcagtaggccttgcctcagatccaaggtcactcggaagaggccatgtctaccctcaatgacactcatggaggaaatgctgagagaagcattcagatgcatgacacaaggtaagactgccaaaaatcttgttcttgctctcctcattttgttatttgttttatttttaggagttttgagagcaaaatgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaagtgatttcttctgttttctgtttccttttttaaacattagtgttcatagcttccaagagacatgctgactttcatttcttgaggtactctgcacatacgcaccacatctctatctggcctttgcatggagtgaccatagctccttctctcttacattgaatgtagagaatgtagccattgtagcagcttgtgttgtcacgcttcttcttttgagcaactttcttacactgaagaaaggcagaatgagtgcttcagaatgtgatttcctactaacctgttccttggataggctttttagtatagtatttttttttgtcattttctccatcaacaaccagggagactgcacctgatggaaaagatatatgactgcttcatgacattcctaaactatcttttttttattccacatctacgtttttggtggagtcccttttgcatcattgttttaaggatgataaaaaaaaataacaactagggacaatacagaacccattccatttatctttctacagggctgacattgtggcacattcttagagttaccacaccccatgagggaagctctaaatagccaacacccatctgttttttgtaaaaacagcatagcttatacatggacatgtctctgccttaacttttcctaactcccactctaggctattgtttgcatgtctacctacttttagccattatgcgagaaaagaaaaaaatgaccatagaaaatgccaccatgaggtgcccaaatttcaaataataattaacatttagttatatttataatttccagatgacaaagtatttcatcaaataacttcatttgatgttccatgatcaagaaagaatccctatctctattttacaagtaattcaaagaggccaaataacttgtaaacaagaaaaggtaacttgtcaacagtcataactagtaattatgagagccttgtttcataaccaggtcttcttactcaaatcctgtgatgtttgaaataaccaaattgtctctccaatgtctgcataaactgtgagagccaagtcaacagcttttatcaagaatttactctctgaccagcaataaacaagcactgagagacacagagagccagattcagattttacccatggggataaaaagactcagactttcaccacatttggaaaactacttgcatcataaatatataataactggtagtttatatgaagcagacactaagtgctatagacactctcagaatatcatacttggaaacaatgtaattaaaatgccgaatctgagtcaacagctgccctacttttcaattcagatatactagtaccttacctagaaataatgttaacctagggtgaagtcactataatctgtagtctattatttgggcatttgctacatgatgagtgctgccagattgtggcaggtaaagagacaatgtaatttgcactccctatgatatttctacatttttagcgaccactagtggaagacattccccaaaattagaaaaaaaggagatagaagatttctgtctatgtaaagttctcaaaatttgttctaaattaataaaactatctttgtgttcttttctgcaacagatgattccaacatgggtgtttgtctattcttctttactcttgaaacattagaccatgggaggctcttacagccttgagttgatatttatacaacccaaatctaggtttgaacggtgaggtgtcaggtcatcaaatattcatgtctatatagtcttacacaggttctcaaaaaaaatgttcatgggataggtcattgataatggattccttattctgagaactccagacgactgaaatatatgagagaaggaaaaggacatagtaggagcaggcctgagaaaaaaatgaaagtcagaaatctttaaaaaaatacaagatcttatttctatcttattttttctcctcttctgaaatatatatgaggattcctctccaaacccatggtttctctaagaattttgagtcatttgtatgacctcaaataattagttttagctgacctcacataactccttataataggagacatctttaatgtctgctattaaagaaggatgaaaattcctatgaccttctccccgattatccctttggcaatatagagtcaaataataacattgaccaatagtaaacatgctttgccaagaagtagaagatatattctctagccttagtttttcctcccaatttgcatttttgtaaaaataatgttgtatccacaaaggaaataaactttaaaaacccaagtgca